(19) United States
(12) Patent Application Publication
(10) Pub. No.: US 2022/0242812 A1
(43) Pub. Date: Aug. 4, 2022

(54) SWELL1-LRRC8 COMPLEX MODULATORS

(71) Applicants: Washington University, St. Louis, MO (US); University of Iowa Research Foundation, Iowa City, MO (US)

(72) Inventors: Rajan Sah, St. Louis, MO (US); Robert Kerns, Iowa City, IA (US); Pratik Chheda, Iowa City, IA (US)

(73) Assignees: Washington University, St. Louis, MO (US); University of Iowa, Iowa City, IA (US)

(21) Appl. No.: 17/617,850

(22) PCT Filed: Jun. 10, 2020
(86) PCT No.: PCT/US2020/037022

§ 371 Date: Dec. 9, 2021

Related U.S. Application Data
(60) Provisional application No. 62/982,531, filed on Feb. 27, 2020.
(60) Provisional application No. 62/963,988, filed on Jan. 21, 2020.
(60) Provisional application No. 62/859,499, filed on Jun. 10, 2019.

(51) Int. Cl. C07C 61/40; C07C 59/90; C07C 233/08; C07C 217/22; C07D 257/04; A61P 3/00

(52) U.S. Cl. CPC C07C 61/40; C07C 59/90; A61P 3/00; C07C 217/22; C07D 257/04; C07C 233/08

(57) ABSTRACT

The present invention is directed to various polycyclic compounds and methods of using these compounds to treat a variety of diseases including metabolic diseases such as obesity, diabetes, nonalcoholic fatty liver disease; cardiovascular diseases such as hypertension and stroke; neurological diseases, male infertility, muscular disorders, and immune disorders.

### FIELD OF THE INVENTION

The present invention is directed to various polycyclic compounds and methods of using these compounds to treat a variety of diseases associated with abnormal SWELL1 signaling including metabolic diseases such as obesity, diabetes, nonalcoholic fatty liver disease; cardiovascular diseases such as hypertension and stroke; neurological diseases; male infertility, muscular disorders, and immune deficiencies.

### BACKGROUND OF THE INVENTION

Obesity-induced diabetes (Type 2 diabetes, T2D) is reaching epidemic proportions with more than one in three Americans obese (36%), >29 million with diabetes and ˜86 million with pre-diabetes in the US alone (in 2014, CDC). The economic consequences of obesity and diabetes in the US alone are close to $500 billion. Globally, this is an even more significant problem, where the incidence of Type 2 diabetes is estimated at 422 million in 2014 and the projected numbers are expected to reach over 700 million within the next decade. Non-alcoholic fatty liver disease (NAFLD), is highly associated with T2D, and has a prevalence of 24% in both the US and globally. NAFLD often progresses to advanced liver disease, cirrhosis and hepatocellular carcinoma, and is currently the second most common indication for liver transplantation in the US, after hepatitis C.

While there are currently several commercially available drugs to treat Type 2 diabetes, physicians remain challenged with effectively treating this disease, as a significant percentage of patients continue to have poorly controlled blood glucose, despite optimal medical therapy. Failure of medical therapy relates to a number of factors, including a narrow mechanism of action (insulin sensitizer vs. secretagogue vs. other), medication non-compliance (particularly for drugs with frequent dosing regimens) and achieving euglycemia while avoiding life-threatening hypoglycemia. Moreover, several current therapies suffer from unwanted and dangerous side effects such as congestive heart failure, weight gain and edema including TZDs that are also used for NAFLD.

Volume regulated anion channels (VRAC) are considered cell swelling-induced anion channels. They modulate vital functions in a variety of organ systems and have been implicated in pathology associated with diabetes, obesity, non-alcoholic fatty liver disease, stroke, hypertension and other conditions. The leucine-rich repeat-containing protein 8A (LRRC8A) which is also known as SWELL1, along with its four other associated homologs (LRRC8B-E) form heteromeric VRACs.

SWELL1 (LRRC8a) is a required component of a volume-sensitive ion channel molecular complex that is activated in the setting of adipocyte hypertrophy and regulates adipocyte size, insulin signaling and systemic glycaemia via a novel SWELL1-PI3K-AKT2-GLUT4 signaling axis. Adipocyte-specific SWELL1 ablation disrupts insulin-PI3K-AKT2 signaling, inducing insulin resistance and glucose intolerance in vivo. As such, SWELL1 is identified as a positive regulator of adipocyte insulin signaling and glucose homeostasis, particularly in the setting of obesity.

In addition to impaired insulin sensitivity, Type 2 diabetes is also characterized by a relative loss of insulin-secretion from the pancreatic β-cell. Regulation of β-cell excitability is a dominant mechanism controlling insulin secretion and systemic glycaemia. Indeed, a cornerstone of current diabetes pharmacotherapy, the sulfonylurea receptor inhibitors (i.e., glibenclamide), are aimed at antagonizing the well-characterized, inhibitory, hyperpolarizing current I_{K,ATP} to facilitate β-cell depolarization, activate voltage-gated calcium channels (VGCC) and thereby trigger insulin secretion. However, in order for such agents to be effective, an excitatory current must exist to allow for membrane depolarization. SWELL1 is required for a prominent swell-activated chloride current in β-cells. SWELL1-mediated VRAC is activated by glucose-mediated β-cell swelling, providing an essential depolarizing current required for β-cell depolarization, glucose-stimulated Ca2+ signaling and insulin secretion.

Normal SWELL1 function is required for normal human immune system development. In one example, expression of a truncated SWELL1 protein caused by a translocation in one allele of SWELL1 inhibits normal β-cell development, causing agammaglobulinemia 5 (AGMS) (Sawada, A., et al. Journal of Clinical Investigation 2003; Kubota, K. et al., FEBS Lett 2004). Because different types of immune system cells (e.g., B-lymphocytes and T-lymphocytes) use similar intracellular signaling pathways, it is likely that the development and/or function of other immune system cells (e.g., T-lymphocytes, macrophages, and/or NK cells) would also be affected in adequate SWELL1 function.

Currently, the molecular causes of male infertility are only partially understood. In mice lacking SWELL1 late spermatids fail to reduce their cytoplasm during development into spermatozoa and have disorganized mitochondrial sheaths with angulated flagella, resulting in reduced sperm motility. This demonstrates that SWELL1 is also required for normal spermatid development and male fertility (Luck, J. C., Journal of Biological Chemistry 2018).

SWELL1 and associated VRAC signaling is also linked to stroke induced neurotoxicity and cardiovascular disease.

There is evidence that a variety of conditions may be treated by inhibiting or otherwise modulating SWELL1 using compounds that directly bind to it. One such compound is DCPIB (4-[2[butyl-6,7-dichloro-2-cyclopentyl-2,3-dihydro-1-oxo-1H-inden-5-yl)oxy]butanoic acid) (herein referred to as Smod1) described in WO2018/027175, which has affinity for LRRC8A. However, there exists a need for compounds that have improved affinity and metabolic profiles and that target a larger variety of LRRC8 homologs. Such compounds can be useful for improved therapies for diabetes, obesity, non-alcoholic fatty liver disease, stroke, hypertension, immune deficiencies, male infertility, and other conditions.

### BRIEF SUMMARY

Various aspects of the present invention are directed to compounds of Formula (I), and salts thereof:

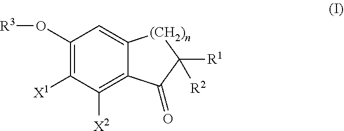

wherein:

R¹ and R² are each independently hydrogen, substituted or unsubstituted alkyl, substituted or unsubstituted alkenyl, substituted or unsubstituted alkoxy, substituted or unsubstituted aryl, substituted or unsubstituted heteroaryl;

R³ is —Y—C(O)R⁴, —Z—N(R⁵)(R⁶), or —Z-A;

R⁴ is hydrogen, substituted or unsubstituted alkyl, —OR⁷, or —N(R⁸)(R⁹);

X¹ and X² are each independently substituted or unsubstituted alkyl, halo, —OR¹⁰, or —N(R¹¹)(R¹²);

R⁵, R⁶, R⁷, R⁸, R⁹, R¹⁰, R¹¹ and R¹² are each independently hydrogen or substituted or unsubstituted alkyl;

Y and Z are each independently a substituted or unsubstituted carbon-containing moiety having at least 2 carbon atoms;

A is a substituted or unsubstituted 5- or 6-membered heterocyclic ring having at least one nitrogen heteroatom, boronic acid or

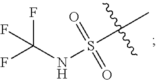 [SMILES: *S(=O)(=O)NC(F)(F)F]

and

n is 1 or 2.

Further aspects are directed to various methods using the compound of Formula (I) to treat various conditions in a subject in need thereof including insulin sensitivity, obesity, diabetes, nonalcoholic fatty liver disease, metabolic diseases, hypertension, stroke, vascular tone, systemic arterial and/or pulmonary arterial blood pressure, blood flow, male infertility, muscular disorders, and/or immune deficiencies. In general, the method comprises administering to the subject a therapeutically effective amount of a compound of Formula (I).

Other objects and features will be in part apparent and in part pointed out hereinafter.

### BRIEF DESCRIPTION OF THE DRAWINGS

FIG. 1. Chemical structures of Smod1/DCPIB, Smod4, Smod2, Smod3, Smod5, Smod6 and Snot1 as described herein.

FIG. 2. Patch-clamp screening of Smod compounds for I_{CL,SWELL} inhibitory activity. Outward (black) and inward (blue) current over time of I_{CL,SWELL} upon application of (A) Snot1: a Smod compound lacking I_{Cl,SWELL} inhibitory activity, (B) Smod2 maintaining activity, and (C) Smod3 maintaining activity.

FIG. 3. Patch-clamp screening of Smod compounds for I_{CL,SWELL} inhibitory activity. Outward (black) and inward (blue) current over time of I_{CL,SWELL} upon application of (A) Snot1: a Smod compound lacking I_{Cl,SWELL} inhibitory activity, (B) Smod3 maintaining and augmenting activity, (C) Smod4 maintaining activity, (D) Smod5 maintaining activity.

FIG. 4. Dose response curves plotting proportion of current (% control) with increasing concentrations of Smod3, Smod1 (+) and Smod1 (−). EC₅₀ of Smod(+) indicated with dashed red line and EC₅₀ of Smod3 indicated with dashed blue line.

FIG. 5. Synthesis of Smod1 and representative notations for alterations that will accommodate synthesis of Smod compounds. Modifications to the synthetic scheme that can be made to synthesize a variety of compounds described herein are indicated by double arrows. Methods: i) AlCl₃, DCM, 5° C. to rt. ii) 12N HCl. iii) 1) Paraformaldehyde, dimethylamine, acetic acid, 85° C. iv) DMF, 85° C., v) H₂SO₄. vi) KOtBu, butyl iodide. vii) pyridine-HCl, 195° C. viii) BrCH₂CO₂Et, K₂CO₃, DMF, 60° C. ix) 10N NaOH.

FIG. 6. SWELL1 protein induction in 3T3-F442A adipocytes by Smod3, and Smod5 but not vehicle or Snot1.

FIG. 7. Representative glucose tolerance test data, area under curve (AUC) and fasting glucose for mice treated with a vehicle, and 5 mg/kg/day Smod3 or Snot1 for 5 days. Smod3, but not Snot1 improves glucose tolerance (as measured by are under the curve, AUC), and fasting glucose in HFD T2D mice. N=5 mice in each group. *p<0.05, ** p<0.01, *** p<0.001.

FIG. 8. Glucose Tolerance of obese T2D mice (16 weeks HFD): Pre-Smod6 (black circles), after Smod6 (5 mg/kg i.p.×5 days, pink triangles), 4 weeks after i.p. vehicle injection (blue diamonds), and 4 weeks after discontinuing Smod6 (maroon squares).

FIG. 9. Glucose Tolerance of obese T2D mice (16 weeks HFD): 4 weeks after i.p. vehicle injection (black circles), 4 weeks after Snot1 (5 mg/kg i.p.×5 days, blue squares), and 4 weeks after Smod6 (5 mg/kg i.p.×5 days, maroon triangles).

FIG. 10. Cryo-electron microscopy structure of SWELL1 homo-hexamer with Smod1/DCPIB in the pore. The negatively charged carboxylate interacts electrostatically with a positively charged arginine (R103) from SWELL1/LRRC8a and/or LRRC8b at pore constriction. Figure adapted from Kern et al. eLife (2019).

FIG. 11. Docking of Smod1 into SWELL1 using structure PDB ID:6NZW. (A). Docking using Molecular Operating Environment (MOE) generated docking poses consistent with orientation of Smod1 observed in the Cryo-EM structure (FIG. 8). (B). Docking using SeeSAR with the LeadlT software package generated binding poses that scored higher than poses from the Cryo-EM structure, where Smod1 is flipped 180 degrees. (C) Overlay of highest scoring MOE (red) and SeeSAR (yellow) docked poses of Smod1 with SWELL1.

FIG. 12. Patch-clamp screening of UIPC-03-099 compound for I_{CL,SWELL} inhibitory activity at 10 μM.

FIG. 13. Patch-clamp screening of UIPC-03-099 compound for I_{CL,SWELL} inhibitory activity at 5 μM.

FIG. 14. Patch-clamp screening of UIPC-03-099 compound for I_{CL,SWELL} inhibitory activity at 5 μM.

FIG. 15. Patch-clamp screening of UIPC-03-099 compound for I_{CL,SWELL} inhibitory activity at 5 μM.

FIG. 16. Patch-clamp screening of UIPC-03-099 compound for I_{CL,SWELL} inhibitory activity at 1 μM.

FIG. 17 shows a reaction scheme for generating compounds SN-401, SN-403, SN-406, SN-407 and SN071.

FIG. 18 shows a reaction scheme for generating SN072.

FIG. 19 shows a reaction scheme for generating racemic compounds for SN-401.

FIG. 20A shows a current-voltage plot of I_{Cl,SWELL} measured in non-T2D and T2D mouse at baseline (iso, black trace) and; with hypotonic (210 mOsm) stimulation (hypo, grey trace).

FIG. 20B shows a current-voltage plots of I_{Cl,SWELL} measured in non-T2D and T2D human cells at baseline (iso, black trace) and; with hypotonic (210 mOsm) stimulation (hypo, grey trace).

FIG. 20C shows mean inward and outward I_{Cl,SWELL} current densities at +100 and −100 mV from non-T2D (n=3 cells) and T2D (n=6 cells) mouse cells.

FIG. 20D shows mean inward and outward I_{Cl,SWELL} current densities at +100 and −100 mV from non-T2D (n=6 cells) and T2D (n=22 cells) human cells.

FIG. 20E shows mean inward and outward I_{Cl,SWELL} current densities at +100 and −100 mV from adipocytes isolated from visceral fat of lean # (n=7 cells), obese non-T2D # (n=13 cells) and T2D patients (n=5 cells). #Data from lean and obese non-T2D adipocytes replotted from previously reported data in Zhang et al., 2017 for purposes of comparison.

FIG. 20F shows a western blot of SWELL1 protein expression in inguinal adipose tissue isolated from polygenic-T2D KKAY mice compared to the parental control strain KKAa (n=5 each).

FIG. 20G shows a western blot comparing SWELL1 protein expression in visceral adipose tissue isolated from lean, obese non-T2D, and obese T2D patients, respectively.

FIG. 20H shows a western blot of SWELL1 protein isolated from cadaveric islets of non-T2D and T2D donors (n=3 each).

FIG. 21A shows western blots detecting SWELL1, pAKT2, AKT2 and -actin with 0 and 10 nM insulin stimulation for 15 min in wildtype (WT, black), SWELL1 knockout (KO, light grey) and adenoviral overexpression of SWELL1 in KO (KO+SWELL 1 O/E, dark grey) 3T3-F442A adipocytes (top). The corresponding densitometric ratio for pAKT2/-actin are shown below (n=3 independent experiments for each condition). All densitometries are normalized to values of 0 nM insulin of WT 3T3-F442A pre-adipocytes except for bottom panel. Data are represented as Mean±SEM. Two-tailed unpaired t-test was used where *, ** and *** represents p<0.05, p<0.01 and p<0.001 respectively.

FIG. 21B shows mean inward and outward current densities at +100 and −100 mV from WT (black, n=5 cells), KO (light grey, n=4 cells) and KO+SWELL 1 O/E (dark grey, n=4 cells) 3T3-F442A preadipocytes. Data are represented as Mean±SEM. Two-tailed unpaired t-test was used where *, ** and *** represents p<0.05, p<0.01 and p<0.001 respectively.

FIG. 21C shows a western blot comparing levels of SWELL1, pAKT2, AKT2 and -actin (c) with 0 and 10 nM insulin stimulation in wildtype (WT, black) and SWELL1 overexpression in WT (WT+SWELL1 O/E, grey) 3T3-F442A adipocytes (n=6 independent experiments for each condition). The corresponding densitometric ratio for pAKT2/-actin and total AKT2 is shown below All densitometries are normalized to values of 0 nM insulin of WT 3T3-F442A pre-adipocytes except for bottom panel. Data are represented as Mean±SEM. Two-tailed unpaired t-test was used where *, ** and *** represents p<0.05, p<0.01 and p<0.001 respectively.

FIG. 21D shows a western blot comparing levels of pAS160, AS160 and -actin with 0 and 10 nM insulin stimulation in wildtype (WT, black) and SWELL1 overexpression in WT (WT+SWELL1 O/E, grey) 3T3-F442A adipocytes (n=6 independent experiments for each condition). The corresponding densitometric ratio and pAS160/-actin (right top) and total AS160 (right bottom) are also shown. All densitometries are normalized to values of 0 nM insulin of WT 3T3-F442A pre-adipocytes except for bottom panel. Data are represented as Mean±SEM. Two-tailed unpaired t-test was where *, ** and *** represents p<0.05, p<0.01 and p<0.001 respectively.

FIG. 21E shows a cartoon model of homomeric mouse LRRC8a/SWELL 1 derived from cryo-electron microscopy (EM) and x-ray crystallography structure (PDB ID: 6G90#). SN-401/DCPIB bound in the pore region derived from DCPIB bound SWELL1 cryo-EM structure (PDB ID: 6NZW$; shown as a dimer for descriptive purpose) and SN-401 chemical structure (top).

FIG. 21F shows I_{Cl,SWELL} inward and outward current over time upon hypotonic (210 mOsm) stimulation and subsequent inhibition by 10 μM SN-401 in a HEK-293 cell.

FIG. 21G shows western blots detecting SWELL1, pAKT2 and -actin with 0, 3 and 10 nM insulin-stimulation in WT 3T3-F442A preadipocytes (n=2 independent experiments for each condition, top) and corresponding densitometric ratio for SWELL1/-actin and pAKT2/-actin (bottom). All densitometries are normalized to values of 0 nM insulin of WT 3T3-F442A pre-adipocytes except for bottom panel. Data are represented as Mean±SEM. Two-tailed unpaired t-test was used where *, ** and *** represents p<0.05, p<0.01 and p<0.001 respectively.

FIG. 21H shows western blots detecting SWELL1, pAKT2, AKT2 and -actin with 0 and 10 nM insulin in WT and KO 3T3-F442A adipocytes (n=6 independent experiments for each condition).

FIG. 21I shows the corresponding densitometric ratio for SWELL1/-actin from FIG. 21H. All densitometries are normalized to values of 0 nM insulin of WT 3T3-F442A pre-adipocytes except for bottom panel. Data are represented as Mean±SEM. Two-tailed unpaired t-test was used where *, ** and *** represents p<0.05, p<0.01 and p<0.001 respectively.

FIG. 21J shows the corresponding densitometric ratio for pAKT/actin (top) and pAKT2/AKT2 (bottom) from FIG. 21H. The densitometries in the top panel are normalized to values of 0 nM insulin of WT 3T3-F442A pre-adipocytes. The pAKT2/AKT2 normalization in the bottom panel was done to 0 nM insulin for WT and 0 nM insulin for KO values respectively due to the differential expression of total AKT2 in WT and KO. #Deneka et al. (2018) and $Kern et al. (2019). Data are represented as Mean±SEM. Two-tailed unpaired t-test was used where *, ** and *** represents p<0.05, p<0.01 and p<0.001 respectively.

FIG. 21K shows a western plot of the expression of pAS160, AS160 and -actin with 0 and 10 nM insulin-stimulation in WT 3T3-F442A adipocytes (n=3 independent experiments for each condition, left) and the corresponding densitometric ratio of pAS160/AS160 (right) incubated in either vehicle or 10 μM SN-401 for 96 h. All densitometries are normalized to values of 0 nM insulin of WT 3T3-F442A pre-adipocytes except for bottom panel. Data are represented as Mean±SEM. Two-tailed unpaired t-test was used where *, ** and *** represents p<0.05, p<0.01 and p<0.001 respectively.

FIG. 22A shows chemical structures of SN-401, SN-403, SN-406, SN-407, SN071 and SN072.

FIG. 22B shows I_{Cl,SWELL} inward and outward current over time upon hypotonic (210 mOsm) stimulation and subsequent inhibition with 7 μM SN-401/SN-406 or 10 μM SN071/SN072 in HEK-293 cells.

FIG. 22C shows mean of percentage of maximum outward current blocked by SN-401 (n=6), SN-403 (n=3), SN-406 (n=4), SN071 (n=3) and SN072 (n=3) at 10 μM (left) and by SN-403 (n=3), SN-406 (n=5) and SN-407 (n=3) at 7 μM (right) in HEK-293 cells, respectively. Mean presented±SEM. Two-tailed unpaired t-test was used. *, **, and *** represents p<0.05, p<0.01 and p<0.001, respectively.

FIG. 22D shows a side view without protein surface (i) and top view with protein surface of SN-401 (ii) (pink sticks) occupying the pore as resolved in the cryo-EM structure adapted from RCSB PDB: 6NZZ; SN-401 carboxylate group interacts electrostatically with the guanidine group of R103 residues (cyan sticks), SN-401 cyclopentyl and butyl group do not interact with any channel residues.

FIG. 22E shows poses generated for SN-401 by docking into PDB 6NZZ using Molecular Operating Environment 2016 (MOE) software package. SN-401 are depicted as yellow sticks and R103, D102 and L101 are depicted as cyan sticks with or without molecular surface. Panel (i) shows a side view without protein surface and panel (ii) shows a top view with protein surface of top binding pose of SN-401; SN-401 carboxylate groups interacts with R103 residue guanidine groups, the SN-401 cyclopentyl group occupies a shallow hydrophobic cleft at the interface of two monomers formed by SWELL1 D102 and L101.

FIG. 22F shows poses generated for SN071 by docking into PDB 6NZZ using Molecular Operating Environment 2016 (MOE) software package. SN071 is depicted as orange sticks and R103, D102 and L101 are depicted as cyan sticks with or without molecular surface; Panel (i) shows the top view of first binding pose of SN071 showing potential electrostatic interaction with R103 (dotted circle) but unable to reach into and occupy the hydrophobic cleft (black arrow); Panel (ii) shows the top view of second pose for SN071 with the cyclopentyl group occupying the hydrophobic cleft (dotted circle) but the carboxylate group unable to reach and interact with R103 (black arrow).

FIG. 22G shows poses generated for SN-406 by docking into PDB 6NZZ using Molecular Operating Environment 2016 (MOE) software package. SN-406 is depicted as yellow sticks and R103, D102 and L101 are depicted as cyan sticks with or without molecular surface; Panel (i) shows the top view of best binding pose of SN-406; the carboxylate group interacts with R103, cyclopentyl group occupies the hydrophobic cleft and the alkyl side chain SN-406 interacts with the alkyl side chain of R103; Panel (ii) shows SN-406 depicted as yellow space filled model.

FIG. 23A shows western blots detecting SWELL1 and -actin in 3T3-F442A adipocytes treated with vehicle (n=8), SN-401 (n=10), SN-406 (n=6), or SN072 (n=6) (SWELL1-inactive SN-401 congener) at 10 μM for 96 h and corresponding densitometric ratio for SWELL1/-actin. Data are represented as mean±SEM. Two-tailed unpaired t-test was used (compared to vehicle). *, ** and *** represents p<0.05, p<0.01 and p<0.001 respectively.

FIG. 23B shows western blots detecting SWELL1 and -actin in 3T3-F442A adipocytes treated with vehicle (n=6), SN-401 (n=6), SN-406 (n=3), SN071 (n=3) (inactive SN-401 congener) or SN072 (n=4) at 1 μM for 96 h and corresponding densitometric ratio for SWELL1/-actin. Data are represented as mean±SEM. Two-tailed unpaired t-test was used (compared to vehicle). *, ** and *** represents p<0.05, p<0.01 and p<0.001 respectively.

FIG. 23C shows immunostaining images demonstrating localization of endogenous SWELL1 in 3T3-F442A preadipocytes treated with vehicle (n=19), SN-401 (n=21), SN-406 (n=13 for 1 and 10 μM), or SN071 (n=9 for 1 μM and n=13 for 10 μM) at 1 or 10 μM for 48 h (Scale bar—20 μm) and corresponding quantification of SWELL1 membrane- versus cytoplasm-localized fraction. Data are represented as mean±SEM. One-way ANOVA was used (compared to vehicle). *, ** and *** represents p<0.05, p<0.01 and p<0.001 respectively.

FIG. 23D shows I_{C1.SWELL} inward and outward current over time recorded from HEK-293 cells preincubated with vehicle, SN-401, SN-406, SNO71 or SN072 at 1 μM and subsequently stimulated with hypotonic solution.

FIG. 23E shows mean outward outward lcl,swELL current densities at +100 mV measured at 7 min timepoint after hypotonic stimulation in FIG. 23D. Data are represented as mean±SEM. One-way ANOVA was used (compared to vehicle). *, ** and *** represents p<0.05, p<0.01 and p<0.001 respectively.

FIG. 23F shows I_{C1.SWELL} inward and outward current over time recorded from HEK-293 cells preincubated with vehicle, SN-401, SN-406, SNO71 or SN072 at 250 nM concentration and subsequently stimulated with hypotonic solution.

FIG. 23G shows mean outward outward lcl,swELL current densities at +100 mV measured at 7 min timepoint after hypotonic stimulation in FIG. 23F. Data are represented as mean±SEM. One-way ANOVA was used (compared to vehicle). *, ** and *** represents p<0.05, p<0.01 and p<0.001 respectively.

FIG. 23H shows western blots detecting pAKT2, AKT2 and -actin in 3T3-F442A adipocytes treated with vehicle (n=3 for 0 nM insulin, n=5 for 10 nM insulin) or 1 μM SN-401 (n=3 for 0 nM insulin, n=6 for 10 nM insulin) and corresponding densitometric ratio for pAKT2/-actin and pAKT2/AKT2. Data are represented as mean±SEM. Two-tailed unpaired t-test was used (compared to vehicle). *, ** and *** represents p<0.05, p<0.01 and p<0.001 respectively.

FIG. 23I shows western blots detecting SWELL1 and -actin in 3T3-F442A adipocytes treated with vehicle, 1 mM palmitate+vehicle, 1 mM palmitate+10 μM SN-401, 1 mM palmitate+10 μM SN-406, 1 mM palmitate+10 μM SN072 (n=3 in each condition) and corresponding densitometric ratio for SWELL1/-actin. Data are represented as mean±SEM. Two-tailed unpaired t-test was used (compared to vehicle). *, ** and *** represents p<0.05, p<0.01 and p<0.001 respectively.

FIG. 24A shows western blots detecting SWELL1 protein in visceral fat of C57BL/6 mice on high-fat diet (HFD) for 21 weeks and treated with either vehicle or SN-401 (5 mg/kg i.p.) and the corresponding densitometric ratios for SWELL1/-actin (right) (n=6 mice in each group). Mean presented±SEM. Two-tailed unpaired t-test. *, ** and *** representing p<0.05, p<0.01 and p<0.001, respectively

FIG. 24B shows western blots comparing SWELL1 protein expression in inguinal adipose tissue of a polygenic-T2D KKAY mouse treated with SN-401 (5 mg/kg i.p daily×14 days) compared to untreated control KKAa and wild-type C57BL/6 mice.

FIG. 24C shows glucose tolerance test (GTT) and insulin tolerance test (ITT) of C57BL/6 mice on HFD for 8 weeks treated with either vehicle or SN-401 (5 mg/kg i.p) for 10 days (n=7 mice in each group). Mean presented±SEM. Two-way ANOVA was used (p-value in bottom corner of graph). *, ** and *** representing p<0.05, p<0.01 and p<0.001, respectively.

FIG. 24D shows fasting glucose levels (of T2D KKAY mice (n=6) and its control strain KKAa (n=3) compared pre- and post-SN-401 (5 mg/kg i.p) treatment for 4 days, respectively. Mean presented±SEM. Paired t-test. *, ** and *** representing p<0.05, p<0.01 and p<0.001, respectively

FIG. 24E shows fasting glucose levels (d), GTT (e) and ITT (f) of T2D KKAY mice (n=6) and its control strain KKAa (n=3) compared pre- and post-SN-401 (5 mg/kg i.p) treatment for 4 days, respectively. Mean presented±SEM. Two-way ANOVA was used (p-value in bottom corner of graph). *, ** and *** representing p<0.05, p<0.01 and p<0.001, respectively.

FIG. 24F shows fasting glucose levels (d), GTT (e) and ITT (f) of T2D KKAY mice (n=6) and its control strain KKAa (n=3) compared pre- and post-SN-401 (5 mg/kg i.p) treatment for 4 days, respectively. Two-way ANOVA was used (p-value in bottom corner of graph). *, ** and *** representing p<0.05, p<0.01 and p<0.001, respectively.

FIG. 24G shows fasting glucose levels (g) of regular chow-diet fed (RC), lean mice treated with either vehicle or SN-401 (5 mg/kg i.p) for 6 days (n=6 in each group). Mean presented±SEM. Two-tailed unpaired t-test. *, ** and *** representing p<0.05, p<0.01 and p<0.001, respectively.

FIG. 24H shows corresponding GTT to the fasting glucose levels in FIG. 24G of regular chow-diet fed (RC), lean mice treated with either vehicle or SN-401 (5 mg/kg i.p) for 6 days (n=6 in each group).

FIG. 24I shows fasting glucose levels of HFD-T2D mice treated with either vehicle or SN-401 (5 mg/kg i.p). Mean presented±SEM. Two-tailed unpaired t-test. *, ** and *** representing p<0.05, p<0.01 and p<0.001, respectively

FIG. 24J shows GTT (16 weeks HFD, 4 days treatment) and ITT (18 weeks HFD, 4 days treatment) of HFD-T2D mice treated with either vehicle or SN-401 (5 mg/kg i.p). Mean presented±SEM. Two-way ANOVA was used (p-value in bottom corner of graph). *, ** and *** representing p<0.05, p<0.01 and p<0.001, respectively.

FIG. 24K shows relative insulin secretion in plasma of HFD-T2D mice (18 weeks HFD, 4 days treatment) after i.p. glucose (0.75 g/kg BW) treated with either vehicle (n=3) or SN-401 (n=4, 5 mg/kg i.p).

FIG. 24L shows glucose stimulated insulin secretion (GSIS) perifusion assay from islets isolated from HFD-T2D mouse (21 week timepoint) treated with either vehicle (n=3 mice, and 3 experimental replicates) or SN-401 (n=3 mice, and 2 experimental replicates, 5 mg/kg i.p) and their corresponding area under the curve (AUC) comparisons, respectively, on the right. Mean presented±SEM. Two-tailed unpaired t-test. *, ** and *** representing p<0.05, p<0.01 and p<0.001, respectively

FIG. 24M shows glucose stimulated insulin secretion (GSIS) perifusion assay from islets isolated from polygenic-T2D KKAY mouse treated with either vehicle or SN-401 (5 mg/kg i.p for 6 days, n=3 mice in each group, 3 experimental replicates), and their corresponding area under the curve (AUC) comparisons, respectively, on the right. Mean presented±SEM. Two-tailed unpaired t-test. *, ** and *** representing p<0.05, p<0.01 and p<0.001, respectively.

FIG. 25A shows mean glucose-infusion rate during euglycemic hyperinsulinemic clamps of polygenic T2D KKAY mice treated with vehicle (n=7) or SN-401 (n=8) for 4 days. Mean presented±SEM. Two-tailed unpaired t-test. Statistical significance is denoted by *, ** and *** representing p<0.05, p<0.01 and p<0.001, respectively.

FIG. 25B shows hepatic glucose production at baseline and during euglycemic hyperinsulinemic clamp of T2D KKAY mice treated with vehicle or SN-401 (n=9 in each group). Mean presented±SEM. Two-tailed unpaired t-test. Statistical significance is denoted by *, ** and *** representing p<0.05, p<0.01 and p<0.001, respectively.

FIG. 25C shows glucose uptake determined from 2-deoxyglucose (2-DG) uptake in inguinal while adipose tissue (iWAT) and gonadal white adipose tissue (gWAT) and heart during traced clamp of T2D KKAY mice treated with vehicle or SN-401 (n=9 in each group). Mean presented±SEM. Two-tailed unpaired t-test. Statistical significance is denoted by *, ** and *** representing p<0.05, p<0.01 and p<0.001, respectively.

FIG. 25D shows Glucose uptake into glycogen determined from 2-DG uptake in liver (n=9 for vehicle and n=8 for SN-401), adipose (iWAT, n=7 vehicle and n=6 SN-401) and gastrocnemius muscle (n=7 vehicle and n=6 SN-401) during clamp of T2D KKAY mice. Mean presented±SEM. Two-tailed unpaired t-test. Statistical significance is denoted by *, ** and *** representing p<0.05, p<0.01 and p<0.001, respectively.

FIG. 25E shows a schematic representation of treatment protocol of C57BL/6 mice injected with either vehicle or SN-401 (n=6 in each group) during HFD-feeding.

FIG. 25F shows liver mass (left) and normalized ratio to body mass (right) of HFD-T2D mice following treatment with either vehicle or SN-401 (5 mg/kg i.p.). Mean presented±SEM. Two-tailed unpaired t-test. Statistical significance is denoted by *, ** and *** representing p<0.05, p<0.01 and p<0.001, respectively.

FIG. 25G shows corresponding hematoxylin- and eosin-stained liver sections. Scale bar-100 μm.

FIG. 25H shows liver triglycerides (6 mice in each group). Mean presented±SEM. Two-tailed unpaired t-test. Statistical significance is denoted by *, ** and *** representing p<0.05, p<0.01 and p<0.001, respectively.

FIG. 25I shows histologic scoring for steatosis, lobular inflammation, hepatocyte damage (ballooning), and NAFLD-activity score (NAS), which integrates scores for steatosis, inflammation, and ballooning. Mean presented±SEM. Two-tailed unpaired t-test. Statistical significance is denoted by *, ** and *** representing p<0.05, p<0.01 and p<0.001, respectively.

FIG. 26A shows fasting glucose levels, GTT and its corresponding area under the curve (AUC) of 8 week HFD-fed mice treated with either SWELL1-inactive SN-071 or SWELL1-active SN-403 (5 mg/kg i.p) for 4 days (n=5 in each group). Data are represented as mean±SEM. Two-way ANOVA for GTT. Two-tailed unpaired t-test was used for FG, GTT AUC, GSIS AUC and HOMA-IR. Statistical significance is denoted by *, ** and *** representing p<0.05, p<0.01 and p<0.001 respectively.

FIG. 26B shows fasting glucose levels, GTT and its corresponding AUC of 12 weeks HFD-fed mice pre- and post-treatment of SN-406 (5 mg/kg i.p) for 4 days (n=5 in each group). Two-way ANOVA for GTT. Paired t-test for FG and GTT AUC. Statistical significance is denoted by *, ** and *** representing p<0.05, p<0.01 and p<0.001 respectively.

FIG. 26C shows GTT and corresponding AUC of 12 weeks HFD-fed mice treated with either SWELL1-inactive SN-071 or SWELL1-active SN-406 (5 mg/kg i.p) for 4 days (n=7 in each group). Data are represented as mean±SEM. Two-tailed unpaired t-test was used for FG, GTT AUC, GSIS AUC and HOMA-IR. Two-way ANOVA in a-c and f for GTT Statistical significance is denoted by *, ** and *** representing p<0.05, p<0.01 and p<0.001 respectively.

FIG. 26D shows the corresponding HOMA-IR index to the data shown in FIG. 26C. Data are represented as mean±SEM. Two-tailed unpaired t-test was used for FG, GTT AUC, GSIS AUC and HOMA-IR. Statistical significance is denoted by *, ** and *** representing p<0.05, p<0.01 and p<0.001 respectively.

FIG. 26E shows glucose-stimulated insulin secretion (GSIS) perifusion assay of islets isolated from mice in 26C. Data are represented as mean±SEM. Two-tailed unpaired t-test was used for FG, GTT AUC, GSIS AUC and HOMA-IR. Statistical significance is denoted by *, ** and *** representing p<0.05, p<0.01 and p<0.001 respectively.

FIG. 26F shows GTT and corresponding AUC of polygenic-T2D KKAY mice treated with either SWELL1-inactive SN-071 (n=5) or SWELL1-active SN-407 (n=6) (5 mg/kg i.p) for 4 days. Data are represented as mean±SEM. Two-tailed unpaired t-test was used for FG, GTT AUC, GSIS AUC and HOMA-IR. Two-way ANOVA in a-c and f for GTT. Statistical significance is denoted by *, ** and *** representing p<0.05, p<0.01 and p<0.001 respectively.

FIG. 26G shows glucose-stimulated insulin secretion (GSIS) perifusion assay from islets isolated from mice in 26F. Data are represented as mean±SEM. Two-tailed unpaired t-test was used for FG, GTT AUC, GSIS AUC and HOMA-IR. Statistical significance is denoted by *, ** and *** representing p<0.05, p<0.01 and p<0.001 respectively.

FIG. 27A shows current-voltage plots of lcl,swELL measured in 3T3-F442A preadipocytes WT at baseline (iso, black trace) and hypotonic (hypo, red trace) stimulation respectively.

FIG. 27B shows current-voltage plots of lcl,swELL measured in 3T3-F442A preadipocytes KO at baseline (iso, black trace) and hypotonic (hypo, red trace) stimulation respectively.

FIG. 27C shows adenoviral overexpression of SWELL1 in KO (KO+SWELL 1 O/E) at baseline (iso, black trace) and hypotonic (hypo, red trace) stimulation respectively.

FIG. 27D shows immunostaining images demonstrating localization of endogenous SWELL1 or overexpressed SWELL1 with anti-Flag or anti-SWELL1 antibody (Scale bar—20 μm).

FIG. 27E shows validation of SWELL1 antibody in WT 3T3-F442A compared to SWELL1 KO pre-adipocytes (Scale bar—20 μm), revealing a punctate pattern of endogenous SWELL1 localization (inset).

FIG. 28 shows relative mRNA expression of LRRC8 family members to GAPDH assessed by qPCR (n=3 each) for 3T3 F-442A preadipocytes treated with vehicle or SN-401 at 10 μM for 96 h. Data are represented as mean±SEM. Two-tailed unpaired t-test was used where *, ** and *** represents p<0.05, p<0.01 and p<0.001 respectively.

FIG. 29A shows chemical structures (top) of SN-401/DCPIBand lcl,swELL inward and outward current over time (bottom) upon hypotonic (210 mOsm) stimulation and subsequent inhibition by 7 μM SN-401 in HEK-293 cell.

FIG. 29B shows the chemical structure of SN-403 and lcl,swELL inward and outward current over time (bottom) upon hypotonic (210 mOsm) stimulation and subsequent inhibition by 7 μM SN-403 in HEK-293 cell.

FIG. 29C shows the chemical structure of SN-407 and lcl,swELL inward and outward current over time (bottom) upon hypotonic (210 mOsm) stimulation and subsequent inhibition by 7 μM SN-407 in HEK-293 cells.

FIG. 29D shows that binding poses for SN072 reveal that the carboxylate group can reach and electrostatically interact with R103 but in the absence of the butyl group cannot orient the cyclopentyl ring to occupy the hydrophobic cleft without introducing excessive structural strain on the carbon connecting the core with the cyclopentyl ring.

FIG. 29E shows alternative view of best binding pose of SN-406; the carboxylate group interacts with R103, cyclopentyl group occupies the hydrophobic cleft and the alkyl side chain SN-406 interacts with the alkyl side chain of R103.

FIG. 29F panel (i) shows side view without protein surface and panel (ii) shows top view with protein surface of top binding pose of SN-403. The carboxylate groups interacts with guanidine group of R103 residues (solid circle), the cyclopentyl group occupies a shallow hydrophobic cleft at the interface of two monomers formed by D102 and L101 (dotted circle).

FIG. 29G shows (i) side view without protein surface and (ii) top view with protein surface of top binding pose of SN-407; the carboxylate group interacts with R103 (solid circle), cyclopentyl group occupies the hydrophobic cleft (dotted circle) and the alkyl side chain SN-407 interacts with the alkyl side chain of R103.

FIG. 29H shows I_{Cl,SWELL} inward and outward current over time upon hypotonic stimulation in WT (left) and R103E mutant overexpressed (right) HEK-293 cells, respectively and subsequent inhibition by 7 μM SN-406.

FIG. 29I shows mean of percentage of maximum outward current blocked by SN-406 at 10 μM (left) and 7 μM (right) in WT (n=4 at 10 μM and n=5 at 7 μM) and R103E mutant (n=5 at 10 μM and n=6 at 7 μM) overexpressed in HEK-293 cells respectively. Data are represented as mean±SEM. Two-tailed unpaired t-test was used where *, ** and *** represents p<0.05, p<0.01 and p<0.001 respectively.

FIG. 30 shows immunostaining images demonstrating localization of endogenous SWELL1 in WT 3T3-F442A preadipocytes treated with vehicle or SN-401, SN-406, and SNO71 at 1 and 10 μM for 48 h (Scale bar—20 μm).

FIG. 31A shows fasting glucose levels of C57BL/6 lean mice on regular-chow diet treated with either vehicle or SN-401 (5 mg/kg i.p) for 10 days (n=7 males in each group). Two-tailed unpaired t-test was used for FG and AUC.

FIG. 31B shows GTT of C57BL/6 lean mice on regular-chow diet treated with either vehicle or SN-401 (5 mg/kg i.p) for 10 days (n=7 males in each group). Data are represented as mean±SEM. Two-way ANOVA was used for GTTs and ITTs. Statistical significance is denoted by *, ** and *** representing p<0.05, p<0.01 and p<0.001 respectively and ‘ns’ indicates the difference was not significant.

FIG. 31C shows ITT of C57BL/6 lean mice on regular-chow diet treated with either vehicle or SN-401 (5 mg/kg i.p) for 10 days (n=7 males in each group). Data are represented as mean±SEM. Two-way ANOVA was used for b-d, and i for GTTs and ITTs. Statistical significance is denoted by *, ** and *** representing p<0.05, p<0.01 and p<0.001 respectively and ‘ns’ indicates the difference was not significant.

FIG. 31D shows GTT of HFD-T2D mice (8 weeks HFD) treated with either vehicle (n=5 males) or SN-401 (5 mg/kg i.p, n=4 males) for 8 weeks. Data are represented as mean±SEM. Two-way ANOVA was used for GTTs and ITTs. Statistical significance is denoted by *, ** and *** representing p<0.05, p<0.01 and p<0.001 respectively and ‘ns’ indicates the difference was not significant.

FIG. 31E shows in vivo pharmacokinetics of SN-401 administered at 5 mg/kg intraperitoneally (i.p).

FIG. 31F shows in vivo pharmacokinetics of SN-406 administered at 5 mg/kg intraperitoneally (i.p).

FIG. 31G shows in vivo pharmacokinetics of SN-401 administered at 5 mg/kg by oral gavage (p.o).

FIG. 31H shows in vivo pharmacokinetics of SN-406 administered at 5 mg/kg by oral gavage (p.o).

FIG. 31I shows fasting glucose levels, GTT and AUC of HFD-T2D mice (10 weeks HFD) treated with either vehicle (n=6 males) or SN-401 (5 mg/kg p.o, n=7 males) for 5 days. Data are represented as mean±SEM. Two-way ANOVA was used for b-d, and i for GTTs and ITTs. Two-tailed unpaired t-test was used for FG and AUC. Statistical significance is denoted by *, ** and *** representing p<0.05, p<0.01 and p<0.001 respectively and ‘ns’ indicates the difference was not significant.

FIG. 32A shows glucose uptake determined from 2-DG uptake in brown fat, extensor digitorum longus (EDL), soleus and gastrocnemius muscles harvested under clamp for KKAY mice treated with vehicle or SN-401 (n=9 in each group, 5 mg/kg i.p) for 4 days. Data are represented as mean±SEM. Two-tailed unpaired t-test was used for the analysis. ‘ns’ indicates the difference was not significant.

FIG. 32B shows images of hematoxylin and eosin stained liver histology sections of HFD-T2D mice treated with either vehicle or SN-401 (5 mg/kg i.p). Scale—(10×: 100 μm and 20×: 50 μm).

FIG. 33A shows western blots from WT and SWELL1 KO C2C12 (left) and primary myotubes (right).

FIG. 33B shows current-voltage curves from WT and SWELL1 KO C2C12 myoblast measured during a voltage-ramp from −100 to +100 mV+/−isotonic and hypotonic (210 mOsm) solution.

FIG. 33C shows bright field merged with fluorescence images of differentiated WT and SWELL1 KO C2C12 myotubes (left, middle) and skeletal muscle primary cells (right). DAPI stains nuclei blue (middle). Red is mCherry reporter fluorescence from adenoviral transduction. Scale bar: 100 Mean myotube surface area measured from WT (n=21) and SWELL1 KO (n=21) C2C12 myotubes (left), and WT (n=22) and SWELL1 KO (n=15) primary skeletal myotubes (right). Fusion index (% multinucleated cells) measured from WT (n=5 fields) and SWELL1 KO (n=5 fields) C2C12 (shown below the representative image).

FIG. 33D shows a heatmap of top 17 differentially expressed genes in WT versus SWELL1 KO C2C12 myotubes derived from RNA sequencing.

FIG. 33E shows Reads Per Kilobase Million for select myogenic differentiation genes (n=3, each).

FIG. 33F shows IPA canonical pathway analysis of genes significantly regulated in SWELL1 KO C2C12 myotubes in comparison to WT. n=3 for each group. For analysis with IPA, FPKM cutoffs of 1.5, fold change of >1.5, and false discovery rate <0.05 were utilized for significantly differentially regulated genes. Statistical significance between the indicated values were calculated using a two-tailed Student's t-test. Error bars represent mean±s.e.m. *, P<0.05, **, P<0.01, ***, P<0.001, ****, P<0.0001. n=3, independent experiments.

FIG. 34A shows western blots of SWELL1, pAKT2, AKT2, pAS160, AS160, pAMPK, AMPK, pFoxO1, FoxO1 and β-actin in WT and SWELL1 KO C2C12 myotubes upon insulin-stimulation (10 nM).

FIG. 34B shows western blots of SWELL1, AKT2, pAKT2, pAS160, pAKT1, AKT1 and GAPDH in WT (Ad-CMV-mCherry) and SWELL1 KO (Ad-CMV-Cre-mCherry) primary skeletal muscle myotubes following insulin-stimulation (10 nM).

FIG. 34C shows densitometric quantification of proteins depicted on western blots normalized to β-actin. Statistical significance between the indicated values were calculated using a two-tailed Student's t-test. Error bars represent mean±s.e.m. *, P<0.05, **, P<0.01, ***, P<0.001, ****, P<0.0001. n=3, independent experiments.

FIG. 34D shows densitometric quantification of proteins depicted on western blots normalized to GAPDH. Statistical significance between the indicated values were calculated using a two-tailed Student's t-test. Error bars represent mean±s.e.m. *, P<0.05, **, P<0.01, ***, P<0.001, ****, P<0.0001. n=3, independent experiments.

FIG. 34E shows gene expression analysis of insulin signaling associated genes AKT2, FOXO3, FOXO4, FOXO6 and GLUT4 in WT and SWELL1 KO C2C12 myotubes. Statistical significance between the indicated values were calculated using a two-tailed Student's t-test. Error bars represent mean±s.e.m. *, P<0.05, **, P<0.01, ***, P<0.001, ****, P<0.0001. n=3, independent experiments.

FIG. 35A shows bright-field image of differentiated WT, SWELL1 KO and SWELL1 KO+SWELL1 O/E C2C12 myotubes. Scale bar: 100 μm.

FIG. 35B shows quantification of mean myotube surface areas in WT (n=35), SWELL1 KO C2C12 (n=26) and SWELL1 KO+SWELL1 O/E C2C12 (n=45) cells.

Statistical significance between the indicated group were calculated with one-way Anova, Tukey's multiple comparisons test. Error bars represent mean±s.e.m. *, P<0.05, **, P<0.01, ***, P<0.001, ****, P<0.0001. n=3, independent experiments.

FIG. 35C shows western blots of SWELL1, AKT2, pAKT2, pAS160, pAKT1, AKT1, pP70S6K, P70S6K, pS6K, pERK1/2, ERK1/2, β-actin and GAPDH from WT, SWELL1 KO and SWELL1 KO+SWELL1 O/E C2C12 myotubes.

FIG. 35D shows densitometric quantification of proteins depicted on western blots normalized to β-actin and GAPDH respectively. Statistical significance between the indicated group were calculated with one-way Anova, Tukey's multiple comparisons test. Error bars represent mean±s.e.m. *, P<0.05, **, P<0.01, ***, P<0.001, ****, P<0.0001. n=3, independent experiments.

FIG. 36A shows a western blot of SWELL1, AKT2, pAKT2, pAKT1, pAS160, pERK1/2, ERK1/2 and β-actin in WT and SWELL1 KO myotube in response to 15 minutes of 0% and 5% static stretch.

FIG. 36B shows densitometric quantification of each signaling protein relative to β-actin. Statistical significance between the indicated group calculated with one-way Anova, Tukey's multiple comparisons test. Error bars represent mean±s.e.m. *, P<0.05, **, P<0.01, ***, P<0.001, ****, P<0.0001. n=3, independent experiments.

FIG. 37A shows SWELL1-3×Flag over expressed in C2C12 cells followed by immunoprecipitation (IP) with Flag antibody. Western blot of Flag, SWELL1, GRB2 and GAPDH. IgG serves as a negative control.

FIG. 37B shows a western blot of GRB2 to validate GRB2 knock down efficiency in SWELL1 KO/GRB2 knock-down (Ad-shGRB2-GFP) compared to WT C2C12 (Ad-shSCR-GFP) and SWELL1 KO (Ad-shSCR-GFP). Densitometric quantification of GRB2 knock-down relative to GAPDH (right). Statistical significance between the indicated group were calculated with one-way Anova, Tukey's multiple comparisons test. Error bars represent mean±s.e.m. *, P<0.05, **, P<0.01, ***, P<0.001, ****, P<0.0001. n=3, independent experiments.

FIG. 37C shows a fluorescence image of WT C2C12/shSCR-GFP, SWELL1 KO/shSCR-GFP and SWELL1 KO/shGRB2-GFP myotubes. Scale bar: 100 μm.

FIG. 37D shows a quantification of mean myotube area of WT C2C12/shSCR-GFP (n=25), SWELL1 KO/shSCR-GFP (n=28) and SWELL1 KO/shGRB2-GFP (n=24). Statistical significance between the indicated group were calculated with one-way Anova, Tukey's multiple comparisons test. Error bars represent mean±s.e.m. *, P<0.05, **, P<0.01, ***, P<0.001, ****, P<0.0001. n=3, independent experiments.

FIG. 37E shows relative mRNA expression of selected myogenic differentiation genes in SWELL1 KO/shSCR and SWELL1 KO/shGRB2 compared to WT C2C12/shSCR (n=3 each), and of SWELL1 KO/shGRB2 compared to SWELL1 KO/shSCR. Statistical significance between the indicated group were calculated with one-way Anova, Tukey's multiple comparisons test. Error bars represent mean±s.e.m. *, P<0.05, **, P<0.01, ***, P<0.001, ****, P<0.0001. n=3, independent experiments.

FIG. 37F shows fold change of mRNA's in KO shGRB2 relative to KO cells with preserved GRB2 expression.

FIG. 38A shows a schematic representation of Cre-mediated recombination of loxP sites flanking Exon 3 using muscle-specific Myf5-Cre mice to generate skeletal muscle targeted SWELL1 KO mice.

FIG. 38B shows a western blot of gastrocnemius muscle protein isolated from of WT and Myf5-Cre;SWELL1fl/fl (Myf5 KO) mice. Liver sample from Myf5 KO and C2C12 cell lysates used as a positive control for SWELL1. Coomassie gel, below, serves as loading control for skeletal muscle protein. Densitometric quantification for SWELL1 deletion in skeletal muscle of Myf5 KO mice (n=3) compared to WT (n=3; SWELL1fl/fl) (right). Statistical significance between the indicated values were calculated using a two-tailed Student's t-test. Error bars represent mean±s.e.m. *, P<0.05, **, P<0.01, ***, P<0.001, ****, P<0.0001.

FIG. 38C shows NMR measurement of lean mass (%) and absolute fat mass of WT (n=11) and Myf5 KO (n=7) mice. Statistical significance between the indicated values were calculated using a two-tailed Student's t-test. Error bars represent mean±s.e.m. *, P<0.05, **, P<0.01, ***, P<0.001, ****, P<0.0001.

FIG. 38D shows absolute muscle mass of muscle groups freshly isolated from WT (n=3) and Myf5 KO (n=4). Statistical significance between the indicated values were calculated using a two-tailed Student's t-test. Error bars represent mean±s.e.m. *, P<0.05, **, P<0.01, ***, P<0.001, ****, P<0.0001.

FIG. 38E shows haematoxylin and eosin staining of tibialis muscle of WT and Myf5 KO mice fed on regular chow diet for 28 weeks (above). Scale bar: 100 μm. Below, ImageJ converted image highlights distinct surface boundaries of myotubes. Inset, enlarged image shows smaller fiber size in Myf5 KO muscle tissue. Quantification of average cross-sectional area of muscle fiber of WT (n=300) and Myf5 KO (n=300) mice from 10-12 different view field images (right). Statistical significance between the indicated values were calculated using a two-tailed Student's t-test. Error bars represent mean±s.e.m. *, P<0.05, **, P<0.01, ***, P<0.001, ****, P<0.0001.

FIG. 39A shows exercise treadmill tolerance test for Myf5 KO mice (n=14) compared to WT littermates (n=15). Statistical significance between the indicated values were calculated using a two-tailed Student's t-test. Error bars represent mean±s.e.m. *, P<0.05, **, P<0.01, ***, P<0.001, ****, P<0.0001.

FIG. 39B shows hang times on inversion testing of Myf5 KO (n=8) and WT (n=9) mice. Statistical significance between the indicated values were calculated using a two-tailed Student's t-test. Error bars represent mean±s.e.m. *, P<0.05, **, P<0.01, ***, P<0.001, ****, P<0.0001.

FIG. 39C shows ex-vivo isometric peak tetanic tension of isolated soleus muscle from Myf5 KO (n=7) compared to WT (n=7) mice. Statistical significance between the indicated values were calculated using a two-tailed Student's t-test. Error bars represent mean±s.e.m. *, P<0.05, **, P<0.01, ***, P<0.001, ****, P<0.0001.

FIG. 39D shows ex-vivo time to fatigue of isolated soleus muscle from Myf5 KO (n=7) compared to WT (n=7) mice. Statistical significance between the indicated values were calculated using a two-tailed Student's t-test. Error bars represent mean±s.e.m. *, P<0.05, **, P<0.01, ***, P<0.001, ****, P<0.0001.

FIG. 39E shows ex-vivo half relaxation time of isolated soleus muscle from Myf5 KO (n=7) compared to WT (n=7) mice. Statistical significance between the indicated values were calculated using a two-tailed Student's t-test. Error bars represent mean±s.e.m. *, P<0.05, **, P<0.01, ***, P<0.001, ****, P<0.0001.

FIG. 39F shows oxygen Consumption Rate (OCR) in WT and SWELL1 KO primary myotubes+/−insulin stimulation (10 nM) (n=6 independent experiments) and quantification of basal OCR, OCR post Oligomycin, OCR post FCCP and OCR post Antimycin A. Statistical significance between the indicated values were calculated using a two-tailed Student's t-test. Error bars represent mean±s.e.m. *, P<0.05, **, P<0.01, ***, P<0.001, ****, P<0.0001.

FIG. 39G shows ATP-linked respiration obtained by subtracting the OCR after oligomycin from baseline cellular OCR. Statistical significance between the indicated values were calculated using a two-tailed Student's t-test. Error bars represent mean±s.e.m. *, P<0.05, **, P<0.01, ***, P<0.001, ****, P<0.0001.

FIG. 39H shows extracellular acidification rate (ECAR) in WT and SWELL1 KO primary myotubes+/−insulin stimulation (10 nM) (n=6 independent experiments) and quantification of basal OCR, OCR post Oligomycin, OCR post FCCP and OCR post Antimycin A. Statistical significance between the indicated values were calculated using a two-tailed Student's t-test. Error bars represent mean±s.e.m. *, P<0.05, **, P<0.01, ***, P<0.001, ****, P<0.0001.

FIG. 40A shows glucose and insulin tolerance tests of mice raised on chow diet of WT (n=11) and Myf5 KO (n=10) mice. Two-way ANOVA was used (p-value in bottom corner of graph).

FIG. 40B shows NMR measurement of fat mass (%) and absolute fat mass of WT (n=11) and Myf5 KO (n=7) mice. Statistical significance test was calculated by using a two-tailed Student's t-test. Error bars represent mean±s.e.m. *, P<0.05, **, P<0.01, ***, P<0.001.

FIG. 40C shows body mass of WT (n=11) and Myf5 KO (n=7) mice on regular chow diet. Statistical significance test was calculated by using a two-tailed Student's t-test. Error bars represent mean±s.e.m. *, P<0.05, **, P<0.01, ***, P<0.001.

FIG. 40D shows glucose tolerance test of WT (n=8) and Myf5 KO (n=7) mice fed HFD for 16 weeks after 14-weeks of age. Two-way ANOVA was used for p-value in bottom corner of graph. To the right shows the corresponding area under the curve (AUC) for glucose tolerance for WT and Myf5 KO mice. Statistical significance test was calculated by using a two-tailed Student's t-test. Error bars represent mean±s.e.m. *, P<0.05, **, P<0.01, ***, P<0.001.

FIG. 40E shows insulin tolerance tests of WT (n=5) and Myf5 KO (n=4) mice fed HFD for 18 weeks after 14-weeks of age. Two-way ANOVA was used for p-value in bottom corner of graph. To the right shows the corresponding area under the curve (AUC) for insulin tolerance for WT and Myf5 KO mice. Statistical significance test was calculated by using a two-tailed Student's t-test. Error bars represent mean±s.e.m. *, P<0.05, **, P<0.01, ***, P<0.001.

FIG. 41 shows differentially expressed glucose and glycogen metabolism associated gene after RNA-seq analysis of C2C12 WT and SWELL1 KO myotube (n=3, each). Statistical significance between the indicated values were calculated using a two-tailed Student's t-test. Error bars represent mean±s.e.m. *, P<0.05, **, P<0.01, ***, P<0.001, ****, P<0.0001.

FIG. 42A shows NMR measurement of fat mass (%) and lean mass (%) of WT (n=8) and Myf5 KO (n=7) mice raised on HFD (16 weeks) after 14-weeks of age.

FIG. 42B shows body mass of WT (n=8) and Myf5 KO (n=7) mice.

FIG. 43A shows a schematic representation of Cre-mediated recombination of loxP sites flanking Exon 3 using muscle-specific Myl1-Cre mice to generate skeletal muscle targeted SWELL1 KO mice (Myl1-Cre;SWELL1fl/fl; Myl1 KO)

FIG. 43B shows a PCR band of SWELL1 recombination in Myl1 KO mice from isolated tissues.

FIG. 43C shows a glucose tolerance test of WT (n=6) and Myl1KO (n=6) mice raised on chow food diet for 14 weeks. Fasting glucose level for WT and Myl1KO mice (right). Statistical significance between the indicated values were calculated using a two-tailed Student's t-test. Error bars represent mean±s.e.m. *, P<0.05.

FIG. 43D shows an exercise treadmill tolerance test for Myl1KO (n=6) compared to WT (n=6) littermates. Statistical significance between the indicated values were calculated using a two-tailed Student's t-test. Error bars represent mean±s.e.m. *, P<0.05.

FIG. 43E shows epidymal (eWAT) and inguinal (iWAT) fat mass normalized to body mass (BM) isolated from Myl1KO (n=5) and WT (n=4) mice. Statistical significance between the indicated values were calculated using a two-tailed Student's t-test. Error bars represent mean±s.e.m. *, P<0.05.

FIG. 43F shows skeletal muscle mass normalized to body mass (BM) isolated from Myl1KO (n=5) and WT (n=4) mice. Statistical significance between the indicated values were calculated using a two-tailed Student's t-test. Error bars represent mean±s.e.m. *, P<0.05.

FIG. 43G shows body mass of Myl1KO (n=5) and WT (n=4) mice raised on regular chow diet. Statistical significance between the indicated values were calculated using a two-tailed Student's t-test. Error bars represent mean±s.e.m. *, P<0.05.

### DETAILED DESCRIPTION

The present invention is directed to various polycyclic compounds and various methods using these compounds to treat a variety of conditions in a subject in need thereof including insulin sensitivity, obesity, diabetes, nonalcoholic fatty liver disease, metabolic diseases, hypertension, stroke, vascular tone, and systemic arterial and/or pulmonary arterial blood pressure and/or blood flow. Various neurological diseases, infertility problems, muscular disorders, and immune deficiencies can also be treated with these compounds.

In various embodiments, compounds of the present invention include those of Formula (I) and salts thereof:

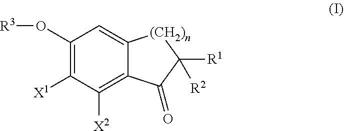

wherein

R¹ and R² are each independently hydrogen, substituted or unsubstituted alkyl, substituted or unsubstituted alkenyl, substituted or unsubstituted alkoxy, substituted or unsubstituted aryl, substituted or unsubstituted heteroaryl;

R³ is —Y—C(O)R⁴, —Z—N(R⁵)(R⁶), or —Z-A;

R⁴ is hydrogen, substituted or unsubstituted alkyl, —OR⁷, or —N(R⁸)(R⁹);

X¹ and X² are each independently hydrogen, substituted or unsubstituted alkyl, halo, —OR¹⁰, or —N(R¹¹)(R¹²);

R⁵, R⁶, R⁷, R⁸, R⁹, R¹⁰, R¹¹ and R¹² are each independently hydrogen or substituted or unsubstituted alkyl;

Y and Z are each independently a substituted or unsubstituted carbon-containing moiety having at least 2 carbon atoms;

A is a substituted or unsubstituted 5- or 6-membered heterocyclic ring having at least one nitrogen heteroatom, boronic acid, or

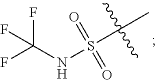 [SMILES: *S(=O)(=O)NC(F)(F)F]

and

n is 1 or 2.

In various embodiments, at least one of R¹ or R² is a substituted or unsubstituted linear or branched alkyl having at least 2 carbon atoms. In further embodiments, R¹ is hydrogen or a C1 to C6 alkyl. For example, in some embodiments, R¹ is butyl. In various embodiments, R² is cycloalkyl (e.g., cyclopentyl).

In various embodiments, R¹ and R² are selected from the group consisting of:

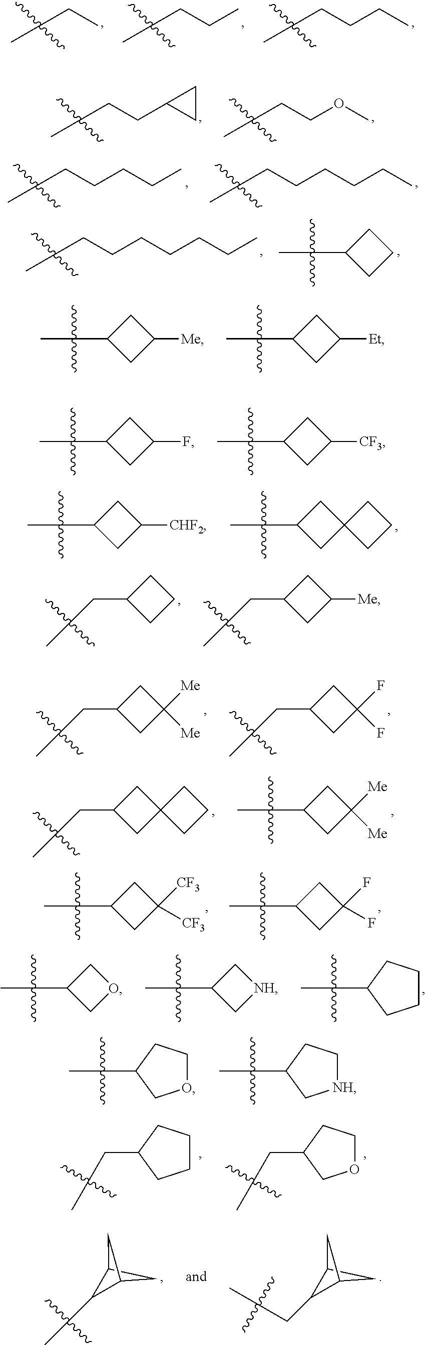

In various embodiments, R³ is —Y—C(O)R⁴. In some embodiments, R³ is —Z—N(R⁵)(R⁶). In further embodiments, R³ is —Z-A.

As noted above, A can be a substituted or unsubstituted 5- or 6-membered heterocyclic ring having at least one nitrogen heteroatom. In some embodiments, A is a substituted or unsubstituted 5- or 6-membered heterocyclic ring having at least two, three, or four nitrogen heteroatoms. In some embodiments, A is a substituted or unsubstituted 5- or 6-membered heterocyclic ring having at least one nitrogen heteroatom and at least one other heteroatom selected from oxygen or sulfur. In various embodiments, A can be boronic acid or

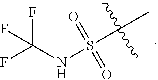 [SMILES: *S(=O)(=O)NC(F)(F)F]

In various embodiments, A is:

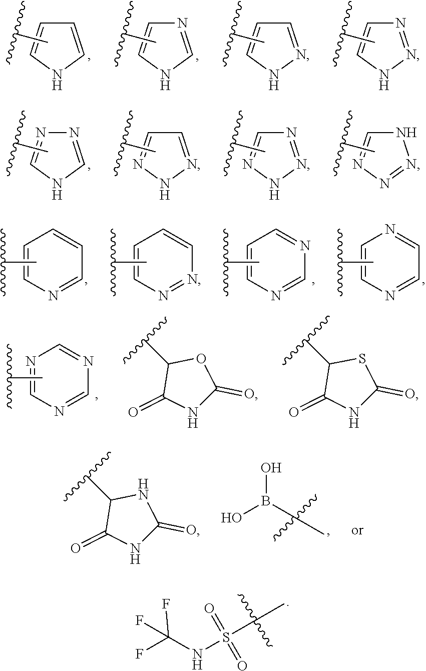

In certain embodiments, A is

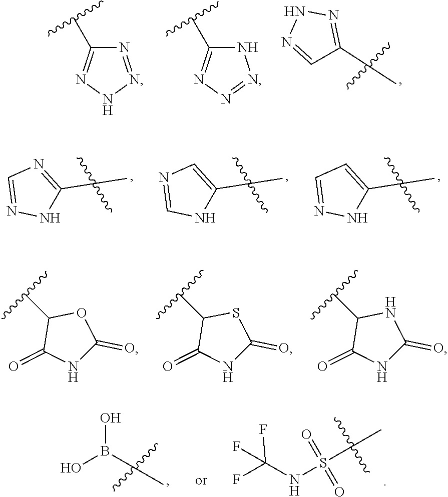

In certain embodiments, R³ is selected from the group consisting of:

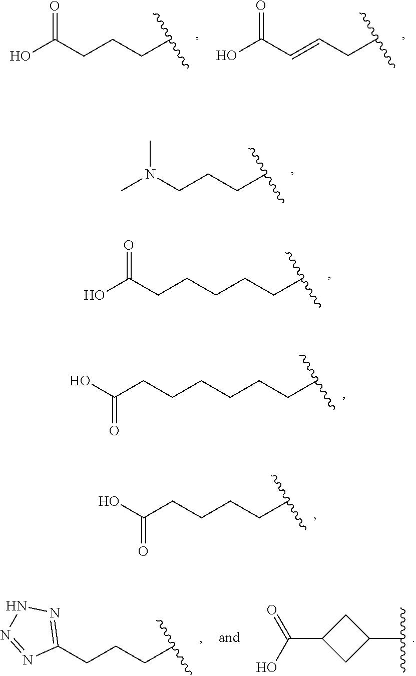

In various embodiments, R⁴ is —OW or —N(R⁸)(R⁹).

In various embodiments, X¹ and X² are each independently hydrogen, substituted or unsubstituted C1 to C6 alkyl or halo. In some embodiments, X¹ and X² are each independently C1 to C6 alkyl, fluoro, chloro, bromo, or iodo. In certain embodiments, X¹ and X² are each independently methyl, fluoro, or chloro.

In various embodiments, R⁵, R⁶, R⁷, R⁸, R⁹, R¹⁰, R¹¹ and R¹² are each independently hydrogen or alkyl. For example, in some embodiments, R⁵, R⁶, R⁷, R⁸, R⁹, R¹⁰, R¹¹, and R¹² are each independently hydrogen or a C1 to C3 alkyl.

In various embodiments, Y and Z are each independently substituted or unsubstituted alkylene having 2 to 10 carbons, substituted or unsubstituted alkenylene having from 2 to 10 carbons, or substituted or unsubstituted arylene. In some embodiments, Y and Z are each independently alkylene having 2 to 10 carbons, alkenylene having from 2 to 10 carbons, or phenylene. Y and Z can also each independently be cycloalkylene having 4 to 10 carbons. In certain embodiments, Y is an alkylene or an alkenylene having 3 to 8 carbons or 3 to 7 carbons. For example, Y can be an alkylene or any alkenylene having 4 carbons. In further embodiments, Z is an alkylene having 2 to 4 carbons. For example, Z can be an alkylene having 3 or 4 carbons.

In various embodiments, Y or Z can be selected from the group consisting of

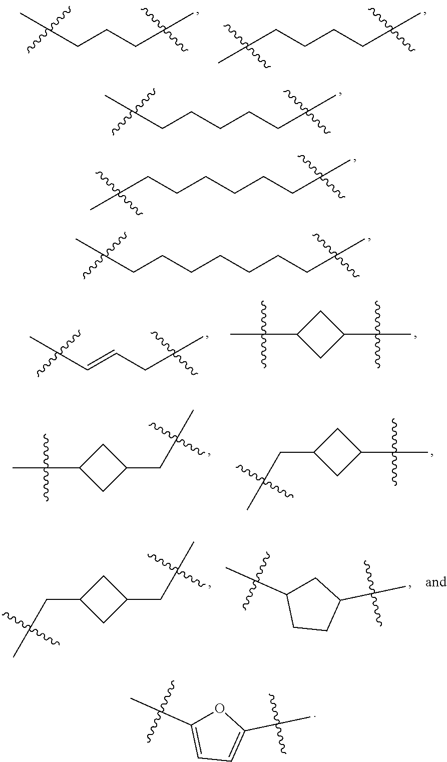

In various embodiments, when Y is an alkylene having 2 to 3 carbons then both X¹ and X² are each fluoro or each substituted or unsubstituted alkyl (e.g., methyl or ethyl). In some embodiments, Y is not an alkylene having 3 carbons. In certain embodiments, R⁷ is not hydrogen or a C1 to C6 alkyl. In some embodiments, X¹ and/or X² are not halo. In certain embodiments, X¹ and/or X² are not chloro. In some embodiments, R¹ and/or R² are not alkyl.

In accordance with the embodiments described herein, the compound of Formula (I) may be selected from the group consisting of:

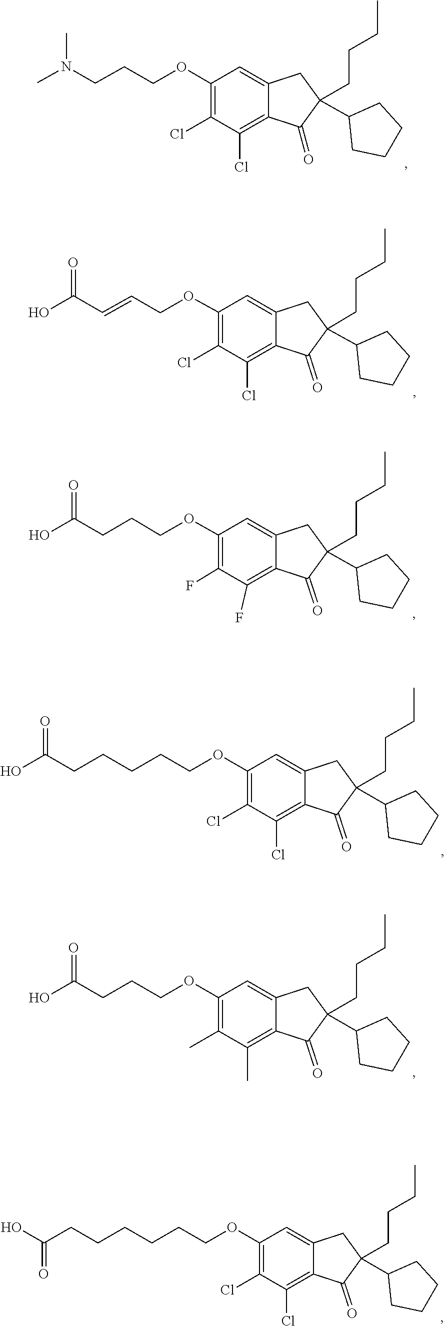 [SMILES: CCCCC1(C2CCCC2)Cc2cc(OCCCN(C)C)c(Cl)c(Cl)c2C1=O ; CCCCC1(C2CCCC2)Cc2cc(OC/C=C/C(=O)O)c(Cl)c(Cl)c2C1=O ; CCCCC1(C2CCCC2)Cc2cc(OCCCC(=O)O)c(F)c(F)c2C1=O ; CCCCC1(C2CCCC2)Cc2cc(OCCCCCC(=O)O)c(Cl)c(Cl)c2C1=O ; CCCCC1(C2CCCC2)Cc2cc(OCCCC(=O)O)c(C)c(C)c2C1=O ; CCCCC1(C2CCCC2)Cc2cc(OCCCCCCC(=O)O)c(Cl)c(Cl)c2C1=O]

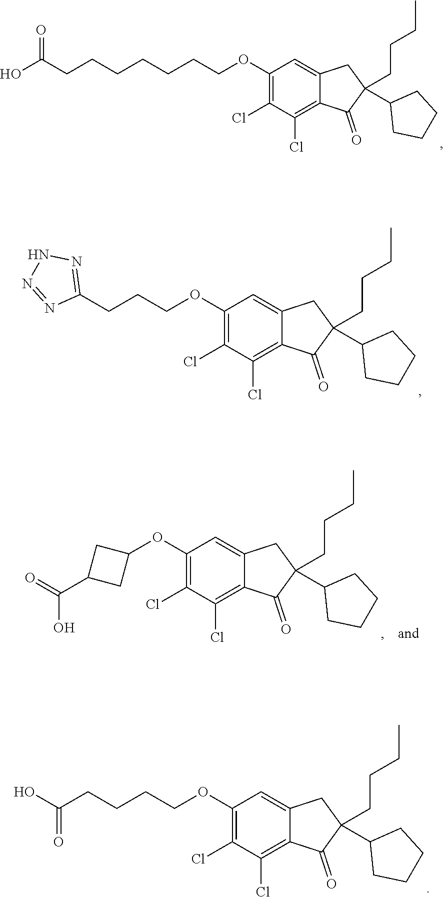 [SMILES: CCCCC1(C2CCCC2)Cc2cc(OCCCCCCCC(=O)O)c(Cl)c(Cl)c2C1=O ; CCCCC1(C2CCCC2)Cc2cc(OCCCc3nn[nH]n3)c(Cl)c(Cl)c2C1=O ; CCCCC1(C2CCCC2)Cc2cc(OC3CC(C(=O)O)C3)c(Cl)c(Cl)c2C1=O ; CCCCC1(C2CCCC2)Cc2cc(OCCCCC(=O)O)c(Cl)c(Cl)c2C1=O]

Various compounds of Formula (I) advantageously can modulate or inhibit a SWELL1 channel. In certain embodiments, the compound of Formula (I) has a higher potency at modulating or inhibiting a SWELL1 channel than an equivalent amount of DCPIB (4-[2[butyl-6,7-dichloro-2-cyclopentyl-2,3-dihydro-1-oxo-1H-inden-5-yl)oxy]butanoic acid). Therefore, they can be used to treat conditions and diseases associated with impaired SWELL1 activity.

Various aspects of the invention include methods for increasing insulin sensitivity and/or treating obesity, diabetes (e.g., Type I or Type II diabetes), nonalcoholic fatty liver disease, a metabolic disease, hypertension, stroke, vascular tone, and systemic arterial and/or pulmonary arterial blood pressure and/or blood flow in a subject in need thereof. Various aspects of the invention also include methods for treating an immune deficiency or infertility caused by insufficient or inappropriate SWELL1 activity in a subject in need thereof. In various aspects, the immune deficiency can include agammaglobulinemia. In further aspects, the infertility can be a male infertility caused by, for example, abnormal sperm development due to insufficient or inappropriate SWELL1 activity. Various aspects of the invention also include methods for treating or restoring exercise capacity and/or improving muscle endurance. In further aspects, methods are provided for treating a muscular disorder in a subject need thereof. The muscular disorder can include skeletal muscle atrophy. As the SWELL1-LRRC8 complex also regulates myogenesis, methods are also provide for regulating myogenic differentiation and insulin-P13K-AKT-AS160, ERK1/2 and mTOR signaling in myotubules. In general, these methods comprise administering to the subject a therapeutically effective amount of the compound of Formula (I).

In the various methods described herein, the administration of the compound is sufficient to upregulate the expression of SWELL1 or alter expression of a SWELL1-associated protein. In some embodiments, the administration of the compound is sufficient to stabilize SWELL1-LRRC8 channel complexes or a SWELL1-associated protein. In further embodiments, the administration of the compound is sufficient to promote membrane trafficking and activity of SWELL1-LRRC8 channel complexes or a SWELL1-associated protein. In some embodiments, the SWELL1-associated protein is selected from the group consisting of LRRC8, GRB2, Cav1, IRS1, or IRS2. In various methods described herein, the administration of the compound is sufficient to augment SWELL1 mediated signaling.

In accordance with the various methods of the present invention, a pharmaceutical composition comprising a compound of Formula (I) is administered to the subject in need thereof. The pharmaceutical composition can be administered by a routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. In various embodiments, administration is selected from the group consisting of oral, intranasal, intraperitoneal, intravenous, intramuscular, rectal, and transdermal.

The determination of a therapeutically effective dose for any one or more of the compounds described herein is within the capability of those skilled in the art. A therapeutically effective dose refers to that amount of active ingredient which provides the desired result. The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Typically, the normal dosage amount of the compound can vary from about 0.05 to about 100 mg per kg body weight depending upon the route of administration. Guidance as to particular dosages and methods of delivery is provided in the literature and generally available to practitioners in the art. It will generally be administered so that a daily oral dose in the range, for example, from about 0.1 mg to about 75 mg, from about 0.5 mg to about 50 mg, or from about 1 mg to about 25 mg per kg body weight is given. The active ingredient can be administered in a single dose per day, or alternatively, in divided doses (e.g., twice per day, three time a day, four times a day, etc.). In general, lower doses can be administered when a parenteral route is employed. Thus, for example, for intravenous administration, a dose in the range, for example, from about 0.05 mg to about 30 mg, from about 0.1 mg to about 25 mg, or from about 0.1 mg to about 20 mg per kg body weight can be used.

A pharmaceutical composition for oral administration can be formulated using pharmaceutically acceptable carriers known in the art in dosages suitable for oral administration. Such carriers enable the pharmaceutical compositions to be formulated as tablets, pills, dragees, capsules, liquids, gels, syrups, slurries, suspensions, and the like, for ingestion by the subject. In certain embodiments, the composition is formulated for parenteral administration. Further details on techniques for formulation and administration can be found in the latest edition of REMINGTON'S PHARMACEUTICAL SCIENCES (Mack Publishing Co., Easton, Pa., which is incorporated herein by reference). After pharmaceutical compositions have been prepared, they can be placed in an appropriate container and labeled for treatment of an indicated condition. Such labeling would include amount, frequency, and method of administration.

In addition to the active ingredients (e.g., the compound of Formula (I)), the pharmaceutical composition can contain suitable pharmaceutically acceptable carriers comprising excipients and auxiliaries that facilitate processing of the active compounds into preparations which can be used pharmaceutically. As used herein, the term “pharmaceutically acceptable carrier” means a non-toxic, inert solid, semi-solid or liquid filler, diluent, encapsulating material, or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil; and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; artificial cerebral spinal fluid (CSF), and phosphate buffer solutions, as well as other non-toxic compatible lubricants such as sodium lauryl sulfate and magnesium stearate, as well as coloring agents, releasing agents, coating agents, sweetening, flavoring, and perfuming agents, preservatives and antioxidants can also be present in the composition, according to the judgment of the formulator based on the desired route of administration.

Unless otherwise indicated, the alkyl, alkenyl, and alkynyl groups described herein preferably contains from 1 to 20 carbon atoms in the principal chain. They may be straight or branched chain or cyclic (e.g., cycloalkyls). Alkenyl groups can contain saturated or unsaturated carbon chains so long as at least one carbon-carbon double bond is present. Alkynyl groups can contain saturated or unsaturated carbon chains so long as at least one carbon-carbon triple bond is present. Unless otherwise indicated, the alkoxy groups described herein contain saturated or unsaturated, branched or unbranched carbon chains having from 1 to 20 carbon atoms in the principal chain.

Unless otherwise indicated herein, the term “aryl” refers to monocyclic, bicyclic or tricyclic aromatic groups containing from 6 to 14 ring carbon atoms and including, for example, phenyl. The term “heteroaryl” refers to monocyclic, bicyclic or tricyclic aromatic groups having 5 to 14 ring atoms and containing carbon atoms and at least 1, 2 or 3 oxygen, nitrogen or sulfur heteroatoms.

Having described the invention in detail, it will be apparent that modifications and variations are possible without departing from the scope of the invention defined in the appended claims.

### EXAMPLES

The following non-limiting examples are provided to further illustrate the present invention.

### Example 1: Synthesis and Screening of Compounds Having Improved Affinity for SWELL1

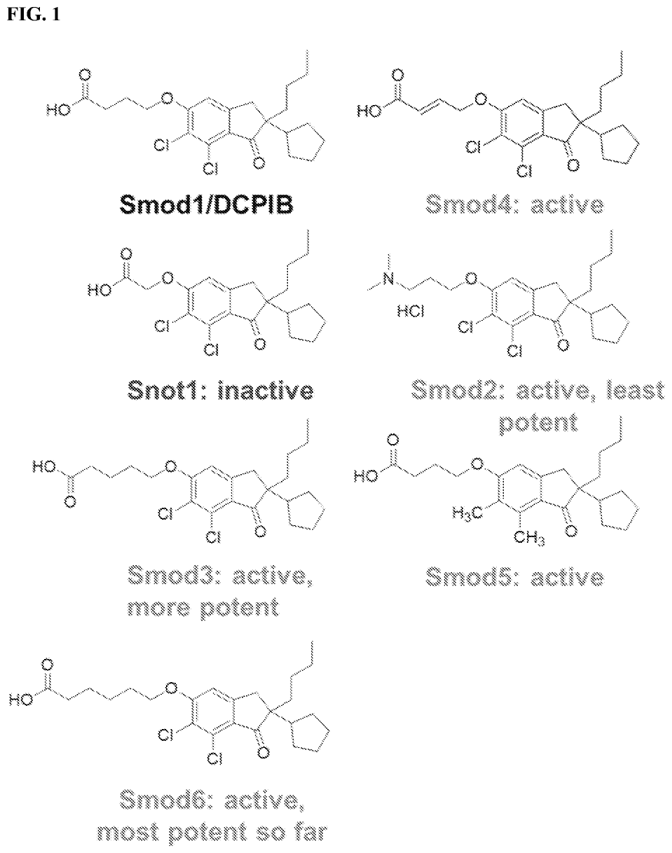

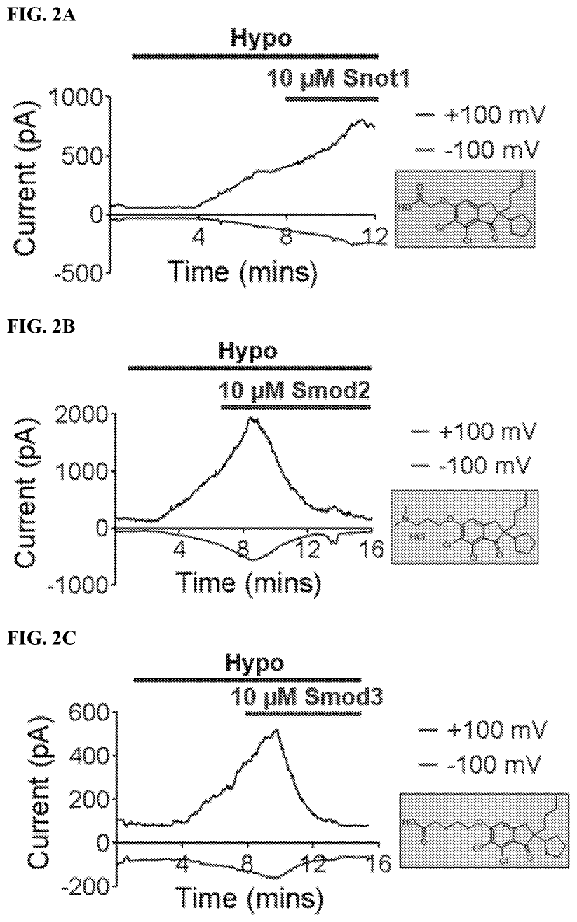

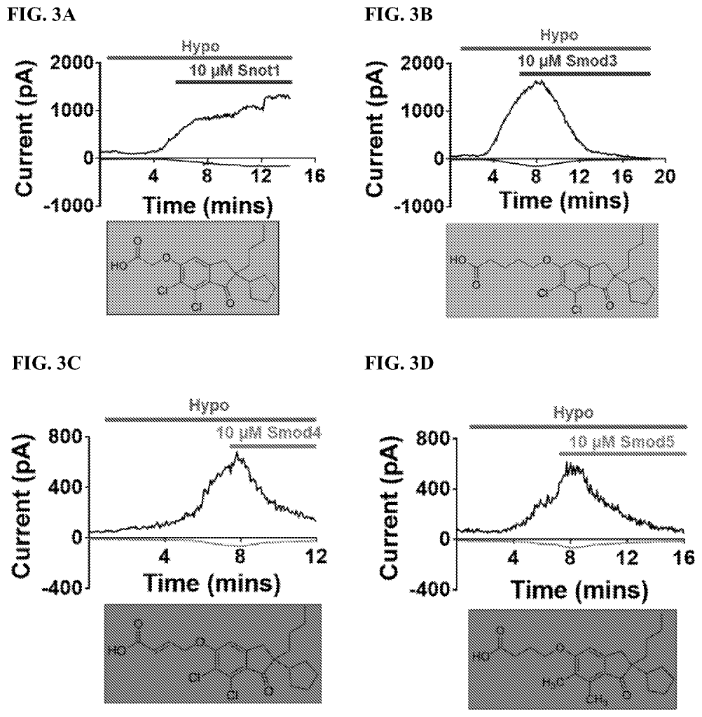

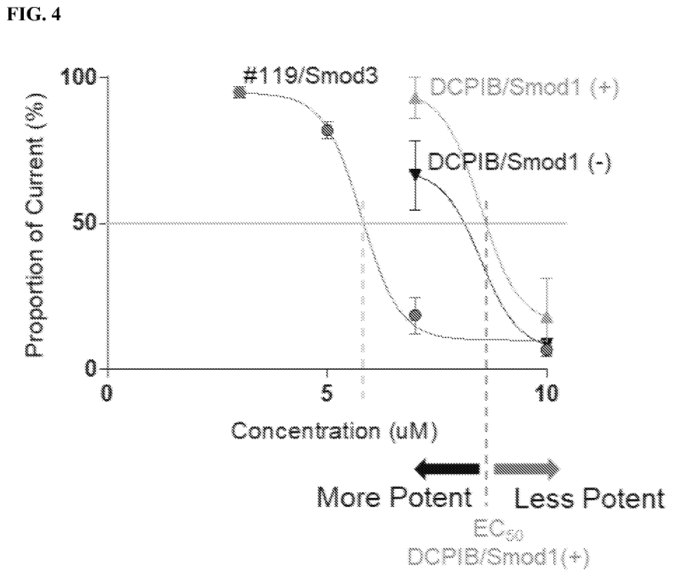

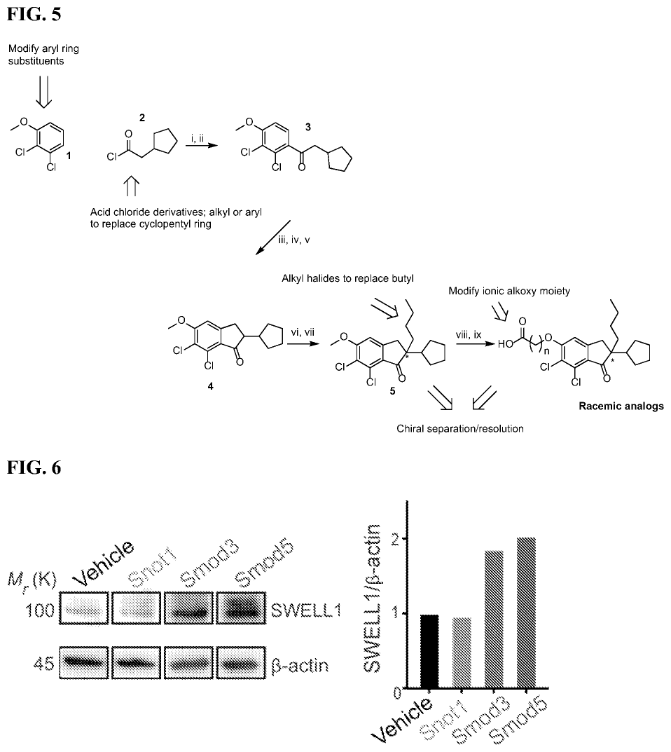

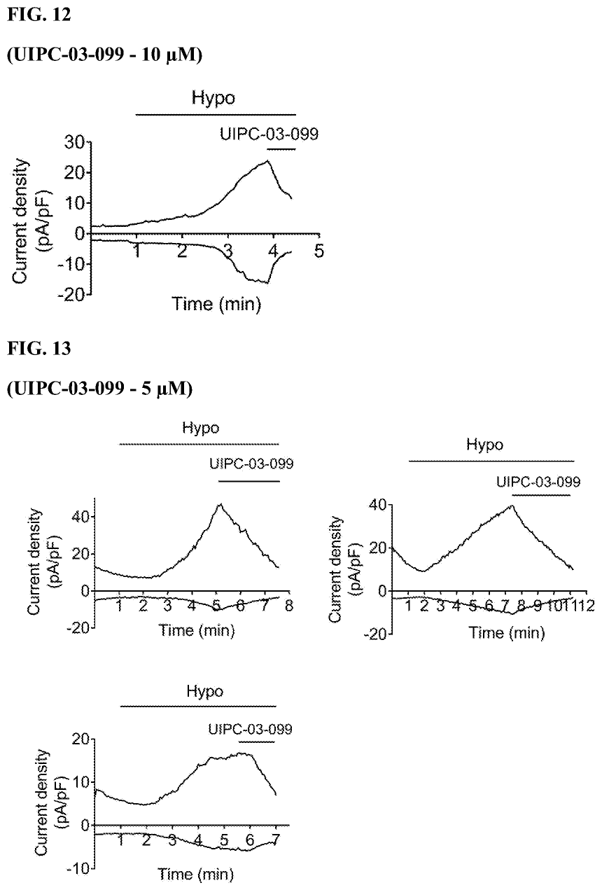

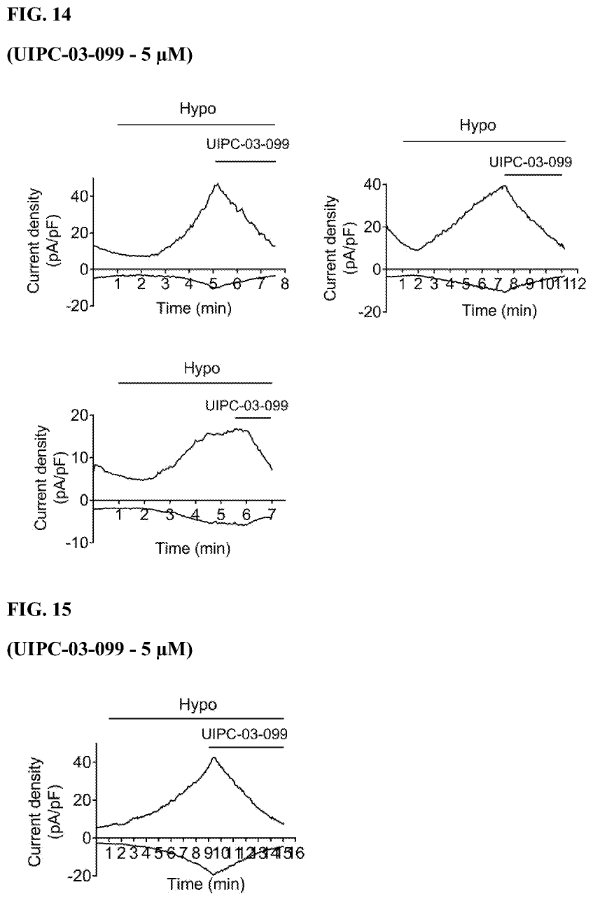

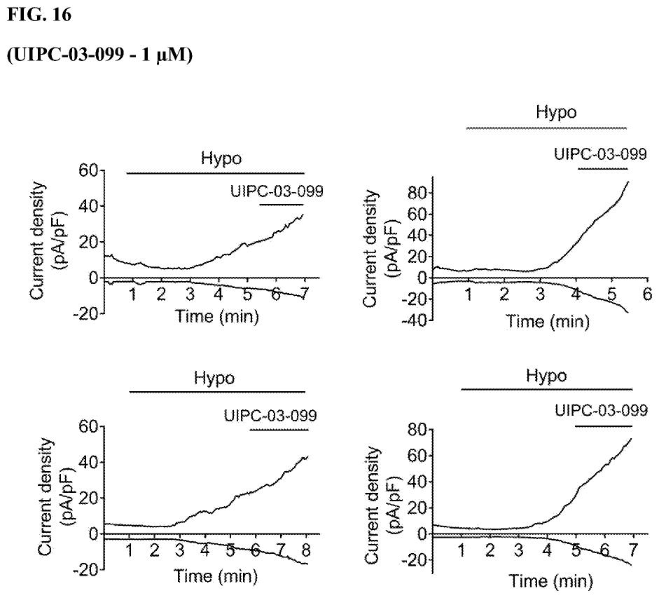

A series of compounds (Smod compounds) were synthesized to evaluate the role of a butyrate side chain and aryl substituents on activity (see FIG. 1 and Table 1 below). In preliminary patch-clamp experiments to screen for compounds that preserve or enhance SWELL1 modulatory activity, unique structural derivatives were identified with I_{Cl,SWELL} inhibitory activity (Smod 2-6, FIGS. 2, 3, and 12-16, as well as Table 1 below). Notably, the aminopropyl group afforded active Smod2. In vitro channel inhibitory activity was also maintained with Smod3-5 (FIG. 3). Note that compounds were also identified that lack activity, and therefore are not SWELL1 modulators (i.e., Snot1, FIGS. 2A and 3A). FIG. 4 summarizes three dose response curves of isolated enantiomers of Smod1 (+ and −) compared to Smod3. Smod3 shows a strong shift in the EC₅₀ demonstrating its higher potency. FIG. 5 summarizes the synthetic scheme used to generate these compounds.

TABLE 1

| | | | Activity | | |
|---|---|---|---|---|---|
| Sr. | Compound | | | | Not |
| No. | ID: | Structure | Active | Inactive | evaluated |
| 1* 2* | UICK-IV- 101a (+) (Snot1) UICK-IV- 101b (−) | 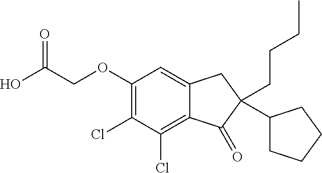 [SMILES: CCCCC1(C2CCCC2)Cc2cc(OCC(=O)O)c(Cl)c(Cl)c2C1=O] | | X X | |
| 3 | UICK-IV- 105a (±) | 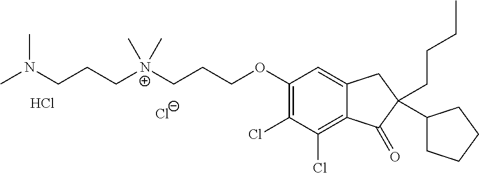 [SMILES: CCCCC1(C2CCCC2)Cc2cc(OCCC[N+](C)(C)CCCN(C)C)c(Cl)c(Cl)c2C1=O ; Cl ; [Cl-]] | | X | |
| 4 | UICK-IV- 105b (±) (Smod-2) | 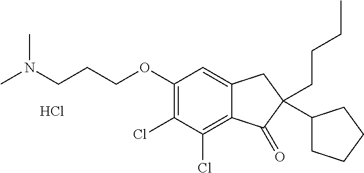 [SMILES: CCCCC1(C2CCCC2)Cc2cc(OCCCN(C)C)c(Cl)c(Cl)c2C1=O ; Cl] | X | | |
| 5 | UICK-IV-119 (±) (Smod-3) | 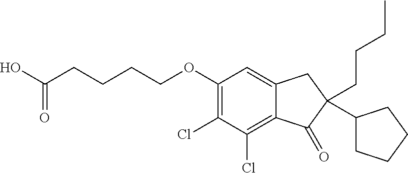 [SMILES: CCCCC1(C2CCCC2)Cc2cc(OCCCCC(=O)O)c(Cl)c(Cl)c2C1=O] | X | | |
| 6* 7* | UICK-IV-117 (+) UICK-IV-125 (−) | 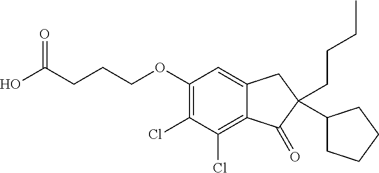 [SMILES: CCCCC1(C2CCCC2)Cc2cc(OCCCC(=O)O)c(Cl)c(Cl)c2C1=O] | X X | | |
| 8 | UIPC-II-172 (±) | 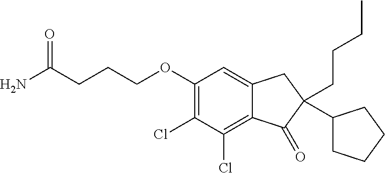 [SMILES: CCCCC1(C2CCCC2)Cc2cc(OCCCC(N)=O)c(Cl)c(Cl)c2C1=O] | | X | |
| 9 | UIPC-II-173 (±) | 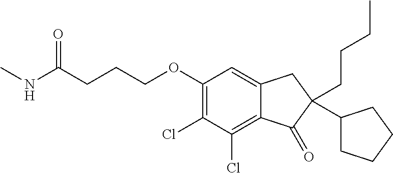 [SMILES: CCCCC1(C2CCCC2)Cc2cc(OCCCC(=O)NC)c(Cl)c(Cl)c2C1=O] | | X | |
| 10 | UIPC-II-179 (±) (Smod-4) | 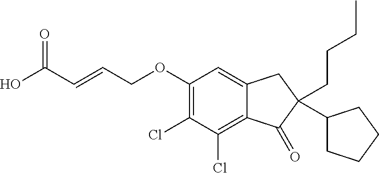 [SMILES: CCCCC1(C2CCCC2)Cc2cc(OC/C=C/C(=O)O)c(Cl)c(Cl)c2C1=O] | X | | |
| 11 | UIPC-II-183 (±) | 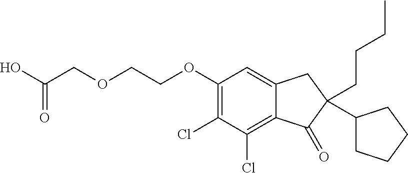 [SMILES: CCCCC1(C2CCCC2)Cc2cc(OCCOCC(=O)O)c(Cl)c(Cl)c2C1=O] | | X | |
| 12 | UIPC-II-187B (±) (Smod-5) | 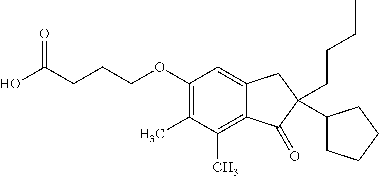 [SMILES: CCCCC1(C2CCCC2)Cc2cc(OCCCC(=O)O)c(C)c(C)c2C1=O] | | X | |
| 13 | UIPC-III- 045B (±) Smod-6 | 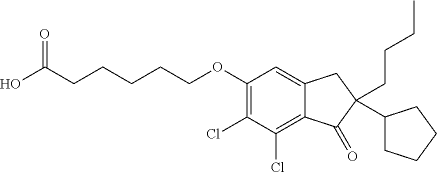 [SMILES: CCCCC1(C2CCCC2)Cc2cc(OCCCCCC(=O)O)c(Cl)c(Cl)c2C1=O] | X | | |
| 14 | UIPC-III- 063B (±) Smod7 | 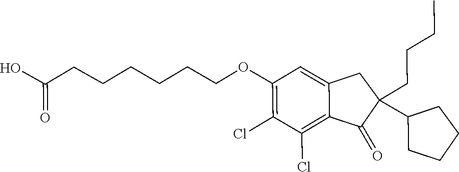 [SMILES: CCCCC1(C2CCCC2)Cc2cc(OCCCCCCC(=O)O)c(Cl)c(Cl)c2C1=O] | X | | |
| 15 | UIPC-III-126 | 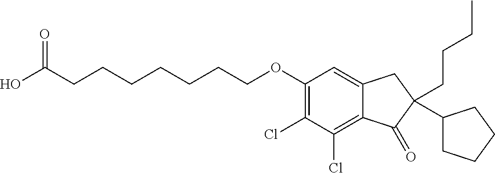 [SMILES: CCCCC1(C2CCCC2)Cc2cc(OCCCCCCCC(=O)O)c(Cl)c(Cl)c2C1=O] | | | |
| 16 | UIPC-III- 124B | 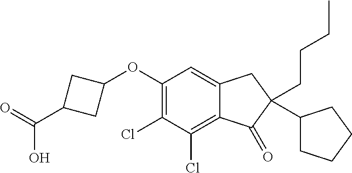 [SMILES: CCCCC1(C2CCCC2)Cc2cc(OC3CC(C(=O)O)C3)c(Cl)c(Cl)c2C1=O] | | | |
| 17 | UIPC-03-099 | 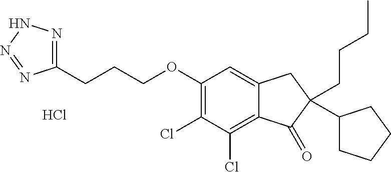 [SMILES: CCCCC1(C2CCCC2)Cc2cc(OCCCc3nn[nH]n3)c(Cl)c(Cl)c2C1=O ; Cl] | X | | |
| 18 | UIPC-III- 083B Snot2 SN-072 | 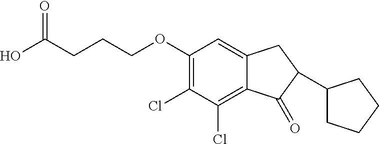 [SMILES: O=C(O)CCCOc1cc2c(c(Cl)c1Cl)C(=O)C(C1CCCC1)C2] | | X | |
| 19 | UIPC-III-092 | 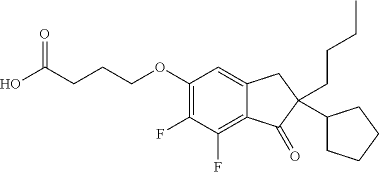 [SMILES: CCCCC1(C2CCCC2)Cc2cc(OCCCC(=O)O)c(F)c(F)c2C1=O] | X | | |

*Activity was tested on individual isomers (e.g., + or −, as indicated).

### Example 2: Effect of Compounds on SWELL1 Protein Expression and Glucose Metabolism In Vivo

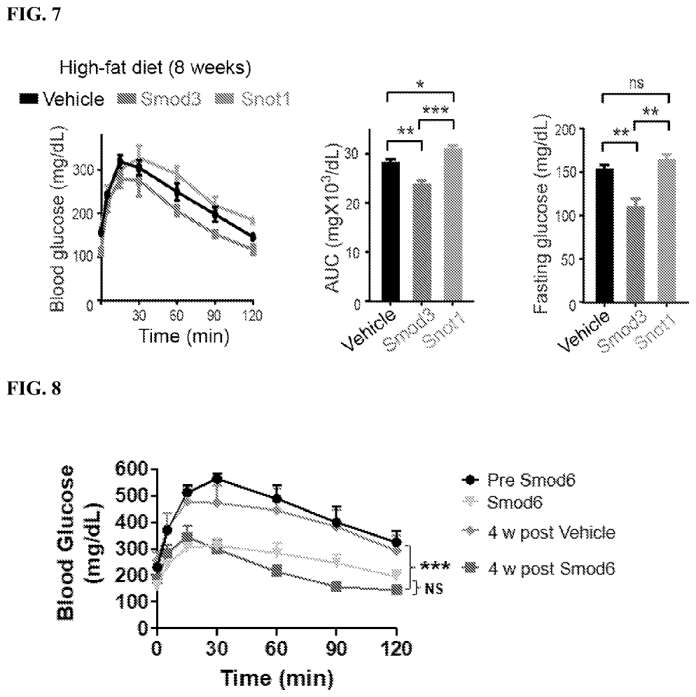

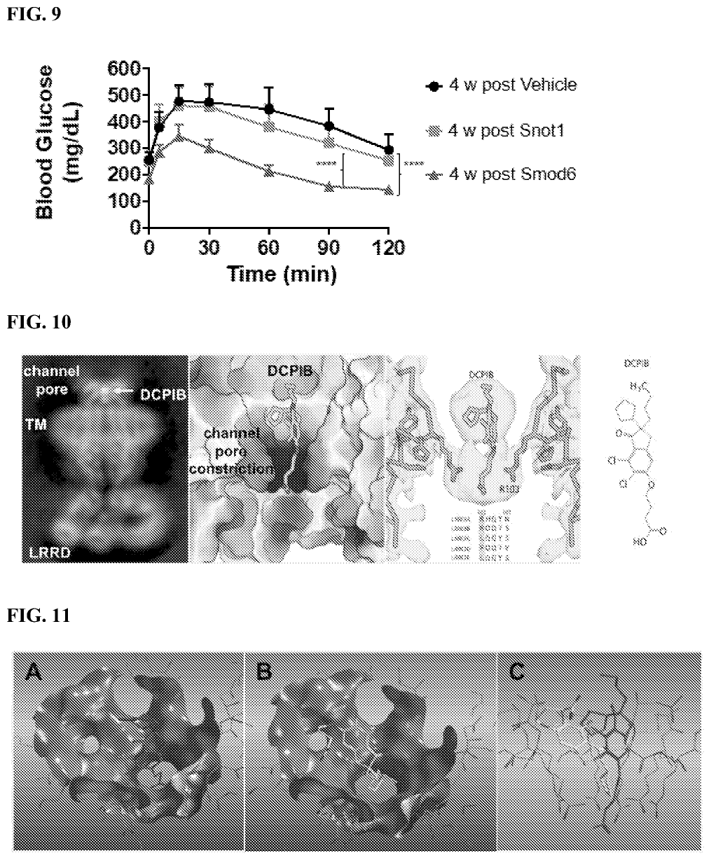

SWELL1 expression in vivo by channel-inactive Snot1 was compared to channel-active Smod3 and Smod5. Both Smod3 and Smod5 induce SWELL1 protein in 3T3-F442A adipocytes compared to vehicle, while Snot1 is ineffective (FIG. 6). Moreover, Smod3, and not Snot1 (5 mg/kg i.p.×4 days) improve glucose tolerance (GTT, Area under the curve) and fasting glucose (FG) in mice raised on HFD for 8 weeks in pilot studies (FIG. 7). Similarly, SWELL1 channel active Smod6 and not SWELL1 channel inactive Snot1, nor vehicle sustain improved glucose tolerance in T2D HFD fed mice 4 weeks after discontinuing treatment in T2D HFD fed mice after 20 weeks HFD (FIGS. 8 and 9).

### Example 3: Structure-Function Investigation into Smod Compounds and their Interaction with SWELL Channel

The recent Cryo-EM structure of Smod1 bound with a SWELL1 homohexamer22 was used to generate binding models in an effort to explain activity profiles for the Smod compounds described in Example 1. As shown in FIG. 10 and FIGS. 11A and 11C the butyrate chain of Smod1 protrudes through the neck of the SWELL1 channel and interacts with R103 residue(s). The remainder of Smod1 structure occupies hydrophobic binding space along the arginine side chains and immediately above the channel neck. This binding mode, and similar docking of the Smods and Snots evaluated in preliminary work, explains 1) the role of butyrate chain, and length of the chain, for SWELL1 binding (i.e., Snot1 versus Smod1, 3 and 4), 2) the requirement of carboxylate for activity (amides in place of Smod1 carboxylate group affords inactive Smods), and 3) that changing the aryl chlorines to aryl methyl groups (Smod5) did not significantly alter activity. This binding mode might appear inconsistent with the cationic Smod2 regulating SWELL1 activity because the tertiary amine would not likely interact with R103 residues. However, one explanation for Smod2 activity is that the SWELL1-LRRC8 channel complex is not homo-hexamer of SWELL1 in nature (FIG. 10), and a pattern of F103 with L103 replacing some R103 subunits (i.e., a SWELL1-LRRC8c/d/e hetero-hexamer) could create the environment for a cation-Pi interaction. A second possible explanation for Smod2 binding SWELL1 was revealed through modeling studies, where in silico docking showed the Smods to be flipped 180 degrees in preferred docking poses (FIG. 11B). In this alternative binding mode, hydrophobic binding interactions are maintained above the neck of the channel, while terminal cationic or anionic groups on the alkoxy chain interact with amino acid side chains or backbone amides of the channel wall. Combined, these results show that different Smods might bind in different orientations within different SWELL1 channels. As such, differences in LRRC8 subunit composition in different tissues (differences at position 103 for different hetero-hexamers as well as amino acid variations above the channel neck) can afford the possibility to identify Smod compounds that display tissue-selective inhibition of SWELL1-LRRC8 channel complexes. Indeed, given the broad tissue expression of SWELL1-LRRC8 channel complexes, the ability to selectively modulate specific SWELL1-LRRC8 stoichiometries in different tissues or cell-types may become very important.

### Example 4: Materials and Methods for Examples 6 to 12

### Patients

Human islets and adipocytes were obtained and cultured as described previously (Kang et al., 2018; Zhang et al., 2017). The patients involved in the study were anonymous and information such as gender, age, HbA1c, glucose levels and BMI only were available to the research team.

### Animals

All C57BL/6 mice involved in study were purchased from Charles River Labs. Both KK.Cg-Ay/J (KKN) and KK.Cg-Aa/J (KKAa) mice involved in study were gender and age-matched mice obtained from Jackson Labs (Stock No: 002468) and bred up for experiments. The mice were fed ad libitum with either regular chow (RC) or high-fat diet (Research Diets, Inc., 60 kcal % fat) with free access to water and housed in a light-, temperature- and humidity-controlled room. For high-fat diet (HFD) studies, only male mice were used and were started on HFD regimen at the age of 6-9 weeks. For all experiments involving KKN and KKAa mice, both males and females were used at approximately 50/50 ratio. In all experiments involving mice, investigators were kept blinded both during the experiments and subsequent analysis.

### 3T3-F442A Cell Line

3T3-F442A (Sigma-Aldrich) cells were maintained in 90% DMEM (25 mM D-Glucose and 4 mM L-Glutamine) containing 10% fetal bovine serum (FBS) and 100 IU penicillin and 100 μg/ml streptomycin.

### HEK-293 Cell Line

HEK-293 (ATCC® CRL-1573™) cells were maintained in 90% DMEM (25 mM D-Glucose and 4 mM L-Glutamine) containing 10% fetal bovine serum (FBS) and 100 IU penicillin and 100 μg/ml streptomycin. Overexpression of plasmid DNA in HEK-293 cells were carried out using Lipofectamine 2000 (Invitrogen) reagent.

### Small Molecule Treatment

All compounds were dissolved in Kolliphor® EL (Sigma, #C5135). Either vehicle (Kolliphor® EL), SN-401 (DCPIB, 5 mg/kg of body weight/day, Tocris, D1540), SN-403, SN-406, SN-407 or SN071 were administered i.p. as indicated using 1 cc syringe/26G×½ inch needle daily for 4-10 days, and in one experiment, SN-401 was administered daily for 8 weeks. SN-401, formulated as above, was also administered by oral gavage at 5 mg/kg/day for 5 days using a 20G×1.5 inch reusable metal gavage needle.

### Adenovirus

Adenovirus type 5 with Ad5-RIP2-GFP (4.1×10¹⁰ PFU/ml) and Ad5-CAG-LoxP-stop-LoxP-3×Flag-SWELL 1 (1×10¹⁰ PFU/ml) were obtained from Vector Biolabs. Adenovirus type 5 with Ad5-CMV-Cre-wt-1RES-eGFP (8×10¹⁰ PFU/ml) was obtained from the University of Iowa Viral Vector Core.

### Cell Culture

Wildtype (WT) and SWELL1 knockout (KO) 3T3-F442A (Sigma-Aldrich) cells were cultured and differentiated as described previously (Zhang et al., 2017). Preadipocytes were maintained in 90% DMEM (25 mM D-Glucose and 4 mM L-Glutamine) containing 10% fetal bovine serum (FBS) and 100 IU penicillin and 100 μg/ml streptomycin on collagen-coated (rat tail type-I collagen, Corning) plates. Upon reaching confluency, the cells were differentiated in the above-mentioned media supplemented with 5 μg/ml insulin (Cell Applications) and replenished every other day with the differentiation media. For insulin signaling studies on WT and KO adipocytes with or without SWELL1 overexpression (O/E), the cells were differentiated for 10 days and transduced with Ad5-CAG-LoxP-stop-LoxP-SWELL1-3×Flag virus (MOI 12) on day 11 in 2% FBS containing differentiation medium. To induce the overexpression, Ad5-CMV-Cre-wt-lRES-eGFP (MOI 12) was added on day 13 in 2% FBS containing differentiation medium. The cells were then switched to 10% FBS containing differentiation medium from day 15 to 17. On day 18, the cells were starved in serum free media for 6 h and stimulated with 0 and 10 nM insulin for either 5 or 15 min Either Ad5-CAG-LoxP-stop-LoxP-SWELL1-3×Flag or Ad5-CMV-Cre-wt-1RES-eGFP virus transduced cells alone were used as controls. Based on GFP fluorescence, viral transduction efficiency was ˜90%.

For SN-401 treatment and insulin signaling studies in 3T3-F442A preadipocytes, the cells were incubated with either vehicle (DMSO) or 10 μM SN-401 for 96 h. The cells were serum starved for 6 h (+DMSO or SN-401) and washed with PBS three times and stimulated with 0, 3 and 10 nM insulin containing media for 15 mins prior to collecting lysates. In the case of 3T3-F442A adipocytes, the WT and KO cells were treated with either vehicle (DMSO), 1 or 10 μM SN-40X following 7-11 days of differentiation for 96 hand then stimulated with 0 and 10 nM insulin/serum containing media (+DMSO or SN-40X) for 15-30 min for SWELL1 detection. For AKT and AS160 signaling, the serum starved cells in the presence of compounds were washed twice in hypotonic buffer (240 mOsm) and then incubated at 37° C. in hypotonic buffer for 10 min followed by stimulation with insulin/serum containing media. To simulate gluco-lipotoxicity, sodium palmitate was dissolved in 18.4% fatty-acid free BSA at 37° C. in DMEM medium with 25 mM glucose to obtain a conjugation ratio of 1:3 palmitate:BSA (Busch et al., 2002). As described above, the 3T3-F442A adipocytes were incubated with vehicle or SN-401, SN-406, SN072 at 10 μM for 96 h and treated with 1 mM palmitate for additional 16 hand lysates were collected and further processed.

### Molecular Docking

SN-401 and its analogs were docked into the expanded state structure of a LRRCBA-SN-401 homo-hexamer in MSP1E3D1 nanodisc (PDB ID: 6NZZ) using Molecular Operating Environment (MOE) 2016.08 software package [Chemical Computing Group (Montreal, Canada)]. The 3D structure obtained from PDB (PDB ID: 6NZZ) was prepared for docking by first generating the missing loops using the loop generation functionality in Yasara software package followed by sequentially adding hydrogens, adjusting the 3D protonation state and performing energy minimization using Amber10 force-field in MOE. The ligand structures to be docked were prepared by adjusting partial charges followed by energy minimization using Amber10 force-field. The site for docking was defined by selecting the protein residues within 5A from co-crystallized ligand (SN-401). Docking parameters were set as Placement: Triangle matcher; Scoring function: London dG; Retain Poses: 30; Refinement: Rigid Receptor; Re-scoring function: GBVI/WSA dG; Retain poses: 5. Binding poses for the compounds were predicted using the above validated docking algorithm.

### Electrophysiology

Patch-clamp recordings of β-cells and mature adipocytes were performed as described previously (Kang et al., 2018; Zhang et al., 2017). 3T3-F442A WT and KO preadipocytes were prepared as described in the Cell culture section above. For SWELL1 overexpression recordings, preadipocytes were first transduced with Ad5-CAG-LoxP-stop-LoxP-3×Flag-SWELL1 (MOI 12) in 2% FBS culture medium for two days and then overexpression induced by adding Ad5-CMV-Cre-wt-lRES-eGFP (MOI 10-12) in 2% FBS culture medium for two more days and changed to 10% FBS containing culture media and were selected based on GFP expression (˜2-3 days). For cell recordings, islets were transduced with Ad-RIP2-GFP and then dispersed after 48-72 hours for patch-clamp experiments. GFP+ cells marked β-cells selected for patch-clamp recordings. For measuring I_{Cl,SWELL} inhibition by SN-401 congeners after activation of I_{Cl,SWELL}, HEK-293 cells were perfused with hypotonic solution (Hypo, 210 mOsm) described below and then SN-401 congeners+ Hypo applied at 10 and 7 μM to assess for % I_{Cl,SWELL} inhibition. To assess for I_{Cl,SWELL} inhibition upon application of SN-401 congeners to the closed SWELL1-LRRC8 channel, HEK-293 cells were preincubated with vehicle (or SN-401, SN-406, SN071 and SN072) for 30 mins prior to hypotonic stimulation and then stimulated with hypotonic solution+SN-401 congeners. Recordings were measured using Axopatch 2008 amplifier paired to a Digidata 1550 digitizer using pClamp 10.4 software. The extracellular buffer composition for hypotonic stimulation contains 90 mM NaCl, 2 mM CsCl, 1 mM MgCl, 1 mM CaCb, 10 mM HEPES, 10 mM Mannitol, pH 7.4 with NaOH (210 mOsm/kg). The extracellular isotonic buffer composition is same as above except for Mannitol concentration of 110 mM (300 mOsm/kg). The composition of intracellular buffer is 120 mM L-aspartic acid, 20 mM CsCl, 1 mM MgCl, 5 mM EGTA, 10 mM HEPES, 5 mM MgATP, 120 mM CsOH, 0.1 mM GTP, pH 7.2 with CsOH. All recordings were carried out at room temperature (RT) with HEK-293 cells, β-cells and 3T3-F442A cells performed in whole-cell configuration and human adipocytes in perforated-patch configuration, as previously described (Kang et al., 2018; Zhang et al., 2017).

### Western Blot

Cells were washed twice in ice-cold phosphate buffer saline and lysed in RIPA buffer (150 mM NaCl, 20 mM HEPES, 1% NP-40, 5 mM EDTA, pH 7.4) with proteinase/phosphatase inhibitors (Roche). The cell lysate was further sonicated in 10 sec cycle intervals for 2-3 times and centrifuged at 14000 rpm for 20 min at 4° C. The supernatant was collected and further estimated for protein concentration using DC protein assay kit (Bio-Rad). Fat tissues were homogenized and suspended in RIPA buffer with inhibitors in similar fashion as described above. Protein samples were further prepared by boiling in 4× laemmli buffer. Approximately 10-20 μg of total protein was loaded in 4-15% gradient gel (Bio-Rad) for separation and protein transfer was carried out onto the PVDF membranes (Bio-Rad). Membranes were blocked in 5% BSA (or 5% milk for SWELL1) in TBST buffer (0.2 M Tris, 1.37 M NaCl, 0.2% Tween-20, pH 7.4) for 1 hand incubated with appropriate primary antibodies (5% BSA or milk) overnight at 4° C. The membranes were further washed in TBST buffer before adding secondary antibody (Bio-Rad, Goat-anti-rabbit, #170-6515) in 1% BSA (or 1% milk for SWELL1) in TBST buffer for 1 h at RT. The signals were developed by chemiluminescence (Pierce) and visualized using a Chemidoc imaging system (Biorad). The images were further analyzed for band intensities using ImageJ software. Following primary antibodies were used: anti-phospho-AKT2 (#8599s), anti-AKT2 (#3063s), anti-phospho-AS160 (#4288s), anti-AS160 (#2670s) anti-GAPDH (#D16H11) and anti-β-actin (#8457s) from Cell Signaling; Rabbit polyclonal anti-SWELL1 antibody was generated against the epitope QRTKSRIEQGIVDRSE (SEQ ID NO: 13) (Pacific Antibodies).

### Immunofluorescence

3T3-F442A preadipocytes (WT, KO) and differentiated adipocytes without or with SWELL1 overexpression (WT+SWELL1 O/E, KO+SWELL1 O/E) were prepared as described in the Cell culture section on collagen coated coverslips. In the case of SWELL1 membrane trafficking, the 3T3-F442A preadipocytes were incubated in the presence of vehicle (or SN-401, SN-406 and SN071) at either 1 or 10 μM for 48 h and further processed. The cells were fixed in ice-cold acetone for 15 min at −20° C. and then washed four times with 1×PBS and permeabilized with 0.1% Triton X-100 in 1×PBS for 5 min at RT and subsequently blocked with 5% normal goat serum for 1 h at RT. Either anti-SWELL1 (1:400) or anti-Flag (1:1500, Sigma #F3165) antibody were added to the cells and incubated overnight at 4° C. The cells were then washed three times (1×PBS) prior and post to the addition of 1:1000 Alexa Flour 488/568 secondary antibody (anti-rabbit, #A11034 or anti-mouse, #A11004) for 1 hour at RT. Cells were counterstained with nuclear TO-PRO-3 (Life Technologies, #T3605) or DAPI (Invitrogen, #D1306) staining (1 μM) for 20 min followed by three washes with 1×PBS. Coverslips were further mounted on slides with Prolong Diamond anti-fading media. All images were captured using Zeiss LSM700/LSM510 confocal microscope with 63× objective (NA 1.4). SWELL1 membrane localization was quantified by stacking all the z-images and converting it into a binary image where the cytoplasmic intensity per unit area was subtracted from the total cell intensity per unit area using ImageJ software.

### Metabolic Phenotyping

Mice were fasted for 6 h prior to glucose tolerance tests (GTT). Baseline glucose levels at 0 min timepoint (fasting glucose, FG) were measured from blood sample collected from tail snipping using glucometer (Bayer Healthcare LLC). Either 1 g or 0.75 g D-Glucose/kg body weight were injected (i.p.) for lean or HFD mice, respectively and glucose levels were measured at 7, 15, 30, 60, 90 and 120 min timepoints after injection. For insulin tolerance tests (ITTs), the mice were fasted for 4 h. Similar to GTTs, the baseline blood glucose levels were measured at 0 min timepoint and 15, 30, 60, 90 and 120 min timepoints post-injection (i.p.) of insulin (HumulinR, 1 U/kg body weight for lean mice or 1.25 U/kg body weight for HFD mice). GTTs or ITTs with vehicle (or SN-401, SN-403, SN-406, SN-407 and SN071) treated groups were performed approximately 24 hours after the last injection. For insulin secretion assay, the vehicle (or SN-401, SN-406 and SN071) treated HFD mice were fasted for 6 hand injected (i.p.) with 0.75 g D-Glucose/kg body weight and blood samples were collected at 0, 7, 15 and 30 min time points in microvette capillary tubes (SARSTEDT, #16.444) and centrifuged at 2000×g for 20 min at 4° C. The collected plasma was then measured for insulin content by using Ultra-Sensitive Mouse Insulin ELISA Kit (Crystal Chem, #90080). All mice and treatment groups were assessed blindly while performing experiments.

### Murine Islet Isolation and Perifusion Assay

For patch-clamp studies involving primary mouse cells, the mice were anesthesized by injecting Avertin (0.0125 g/ml in H₂O) followed by cervical dislocation. HFD or polygenic KKAy mice treated with either vehicle (or (or SN-401, SN-406, SN-407 and SN071) were anesthesized with 1-4% isoflurane followed by cervical dislocation. Islets were further isolated as described previously (Kang et al., 2018). The perifusion of islets were performed using a PER14-02 from Biorep Technologies. For each experiment, around 50 freshly isolated islets (all from the same isolation batch) were handpicked to match size of islets across the samples and loaded into the polycarbonate perifusion chamber between two layers of polyacrylamide-microbead slurry (Bio-Gel P-4, BioRad) by the same experienced operator. Perifusion buffer contained (in mM): 120 NaCl, 24 NaHCO₃, 4.8 KCl, 2.5 CaCl, 1.2 MgSO4, 10 HEPES, 2.8 glucose, 27.2 mannitol, 0.25% w/v bovine serum albumin, pH 7.4 with NaOH (300 mOsm/kg). Perifusion buffer kept at 37° C. was circulated at 120 μI/min After 48 min of washing with 2.8 mM glucose solution for stabilization, islets were stimulated with the following sequence: 16 min of 16.7 mM glucose, 40 min of 2.8 mM glucose, 10 min of 30 mM KCl, and 12 min of 2.8 mM glucose. Osmolarity was matched by adjusting mannitol concentration when preparing solution containing 16.7 mM glucose. Serial samples were collected either every 1 or 2 min into 96 wells kept at 4° C. Insulin concentrations were further determined using commercially available ELISA kit (Mercodia). The area under the curve (AUC) for the high-glucose induced insulin release was calculated for time points between 50 to 74/84 min. At the completion of the experiments, islets were further lysed by addition of RIPA buffer and the amount of insulin was detected by ELISA.

### Drug Pharmacokinetics

The pharmacokinetic studies of SN-401/SN-406 study were performed at Charles River Laboratory as outlined below. Male C57/BL6 mice were used in the study and assessed for a single dose (5 mg/kg) administration. The compounds were prepared in Cremaphor for i.p. and p.o dose routes and in 5% ethanol, 10% Tween-20 and water mix for i.v. route at a final concentration of 1 mg/ml. Terminal blood samples were collected via cardiac venipuncture under anesthesia at timepoints 0.08, 0.5, 2, 8 h post dose for i.v and at timepoints 0.25, 2, 8, 24 h post dose for i.p. and p.o. groups respectively with a sample size of 3 mice per timepoint. The blood samples were collected in tubes with K2 EDTA anticoagulant and further processed to collect plasma by centrifugation at 3500 rpm at 5° C. for 10 min Samples were further processed in LC/MS to determine the concentration of the compounds. Non-compartmental analysis was performed to obtain the PK parameters using the PKPlus software package (Simulation Plus). The area under the plasma concentration-time curve (AUCint) is calculated from time 0 to infinity where the Cmax is the maximal concentration achieved in plasma and t112 is the terminal elimination half-life. Oral bioavailability was calculated as AUCorailAUC1v*100.

### In Vitro and in Silico ADMET

In vitro ADMET studies were performed at Charles River Laboratory as outlined below. For the Caco-2 permeability assay, the cells were cultured (DMEM, 10% FBS, 1% L-Glutamax and 1% PenStrep) for 21 days. HBSS was used as the transport buffer and the TEER measurements were taken before the start of the assay. Compounds were added apical side to determine apical to basolateral transport (A-B) and basal side to determine basolateral to apical transport (B-A). Samples (10 μL) were collected at time 0 and 2 h and diluted (5×) with transport buffer. After the quenching reaction, the samples were further diluted in MilliQ water for bioanalysis. The TEER measurements were carried out at the end of the assay and wells with significant decrease in post-assay TEER values were not included in the data and repeated again. The analyte levels (peak area ratios) were measured on apical (A) and basolateral (B) sides at To and T2h-A-B and B-A fluxes were calculated averaging 3 individual measurements. Apparent permeability (Papp, cm/sec) was calculated as dQ (flux)/(dt*area*concentration). The efflux ratio was calculated by Papp(B-A)/Papp(B-A). For the microsomal metabolic stability assay, the microsomes were diluted in potassium phosphate buffer to maintain at a final concentration of 0.5 mg/ml in the assay procedure. The compounds were diluted 10-fold in acetonitrile and incubated with the microsomes at 37° C. with gentle shaking. Samples were collected at different timepoints and quenched. The samples were mixed by vortexing for 10 min and centrifuged at 3100 rpm for 10 min at 4° C. The supernatant was diluted in water and further analyzed in LC/MS autosampler. Half-life (T_{1/2}) was calculated by the formula 0.692/slope where slope is ln(% remaining relative to Tzero vs time). Intrinsic clearance was calculated using the (CLn1)=T112*1/initial concentration*mg prep/g liver*g liver/kg body weight. For the cytochrome P450 inhibition assay, the cofactors and substrate were mixed in Potassium phosphate buffer. A stock concentration of 10 mM compounds (in DMSO) were diluted 5-fold in acetonitrile and mixed with cofactor/substrate mixture (2×). Human liver microsomes were diluted in Potassium phosphate buffer for a final concentration (2×) of 0.2 mg/ml and the reaction was initiated by mixing the microsomes with the compound/cofactor/substrate mixture at 37° C. with gentle shaking. Samples were collected at Tₒ and T30 min timepoints and quenched. The samples were then centrifuged at 3100 rpm for 5 min at 5-10° C. and the supernatant was diluted in water and further analyzed in LC/MS autosampler. % inhibition was calculated (using peak area ratios) relative to zero inhibition (full activity) and no activity (full inhibition). In silica prediction of properties and drug likeness of SN-401 and SN-406 drugs were performed using the FAF-Drugs4 and preADMET software packages.

### Hyperinsulinemic Euglycemic Glucose Clamp

Sterile silicone catheters (Dow-Corning) were placed into the jugular vein of mice under isoflurane anesthesia. Placed catheter was flushed with 200 U/ml heparin in saline and the free end of the catheter was directed subcutaneously via a blunted 14-gauge sterile needle and connected to a small tubing device that exited through the back of the animal. Mice were allowed to recover from surgery for 3 days, then received IP injections of vehicle or SN-401 (5 mg/kg) for 4 days. Hyperinsulinemic euglycemic clamps were performed on day 8 post-surgery on unrestrained, conscious mice as described elsewhere (Ayala et al., 2011; Kim et al., 2000), with some modifications. Mice were fasted for 6 h at which time insulin and glucose infusion were initiated (time 0). At 80 min prior to time 0 basal sampling was conducted, where whole-body glucose flux was traced by infusion of 0.05 μCi/min D-[3-3H]-glucose (Perkin Elmer), after a priming 5 μCi bolus for 1 minute. After the basal period, starting at time 0 D-[3-3H]-glucose was continuously infused at the 0.2 μCi/min rate and the infusion of insulin (Humulin, Eli Lilly) was initiated with a bolus of 80 mU/kg/min then followed by continuous infusion of insulin at the dose of 8 mU/kg/min throughout the assay. Fifty percent dextrose (Hospira) was infused at a variable rates (GIR) starting at the same time as the initiation of insulin infusion to maintain euglycemia at the targeted level of 150 mg/dl (8.1 mM). Blood glucose (BG) measurements were taken every ten minutes via tail vein sampling using Contour glucometer (Bayer). After mouse reached stable BG and GIR (typically, after 75 minutes since starting the insulin infusion; for some mice, a longer time was required to achieve steady state) a single bolus of 12 μCi of [1-14C]-2-deoxy-D-glucose (Perkin Elmer) in 96 μl of saline was administered. Plasma samples (collected from centrifuged blood) for determination of tracers enrichment, glucose level and insulin concentration were obtained at times −80, −20, −10, 0, and every 10 min starting at 80 min post-insulin (5 min after [1-14C]-2-deoxy-D-glucose bolus was administered) until the conclusion of the assay at 140 min. Tissue samples were then collected from mice under isofluorane anesthesia from organs of interest (e.g., liver, heart, kidney, white adipose tissue, brown adipose tissue, gastrocnemius, soleus etc.) for determination of 1-14C1-2-deoxy-D-glucose tracer uptake. Plasma and tissue samples were processed as described previously (Ayala et al., 2011). Briefly, plasma samples were deproteinized with Ba(OH)₂ and ZnSO₄ and dried to eliminate tritiated water. The glucose turnover rate (mg/kg-min) was calculated as the rate of tracer infusion (dpm/min) divided by the corrected plasma glucose specific activity (dpm/mg) per kg body weight of the mouse. Fluctuations from steady state were accounted for by use of Steele's model. Plasma glucose was measured using Analox GMD9 system (Analox Technologies).

Tissue samples (˜30 mg each) were homogenized in 750 μl of 0.5% perchloric acid, neutralized with 10 M KOH and centrifuged. The supernatant was then used for first measuring the abundance of total [1-14C] signal (derived from both 1-14C-2-deoxy-D-glucose, 1-14C-2-deoxy-D-glucose 6 phosphate) and, following a precipitation step with 0.3 N Ba(OH)₃ and 0.3 N ZnSO₄, for the measuring of non-phosphorylated 1-14C-2-deoxy-D-glucose. Glycogen was isolated by ethanol precipitation from 30% KOH tissue lysates, as described (Shiota, 2012). Insulin level in plasma at T₀ and T₁₄₀ were measured using a Stellux ELISA rodent insulin kit (Alpco).

### Quantitative RT-PCR

3T3-F442A preadipocytes cells treated with either vehicle (DMSO) or 10 μM SN-401 for 96 h were solubilized in TRIzol and the total RNA was isolated using Purelink RNA kit (Life Technologies). The cDNA synthesis, qRT-PCR reaction and quantification were carried out as described previously (Zhang et al., 2017).

### Liver Isolation, Triglycerides and Histology

HFD mice treated with either vehicle or SN-401 were anesthetized with 1-4% isoflurane followed by cervical dislocation. Gross liver weights were measured and identical sections from right medial lobe of liver were dissected for further examinations. Total triglyceride content was determined by homogenizing 10-50 mg of tissue in 1.5 ml of chloroform:methanol (2:1 v/v) and centrifuged at 12000 rpm for 10 mins at 4° C. An aliquot, 20 ul, was evaporated in a 1.5 ml microcentrifuge tube for 30 mins. Triglyceride content was determined by adding 100 μl of Infinity Triglyceride Reagent (Fisher Scientific) to the dried sample followed by 30 min incubation at RT. The samples were then transferred to a 96 well plate along with standards (0-2000 mg/di) and absorbance was measured at 540 nm and the final concentration was determined by normalizing to tissue weight. For histological examination, liver sections were fixed in 10% zinc formalin and paraffin embedded for sectioning. Hematoxylin and eosin (H&E) stained sections were then assessed for steatosis grade, lobular inflammation and hepatocyte ballooning for non-alcoholic fatty liver disease (NAFLD) scoring as described (Kleiner et al., 2005; Liang et al., 2014; Rauckhorst et al., 2017).

### Quantification and Statistical Analysis

Standard unpaired or paired two-tailed Student's t-test were performed while comparing two groups. One-way Anova was used for multiple group comparison. For GTTs and ITTs, 2-way analysis of variance (Anova) was used. A p-value less than 0.05 was considered statistically significant. *, ** and *** represents a p-value less than 0.05, 0.01 and 0.001 respectively. All data are represented as mean±SEM. All statistical details and analysis are indicated in the brief descriptions of the figures.

### Example 5: Synthesis

General Information: All commercially available reagents and solvents were used directly without further purification unless otherwise noted. Reactions were monitored either by thin-layer chromatography (carried out on silica plates, silica gel 60 F2s4, Merck) and visualized under UV light. Flash chromatography was performed using silica gel 60 as stationary phase performed under positive air pressure. 1H NMR spectra were recorded in CDCb on a Bruker Avance spectrometer operating at 300 MHz at ambient temperature unless otherwise noted. All peaks are reported in ppm on a scale downfield from TMS and using the residual solvent peak in CDCb (H 5=7.26) or TMS (5=0.0) as an internal standard. Data for 1H NMR are reported as follows: chemical shift (ppm, scale), multiplicity (s=singlet, d=doublet, t=triplet, q=quartet, m=multiplet and/or multiplet resonances, dd=double of doublets, dt=double of triplets, br=broad), coupling constant (Hz), and integration. All high-resolution mass spectra (HRMS) were measured on Waters Q-Tof Premier mass spectrometer using electrospray ionization (ESI) time-of-flight (TOF).

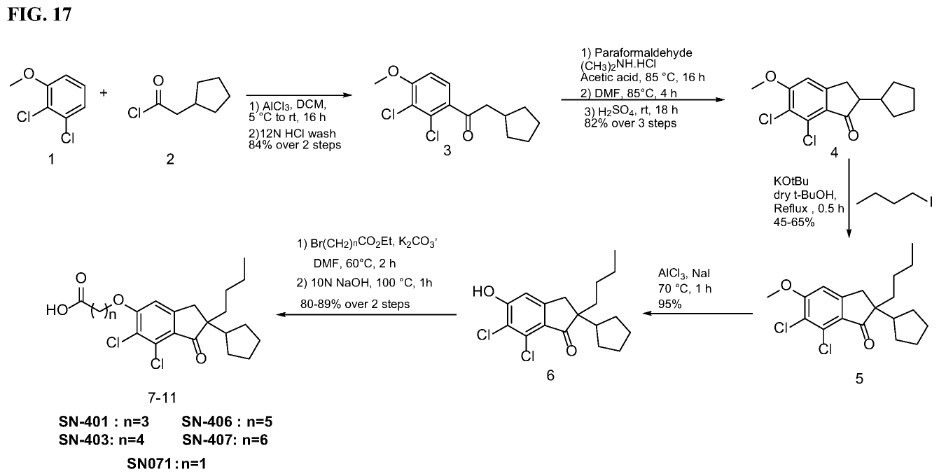

2-cyclopentyl-1-(2,3-dichloro-4-methoxyphenyl)ethan-1-one (3) was prepared according to Scheme 1 (FIG. 17).

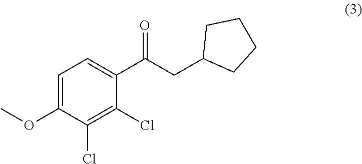 [SMILES: COc1ccc(C(=O)CC2CCCC2)c(Cl)c1Cl]

To a stirring solution of aluminum chloride (13.64 g, 102 mmol, 1.1 equiv.) in dichloromethane (250 ml) at 0° C. was added cyclopentyl acetyl chloride (15 g, 102 mmol, 1.1 equiv.) and the resulting solution was allowed to stir at 0° C. under nitrogen atmosphere for 10 minutes. To this was added a solution of 2,3-dicholoro anisole (16.46 g, 92.9 mmol, 1 equiv.) in dichloromethane (50 ml) at 0° C. and the resulting solution was allowed to warm to room temperature and stirred for 16 hours. Once complete, the reaction was added to cold concentrated hydrochloric acid (100 ml) followed by extraction in dichloromethane (150 ml×3). The organic fractions were pooled, concentrated and purified by silica gel chromatography using 0-15% ethyl acetate in hexanes as eluent to furnish compound 3 as white solid (22.41 g, 84%). NMR (300 MHz, CDCl₃) δ 7.39 (d, J=8.7 Hz, 1H), 6.89 (d, J=8.7 Hz, 1H), 3.96 (s, 3H), 2.96 (d, J=7.2 Hz, 2H), 2.38-2.21 (m, 1H), 1.92-1.75 (m, 2H), 1.69-1.46 (m, 4H), 1.28-1.05 (m, 2H). HRMS (ESI), m/z calcd for C₁₄H₁₇Cl₂O₂ [M+H]⁺ 287.0605, found 287.0603.

6,7-dichloro-2-cyclopentyl-5-methoxy-2,3-dihydro-1H-inden-1-one (4) was prepared according to Scheme 1 (FIG. 17).

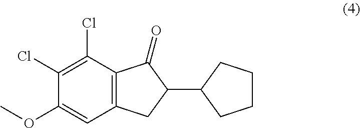 [SMILES: COc1cc2c(c(Cl)c1Cl)C(=O)C(C1CCCC1)C2]

To 2-cyclopentyl-1-(2,3-dichloro-4-methoxyphenyl)ethan-1-one (3) (21.5 g, 74.8 mmol, 1 equiv.) in a round bottom flask was added paraformaldehyde (6.74 g, 224.5 mmol, 3 equiv.), dimethylamine hydrochloride (30.52 g, 374 mmol, 5 equiv.) and acetic acid (2.15 ml) and the resulting mixture was allowed to stir at 85° C. for 16 hours. To the reaction was then added dimethylformamide (92 ml) and the resulting solution was allowed to stir at 85° C. for 4 hours. Once complete, the reaction was diluted with ethyl acetate and then washed with 1N hydrochloric acid. The organic fractions were collected and concentrated under vacuum and used for next step without purification. To the concentrated product in a round bottom flask was added cold concentrated sulfuric acid (120 ml) at 0° C. and the resulting solution was allowed to stir at room temperature for 18 hours. Once complete, the reaction was diluted with cold water and extracted thrice with ethyl acetate (100 ml). The organic fractions were pooled, concentrated and purified by silica gel chromatography using 0-15% ethyl acetate in hexanes as eluent to furnish compound 4 as beige solid (18.36 g, 82%). NMR (300 MHz, CDCl₃) δ 6.88 (s, 1H), 4.00 (s, 3H), 3.16 (dd, J=18.1, 8.7 Hz, 1H), 2.80 (d, J=14.4 Hz, 2H), 2.43-2.22 (m, 1H), 1.96 (s, 1H), 1.73-1.48 (m, 5H), 1.46-1.33 (m, 1H), 1.17-1.00 (m, 1H). LRMS (ESI), m/z calcd for C₁₅H₁₇Cl₂O₂ [M+H]⁺ 299.0605, found 299.0614.

2-butyl-6,7-dichloro-2-cyclopentyl-5-methoxy-2,3-dihydro-1H-inden-1-one (5) was prepared according to Scheme 1 (FIG. 17).

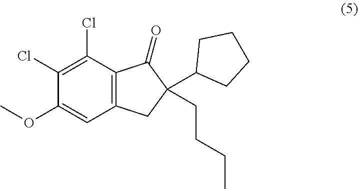 [SMILES: CCCCC1(C2CCCC2)Cc2cc(OC)c(Cl)c(Cl)c2C1=O]

A stirring suspension of 4 (23 gm, 76.8 mmol, 1 equiv.) in anhydrous tert-butanol (220 ml) was allowed to reflux at 95° C. for 30 minutes. To the resulting solution was added potassium tert-butanol (1M in tert-butanol) (84 ml, 84.5 mmol, 1.1 equiv.) and the resulting solution was refluxed for 30 minutes. The reaction was then cooled to room temperature followed by addition of iodobutane (44.2 ml, 384 mmol, 5 equiv.) and the reaction was then allowed to reflux for additional 60 minutes. The reaction was allowed to cool, concentrated and purified by silica gel chromatography using 0-10% ethyl acetate in hexanes as eluent to furnish compound 5 as clear oil (17.75 g, 65%). NMR (300 MHz, CDCl₃) δ 6.89 (s, 1H), 4.09-3.90 (m, 3H), 2.98-2.70 (m, 2H), 2.36-2.18 (m, 1H), 1.89-1.71 (m, 2H), 1.58-1.42 (m, 5H), 1.33-1.09 (m, 4H), 1.09-0.94 (m, 2H), 0.93-0.73 (m, 4H). HRMS (ESI), m/z calcd for C₁₉H₂₅Cl₂O₂ [M+H]⁺ 355.1231, found 355.1231.

2-butyl-6,7-dichloro-2-cyclopentyl-5-hydroxy-2,3-dihydro-1H-inden-1-one (6) was prepared according to Scheme 1 (FIG. 17).

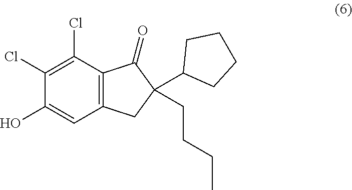 [SMILES: CCCCC1(C2CCCC2)Cc2cc(O)c(Cl)c(Cl)c2C1=O]

To 5 (3.14 g, 8.87 mmol, 1 equiv.) was added aluminum chloride (2.36 g, 17 mmol, 2 equiv.) and sodium iodide (2.7 g, 17 mmol, 2 equiv.) and the resulting solid mixture was triturated and allowed to stir at 70° C. for 60 minutes. Once complete, the reaction was diluted with dichloromethane and washed with aqueous saturated sodium thiosulfate solution. The organic fractions were collected and concentrated to give a beige solid which was then washed multiple times with hexanes to provide compound 6 as white solid (2.87 g, 95%). NMR (300 MHz, CDCl₃) δ 7.03 (s, 1H), 6.32 (s, 1H), 2.97-2.73 (m, 2H), 2.36-2.17 (m, 1H), 1.88-1.68 (m, 2H), 1.62-1.39 (m, 6H), 1.31-1.11 (m, 3H), 1.08-0.97 (m, 2H), 0.97-0.87 (m, 1H), 0.83 (t, J=7.3 Hz, 3H). HRMS (ESI), m/z calcd for C₁₈H₂₃Cl₂O₂ [M+H]⁺ 341.1075, found 341.1089.

2-((2-butyl-6,7-dichloro-2-cyclopentyl-1-oxo-2,3-dihydro-1H-inden-5-yl)oxy)acetic acid (7) (SN071) was prepared according to Scheme 1 (FIG. 17).

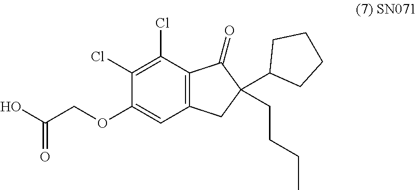 [SMILES: CCCCC1(C2CCCC2)Cc2cc(OCC(=O)O)c(Cl)c(Cl)c2C1=O]

To a stirring solution of 5 (170 mg, 0.50 mmol, 1 equiv.) in anhydrous dimethylformamide (1 ml) was added potassium carbonate (76 mg, 0.56 mmol, 1.1 equiv.) and ethyl 2-bromoacetate (61 μl, 0.56 mmol, 1.1 equiv.) and the reaction was allowed to stir at 60° C. for 2 hours. Once complete, to the reaction was added 4 N NaOH (1 ml) and the reaction was allowed to stir at 100° C. for 60 minutes. Once complete, reaction was concentrated and purified by column chromatography using 0-10% methanol in dichloromethane as eluent to provide SN071 as a clear solid (173 mg, 87%). NMR (300 MHz, CDCl₃) δ 6.80 (s, 1H), 5.88 (s, 1H), 4.88 (s, 2H), 2.87 (q, J=17.9 Hz, 2H), 2.34-2.20 (m, 1H), 1.91-1.69 (m, 2H), 1.66-1.39 (m, 6H), 1.32-1.13 (m, 3H), 1.10-0.95 (m, 2H), 0.94-0.86 (m, 1H), 0.83 (t, J=7.3 Hz, 3H). HRMS (ESI), m/z calcd for C₂₀H₂₅Cl₂O₄ [M+H]⁺ 399.1130, found 399.1132.

4-((2-butyl-6,7-dichloro-2-cyclopentyl-1-oxo-2,3-dihydro-1H-inden-5-yl)oxy)butanoic acid (8) (SN-401) was prepared according to Scheme 1 (FIG. 17).

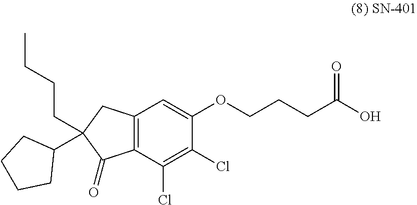 [SMILES: CCCCC1(C2CCCC2)Cc2cc(OCCCC(=O)O)c(Cl)c(Cl)c2C1=O]

To a stirring solution of 5 (100 mg, 0.29 mmol, 1 equiv.) in anhydrous dimethylformamide (1 ml) was added potassium carbonate (45 mg, 0.32 mmol, 1.1 equiv.) and ethyl 4-bromobutyrate (46 μl, 0.32 mmol, 1.1 equiv.) and the reaction was allowed to stir at 60° C. for 2 hours. Once complete, to the reaction was added 4 N NaOH (1 ml) and the reaction was allowed to stir at 100° C. for 60 minutes. Once complete, reaction was concentrated and purified by column chromatography using 0-10% methanol in dichloromethane as eluent to provide SN-401 as a clear solid (111 mg, 89%). NMR (300 MHz, CDCl₃) δ 10.77 (s, 1H), 6.86 (s, 1H), 4.21 (t, J=5.9 Hz, 2H), 2.88 (t, J=14.4 Hz, 2H), 2.69 (t, J=7.0 Hz, 2H), 2.26 (dd, J=12.6, 6.1 Hz, 3H), 1.87-1.73 (m, 2H), 1.64-1.44 (m, 6H), 1.35-1.10 (m, 4H), 1.08-0.95 (m, J=15.0, 7.7 Hz, 2H), 0.82 (t, J=7.3 Hz, 3H). HRMS (ESI), m/z calcd for C22H29C1204 [M+H]+427.1443, found 427.1446.

5-((2-butyl-6,7-dichloro-2-cyclopentyl-1-oxo-2,3-dihydro-1H-inden-5-yl)oxy)pentanoic acid (9) (SN-403) was prepared according to Scheme 1 (FIG. 17).

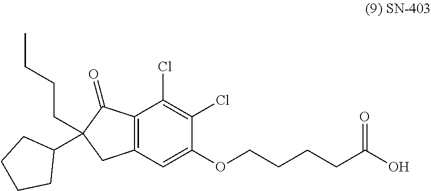 [SMILES: CCCCC1(C2CCCC2)Cc2cc(OCCCCC(=O)O)c(Cl)c(Cl)c2C1=O]

To a stirring solution of 5 (100 mg, 0.29 mmol, 1 equiv.) in anhydrous dimethylformamide (1 ml) was added potassium carbonate (45 mg, 0.32 mmol, 1.1 equiv.) and ethyl 6-bromovalerate (51 μl, 0.32 mmol, 1.1 equiv.) and the reaction was allowed to stir at 60° C. for 2 hours. Once complete, to the reaction was added 4 N NaOH (1 ml) and the reaction was allowed to stir at 100° C. for 60 minutes. Once complete, reaction was concentrated and purified by column chromatography using 0-10% methanol in dichloromethane as eluent to provide SN-403 as a clear solid (114 mg, 88%). NMR (300 MHz, CDCl₃) δ 10.95 (s, 1H), 6.85 (brs, 1H), 4.16 (t, J=5.7 Hz, 2H), 2.96-2.75 (m, 2H), 2.61-2.44 (m, 2H), 2.35-2.17 (m, 1H), 2.10-1.87 (m, 4H), 1.86-1.70 (m, 2H), 1.66-1.38 (m, 6H), 1.32-1.13 (m, 3H), 1.08-0.96 (m, 2H), 0.94-0.86 (m, 1H), 0.86-0.73 (m, 3H). HRMS (ESI), m/z calcd for C₂₃H₃₁Cl₂O₄ [M+H]⁺ 441.1599, found 441.1601.

### 6-((2-butyl-6,7-dichloro-2-cyclopentyl-1-oxo-2,3-dihydro-1H-inden-5-yl)oxy)hexanoic acid (10) (SN-406) was Prepared According to Scheme 1 (FIG. 17)

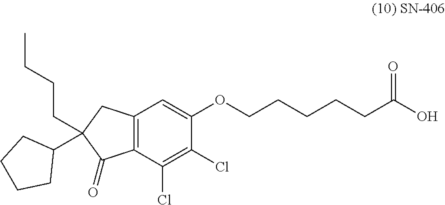 [SMILES: CCCCC1(C2CCCC2)Cc2cc(OCCCCCC(=O)O)c(Cl)c(Cl)c2C1=O]

To a stirring solution of 5 (100 mg, 0.29 mmol, 1 equiv.) in anhydrous dimethylformamide (1 ml) was added potassium carbonate (45 mg, 0.32 mmol, 1.1 equiv.) and ethyl 6-bromohexanoate (58 μl, 0.32 mmol, 1.1 equiv.) and the reaction was allowed to stir at 60° C. for 2 hours. Once complete, to the reaction was added 4 N NaOH (1 ml) and the reaction was allowed to stir at 100° C. for 60 minutes. Once complete, reaction was concentrated and purified by column chromatography using 0-10% methanol in dichloromethane as eluent to provide SN-406 as a clear solid (115 mg, 86%). 1H NMR (300 MHz, CDCl3) δ 11.70 (s, 1H), 6.85 (s, 1H), 4.13 (t, J=6.2 Hz, 2H), 2.93-2.74 (m, 2H), 2.43 (t, J=7.3 Hz, 2H), 2.32-2.17 (m, 1H), 1.98-1.87 (m, 2H), 1.85-1.68 (m, 4H), 1.66-1.40 (m, 8H), 1.28-1.12 (m, 3H), 1.07-0.93 (m, 2H), 0.91-0.70 (m, 4H). HRMS (ESI), m/z calcd for C24H33Cl2O4 [M+H]+ 455.1756, found 455.1756.

7-((2-butyl-6,7-dichloro-2-cyclopentyl-1-oxo-2,3-dihydro-1H-inden-5-yl)oxy)heptanoic acid (11) (SN-407) was prepared according to Scheme 1 (FIG. 17).

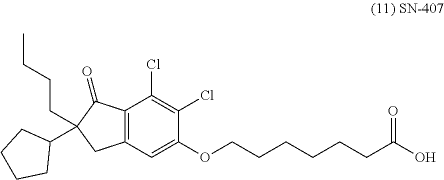 [SMILES: CCCCC1(C2CCCC2)Cc2cc(OCCCCCCC(=O)O)c(Cl)c(Cl)c2C1=O]

To a stirring solution of 5 (100 mg, 0.29 mmol, 1 equiv.) in anhydrous dimethylformamide (1 ml) was added potassium carbonate (45 mg, 0.32 mmol, 1.1 equiv.) and ethyl 7-bromoheptanoate (63 μl, 0.32 mmol, 1.1 equiv.) and the reaction was allowed to stir at 60° C. for 2 hours. Once complete, to the reaction was added 4 N NaOH (1 ml) and the reaction was allowed to stir at 100° C. for 60 minutes. Once complete, reaction was concentrated and purified by column chromatography using 0-10% methanol in dichloromethane as eluent to provide SN-407 as a clear solid (122 mg, 89%). NMR (300 MHz, CDCl₃) δ 11.52 (s, 1H), 6.85 (s, 1H), 4.12 (t, J=6.3 Hz, 2H), 2.84 (q, J=18.2 Hz, 2H), 2.47-2.32 (m, 2H), 2.32-2.18 (m, 1H), 1.96-1.84 (m, 2H), 1.83-1.64 (m, 4H), 1.62-1.39 (m, 10H), 1.28-1.14 (m, 3H), 1.08-0.94 (m, 2H), 0.91 (d, J=8.5 Hz, 1H), 0.81 (t, J=7.3 Hz, 3H). HRMS (ESI), m/z calcd for C₂₅H₃₅Cl₂O₄ [M+H]⁺ 469.1912, found 469.1896.

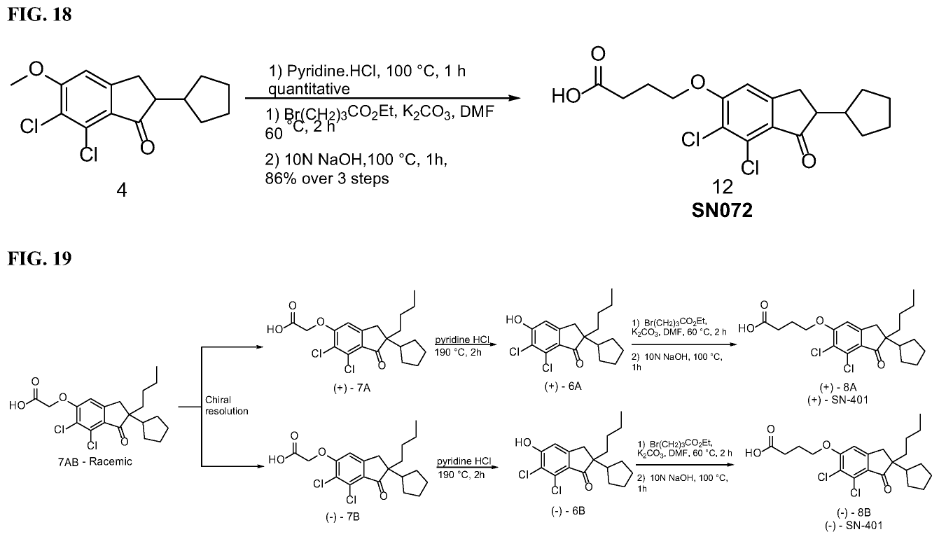

4-((6,7-dichloro-2-cyclopentyl-1-oxo-2,3-dihydro-1H-inden-5-yl)oxy)butanoic acid (12) (SN072) was synthesized according to Scheme 2 (FIG. 18):

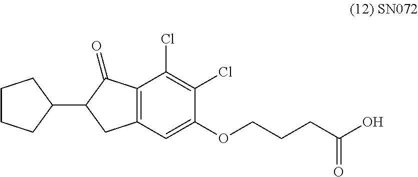 [SMILES: O=C(O)CCCOc1cc2c(c(Cl)c1Cl)C(=O)C(C1CCCC1)C2]

To 4 (100 mg, 0.36 mmol, 1 equiv.) was added aluminum chloride (89 mg, 0.67 mmol, 2 equiv.) and sodium iodide (101 mg, 0.67 mmol, 2 equiv.) and the resulting solid mixture was triturated and allowed to stir at 70° C. for 60 minutes. Once complete, the reaction was diluted with dichloromethane and washed with aqueous saturated sodium thiosulfate solution. The organic fractions were collected and concentrated to give a beige solid which was then washed multiple times with hexanes to provide compound 6 as white solid which was used for the next step. To a stirring solution of the product form the first step in anhydrous dimethylformamide (1 ml) was added potassium carbonate (53 mg, 0.39 mmol, 1.1 equiv.) and ethyl 4-bromobutyrate (55 μl, 0.39 mmol, 1.1 equiv.) and the reaction was allowed to stir at 60° C. for 2 hours. Once complete, to the reaction was added 4 N NaOH (1 ml) and the reaction was allowed to stir at 100° C. for 60 minutes. Once complete, reaction was concentrated and purified by column chromatography using 0-10% methanol in dichloromethane as eluent to provide SN072 as a clear solid (107 mg, 86%). ¹H NMR (300 MHz, CDCl₃) δ 6.87 (s, 1H), 4.21 (t, J=5.9 Hz, 2H), 3.26-3.02 (m, 1H), 2.94-2.56 (m, 4H), 2.40-2.19 (m, 3H), 2.03-1.90 (m, 1H), 1.74-1.50 (m, 5H), 1.47-1.32 (m, 1H), 1.19-1.00 (m, 1H). HRMS (ESI), m/z calcd for C₁₈H₂₁Cl₂O₄ [M+H]⁺ 371.0817, found 371.0808.

Enantiomerically enriched SN-401 isomers were synthesized following literature reported procedure (Cragoe et al., 1982) and as depicted in scheme 3, FIG. 19. In brief, racemic compound 7 (1 equiv.) was dissolved along with cinchonine (1 equiv.) in minimum amount of hot DMF and the allowed to cool. The precipitated salt was separated (filtrate used to obtain opposite enantiomer) and recrystallized 5 additional times from DMF, followed by acidification of salt with aqueous HCl and extraction into ether. The ether was evaporated under vacuum to furnish the enantiomerically enriched (+)-7A in 23% yield; [α]25D +16.8° (c 5, EtOH). The DMF filtrate from the first step now enriched in (−)-7B was concentrated and acidified with aqueous HCl and extracted in ether and concentrated to give solid. This resulting solid (1 equiv.) was dissolved with cinchonidine (1 equiv.) in minimum amount of hot ethanol and then allowed to cool. The precipitated salt was separated and recrystallized 5 additional times from DMF, followed by acidification of salt with aqueous HCl and extraction into ether. The ether was evaporated under vacuum to furnish enantiomerically enriched (−)-7A in 19% yield; [α]25D −15.6° (c 5, EtOH). The enantiomerically enriched 7A and 7B were then subjected to same two step reaction sequence involving transformation to respective phenols (+)-6A and (−)-6B followed by conversion to desired enantiomerically enriched oxybutyric acids (+)-8A [α]^{25D} +15.9° (c 5, EtOH) and (−)-8B [α]^{25D} −14.5° (c 5, EtOH). The 1H NMR and HRMS for enantiomerically enriched products are same as racemic compounds and thus not reported.

### Example 6: I_{Cl,SWELL} and SWELL1 Protein are Reduced in T2D β-Cells and Adipocytes

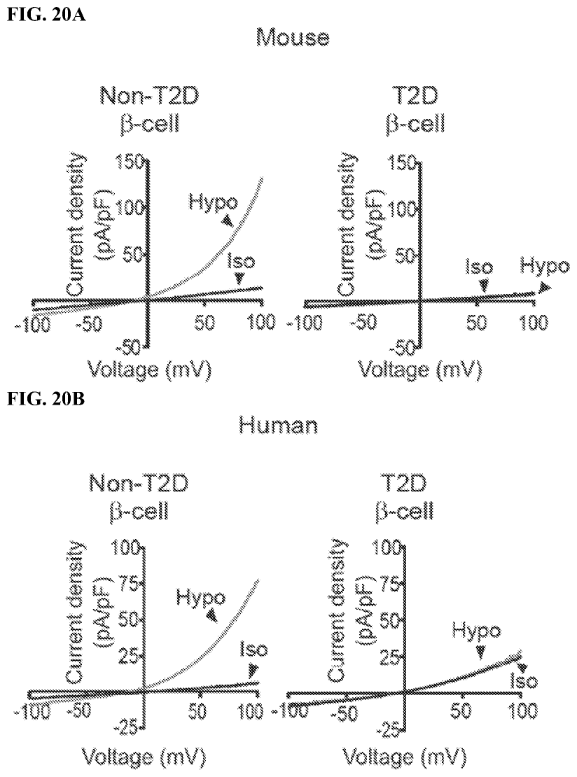

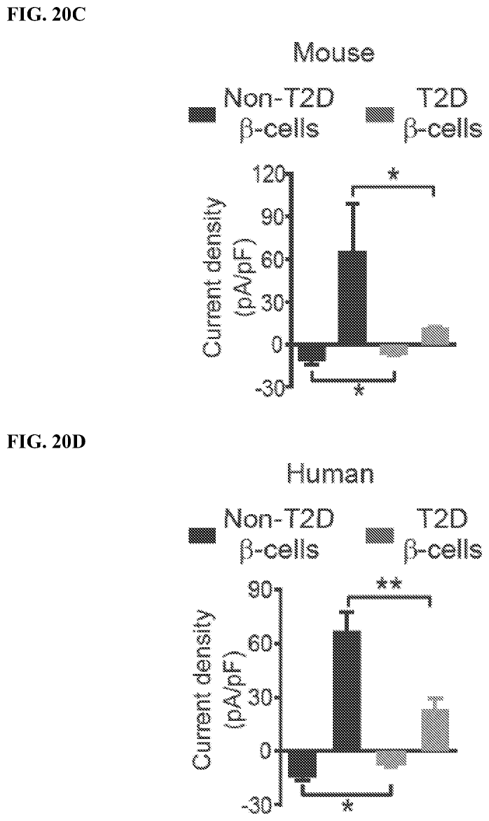

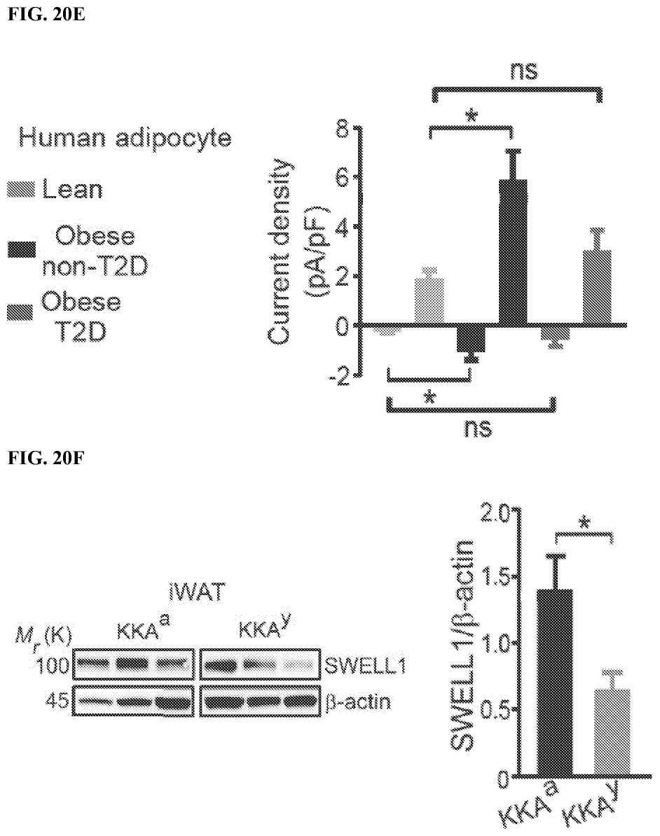

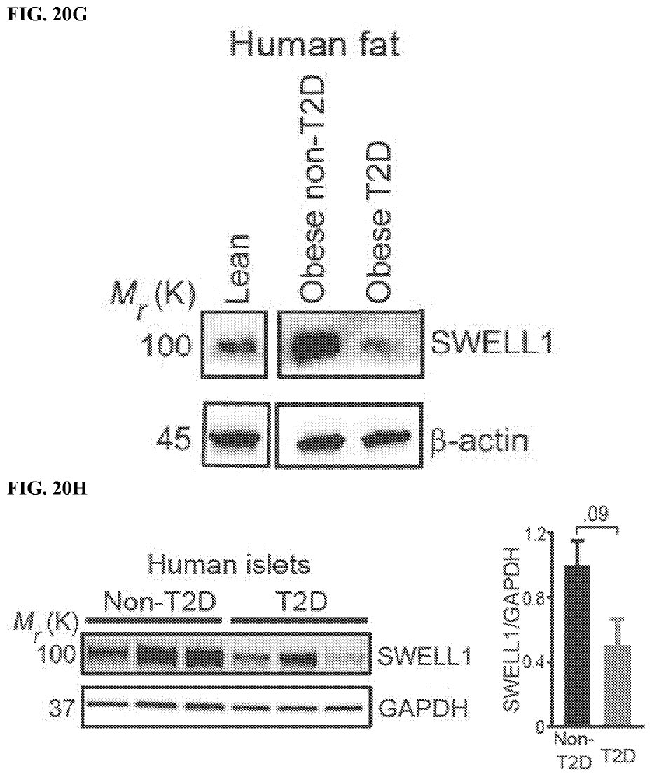

SWELL1/LRRC8a ablation impairs insulin signaling in target tissues and insulin secretion from the pancreatic β3-cell, inducing a pre-diabetic state of glucose intolerance. These recent findings show that reductions in SWELL1 may contribute to Type 2 diabetes (T2D). To determine if SWELL1-mediated currents are altered in T2D we measured I_{Cl,SWELL} in pancreatic β-cells freshly isolated from T2D mice raised on HFD for 5-7 months (FIG. 20A) and from T2D patients (FIG. 20B, Tables 2 and 3, below) compared to non-T2D controls. In both mouse and human T2D β-cells, the maximum I_{Cl,SWELL} current density (measured at +100 mV) upon stimulation with hypotonic swelling is significantly reduced (83% in murine; 63% in human, FIGS. 20C and 20D) compared to non-T2D controls, similar to reductions observed in SWELL1 knock-out (KO) and knock-down (KO) murine and human β-cells (Kang et al., 2018), respectively. These reductions in β-cell I_{Cl,SWELL} in the setting of T2D are consistent with previous measurements of VRAC/I_{Cl,SWELL} in the murine KKN T2D model, which were reduced by >50% compared to I_{Cl,SWELL} in adipocytes isolated from T2D KKN mice compared to non-T2D controls. Likewise, SWELL1-mediated I_{Cl,SWELL} measured in isolated human adipocytes from an obese T2D patient (BMI=52.3, HgbA1c=6.9%; Fasting Glucose=148-151 mg/di) show a trend toward being reduced 50% compared to obese, non-T2D patients that we reported previously, and not different from I_{Cl,SWELL} in adipocytes from lean patients (FIG. 20E, Table 4, below). As SWELL1/LRRC8a is a critical component of I_{Cl,SWELL} IV RAC in both adipose tissue, we asked whether these reductions in I_{Cl,SWELL} in the setting of T2D are associated with reductions in SWELL1 protein expression. Indeed, SWELL1 protein is reduced in adipose tissue of T2D KKN mice as compared to parental control KKAa mice (FIG. 20F). Similarly, SWELL1 protein is lower in adipose tissue from an obese T2D patient (BMI=53.7, HgbA1c=8.0%, Fasting Glucose=183-273 mg/di) compared to adipose tissue from a normoglycemic obese patient (BMI=50.2 HgbA1c=5.0%; Fasting Glucose=84-97 mg/di, FIG. 20G, Table 5, below). Moreover, total SWELL1 protein in diabetic human cadaveric islets shows a trend toward being reduced 50% compared to islets from non-diabetics (FIG. 20H, Table 6, below). Taken together, these findings show that reduced SWELL1 activity in adipocytes and β-cells (and possibly other tissues) may underlie insulin-resistance and impaired insulin secretion associated with T2D. Moreover, SWELL1 protein expression increases in both adipose tissue and liver in the setting of early euglycemic obesity and shRNA-mediated suppression of this SWELL1 induction exacerbates insulin-resistance and glucose intolerance. Therefore, we speculate that maintenance or induction of SWELL1 expression/signaling in peripheral tissues may support insulin sensitivity and secretion to preserve systemic glycemia in the setting of T2D.

TABLE 2
Characteristics of non-T2D and T2D mice from which β-cells
were isolated for patch-claim studies in FIGS. 20A and 20C

| | Age | | | Body | Glucose |
|---|---|---|---|---|---|
| Mouse | (weeks) | Sex | Diet | Mass (g) | (mg/dl) |
| Non-T2D | 12-13 (n = 4) | M | Regular Chow | 28.8 +/− 0.51 | 148 +/6.49 |
| T2D | 23-27 (n = 3) | M | High-fat diet | 52.7 +/− 2.99 | 229 +/− 21.4 |

TABLE 3
Characteristic of patients from whom cadaveric
non-T2D and T2D islets were obtained for β-cell
patch-clamp studies in FIGS. 20B and 20D.

| | | | | Random | Estimated | |
|---|---|---|---|---|---|---|
| | Age | | | Glucose | Glucose | HbA1C |
| Patient | (years) | Sex | BMI | (mg/dl) | (mg/dl) | (%) |
| Non- | 44 | F | 26.8 | 151.8 | NA | 6.1 |
| T2D | 57 | M | 28.7 | 144.3 | NA | 5.3 |
| | 24 | F | 32.2 | 234 | NA | NA |
| T2D | 46 | F | 35.9 | 262.4 | NA | 6.8 |
| | 37 | F | 38.1 | 253.8 | NA | 8.2 |
| | 51 | M | 35.59 | NA | 157 | 7.1 |

(NA: not available)

TABLE 4
Characteristics of lean, non-T2D, and T2D bariatric
surgery patients from whom primary adipocytes were
isolated for patch-clamp studies in FIG. 20E.

| | | | | Random | Estimated | |
|---|---|---|---|---|---|---|
| | Age | | | Glucose | Glucose | HbA1C |
| Patient | (years) | Sex | BMI | (mg/dl) | (mg/dl) | (%) |
| Lean | 52 | M | 27.56 | 97 | 111 | 5.5 |
| | 61 | F | 28.36 | 112 | NA | 5.5 |
| Obese | 38 | F | 55.10 | 88 | 117 | 5.7 |
| non-T2D | 65 | F | 32.02 | 100 | 111 | 5.5 |
| | 51 | F | 48.8 | 97 | 114 | 5.6 |
| Obese-T2D | 41 | F | 52.31 | 148 | 151 | 6.9 |

TABLE 5
Characteristics of lean, obese non-T2D, and obese T2D
patients from whom adipose samples were obtained to
measure SWELL1 protein expression levels in FIG. 20G.

| | | | | Random | Estimated | |
|---|---|---|---|---|---|---|
| | Age | | | Glucose | Glucose | HbA1C |
| Patient | (years) | Sex | BMI | (mg/dl) | (mg/dl) | (%) |
| Lean | 47 | F | 24.85 | 97 | 111 | 5.5 |
| Obese | 48 | F | 50.18 | 84 | 97 | 5.0 |
| non-T2D | | | | | | |
| Obese-T2D | 57 | F | 53.69 | 273 | 183 | 8.0 |

TABLE 6
Characteristics of non-T2D and T2D patients from
whom cadaveric islets were obtained to measure
SWELL1 protein expression levels in FIG. 20H.

| Patient | Age (years) | Sex | BMI | HbA1C (%) |
|---|---|---|---|---|
| Non-T2D | 50 | F | 31.7 | 5.7 |
| | 61 | M | 19.6 | 5.9 |
| | 54 | M | 26.4 | 5.1 |
| T2D | 62 | M | 25.9 | 10 |
| | 48 | F | 30.4 | 7.5 |
| | 54 | F | 24.4 | 7.2 |

### Example 7: SWELL1 Protein Expression Regulates Insulin Stimulated PI3K-AKT2-AS160 Signaling

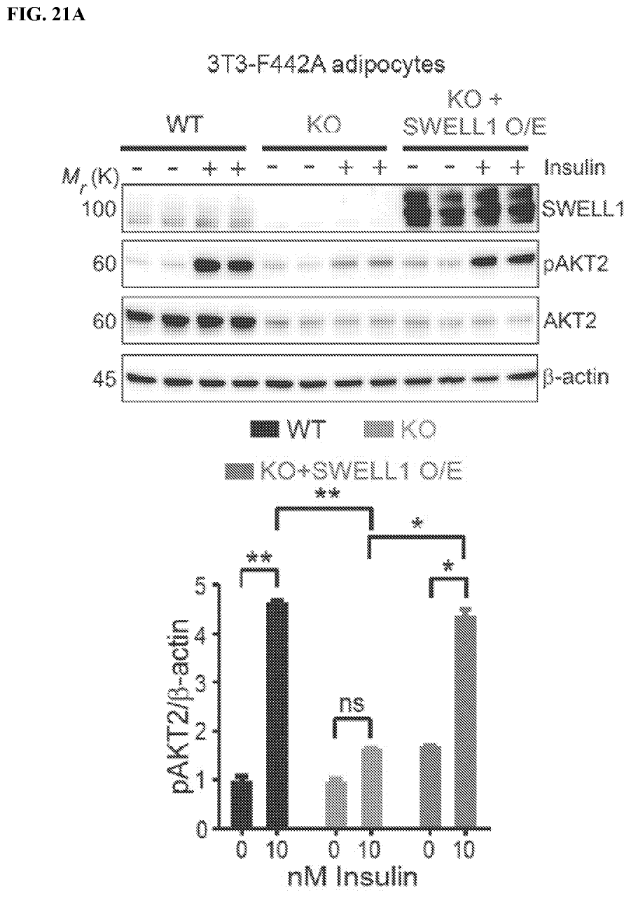

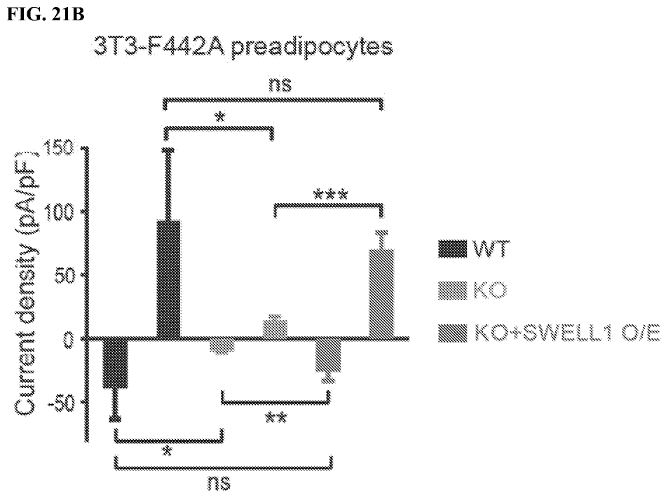

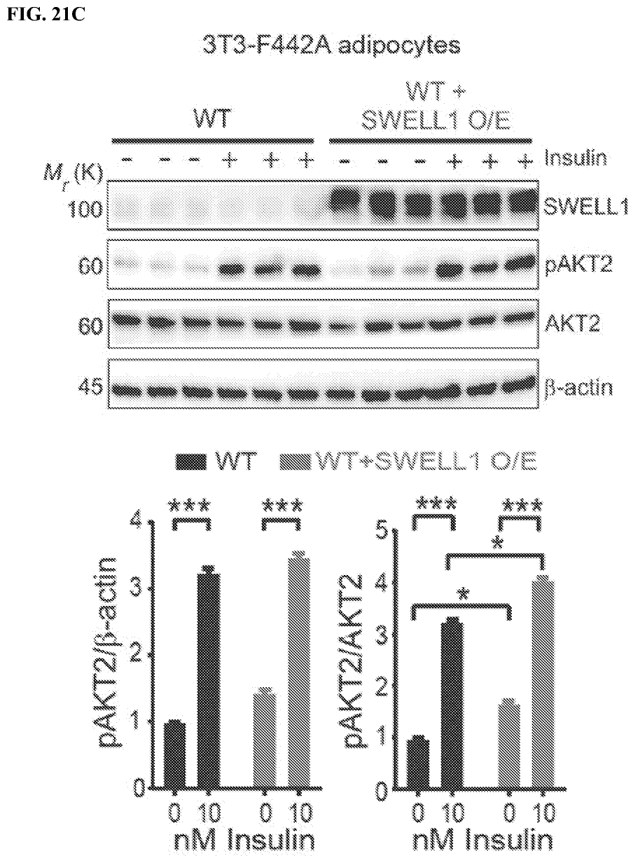

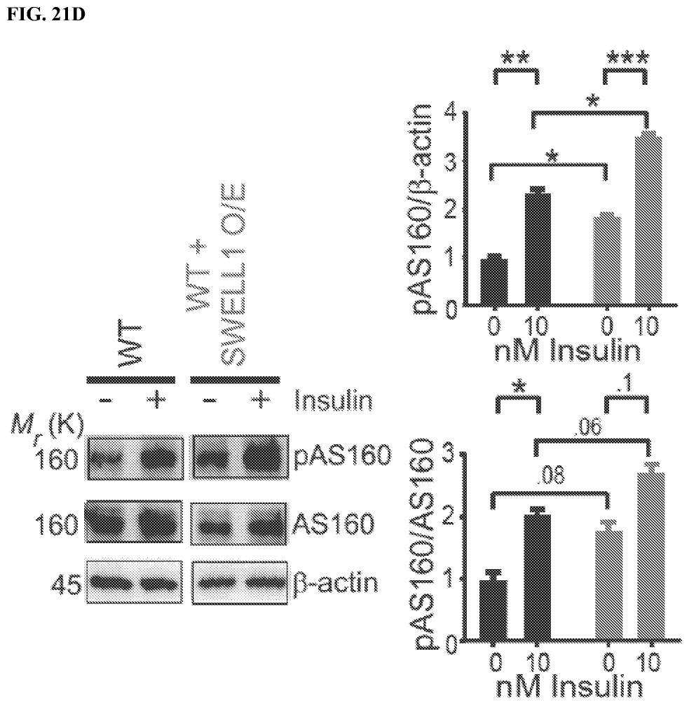

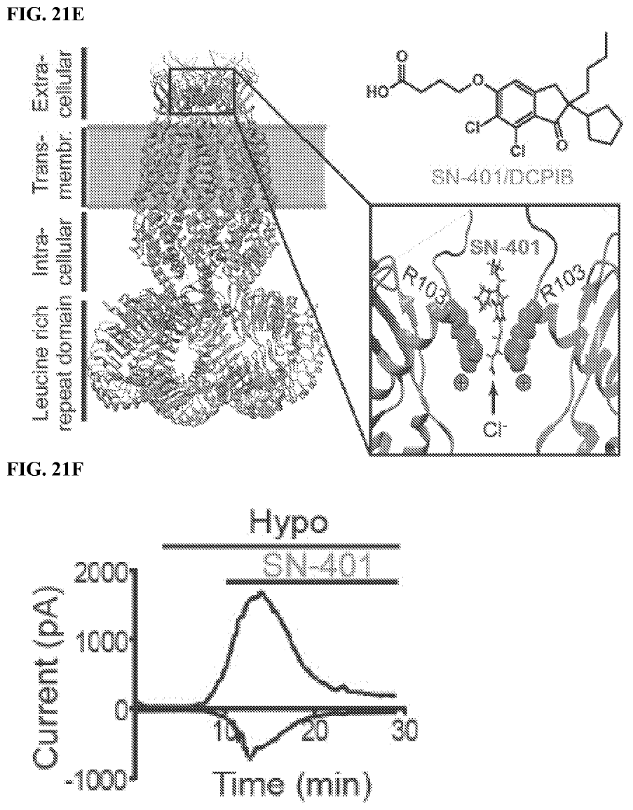

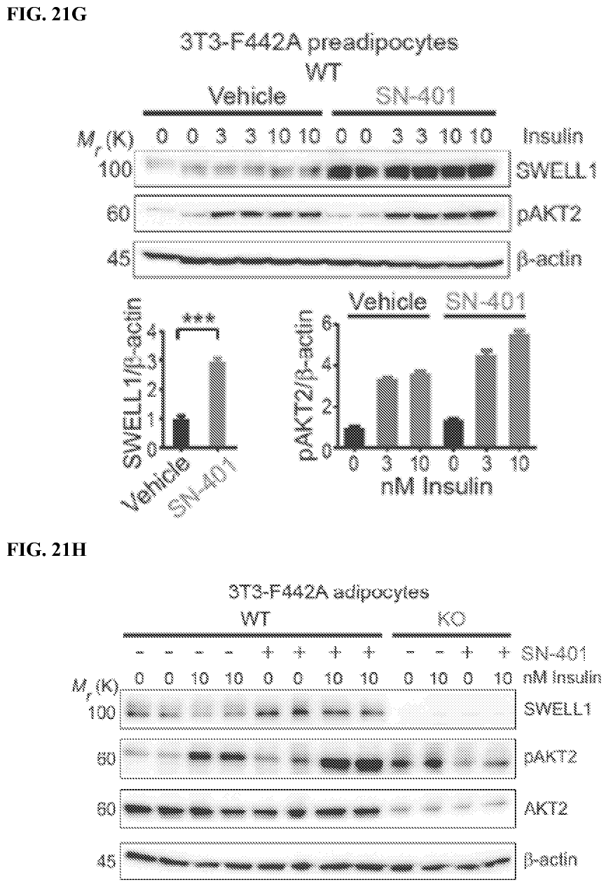

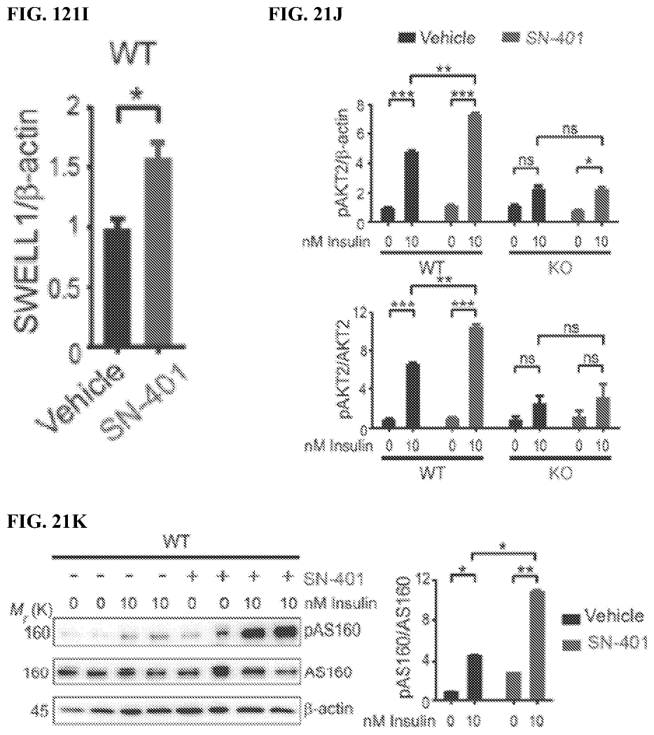

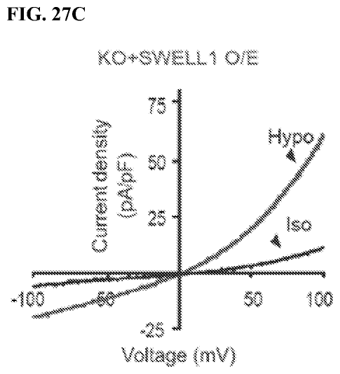

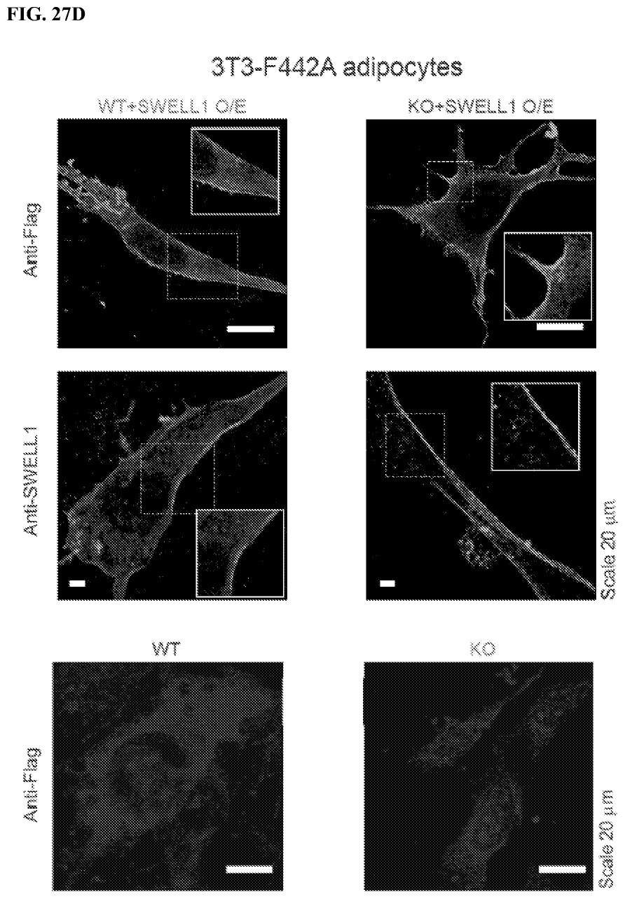

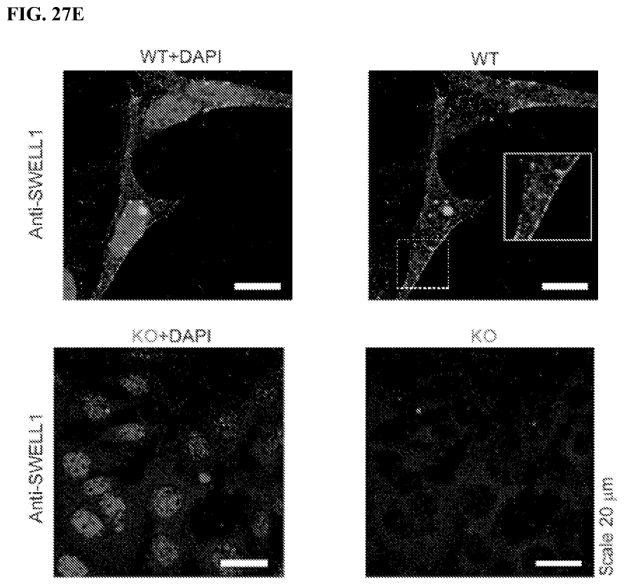

To test whether SWELL1 regulates insulin signaling we overexpressed Flag-tagged SWELL1 (SWELL1 O/E) in both WT and SWELL1 KO 3T3-F442A adipocytes and measured insulin-stimulated phosphorylated AKT2 (pAKT2) as a readout of insulin-sensitivity (FIG. 21A). SWELL1 KO 3T3-F442A adipocytes exhibit significantly blunted insulin-mediated pAKT2 signaling compared to WT adipocytes, as described previously (Zhang et al., 2017), and this is fully rescued by re-expression of SWELL1 in SWELL1 KO adipocytes (KO+SWELL 1 O/E, FIG. 21A), along with restoring SWELL1-mediated I_{Cl,SWELL} in response to hypotonic stimulation (FIG. 21B and FIG. 27A-FIG. 27C), consistent with restoration of SWELL1-LRRC8a signaling complexes at the plasma membrane. Notably, the reductions in total AKT2 protein expression observed in SWELL1 KO adipocytes is not rescued by SWELL1 re-expression, indicating that transient changes in SWELL1 protein expression preferentially regulates insulin-pAKT2 signaling, as opposed to AKT2 protein expression. SWELL1 overexpression in WT adipocytes also increases both basal and insulin-stimulated pAKT2 and downstream phosphorylation of AS160 (pAS160) signaling in WT adipocytes (FIGS. 21C and 21D). We confirmed FLAG-tagged SWELL1 traffics normally to the plasma membrane when expressed in both WT and SWELL1 KO adipocytes visualized by immunofluorescence (IF) using anti-FLAG and SWELL1 KO-validated custom-made anti-SWELL1 antibodies, respectively. FLAG-tagged SWELL1 overexpressed in WT and SWELL1 KO adipocytes assumed a punctate pattern at the cell periphery, similar to endogenous SWELL1 in WT adipocytes (FIGS. 27D and 27E). Overall, these data indicate that SWELL1 expression levels regulate insulin-PI3K-AKT2-AS160 signaling in adipocytes. Furthermore, these data show that pharmacological SWELL1 induction in peripheral tissues in the setting of T2D may enhance insulin signaling, and improve systemic insulin-sensitivity and glucose homeostasis.

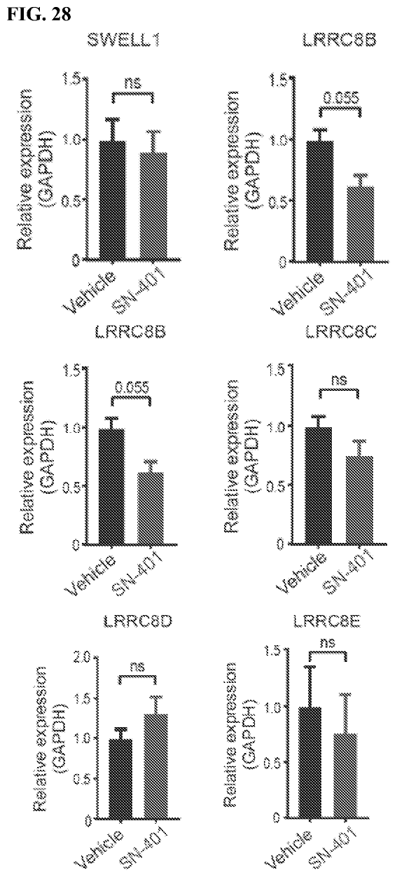

The small molecule 4-[(2-Butyl-6,7-dichloro-2-cyclopentyl-2,3-dihydro-1-oxo-1H-inden-5-yl)oxy]butanoic acid (DCPIB, FIG. 21E) is among a series of structurally diverse (acylaryloxy)acetic acid derivatives, that were synthesized and studied for diuretic properties in the late 1970s and evaluated in the 1980s as potential treatments for brain edema. DCPIB, although derived from the FDA-approved diuretic, ethacrynic acid, has minimal diuretic activity, and has instead been used as a selective VRAC/I_{Cl,SWELL} inhibitor (FIG. 21F), binding at a constriction point within the SWELL1-LRRC8 hexamer (FIG. 21E), with an IC₅₀ of ˜5 μM Having demonstrated that SWELL1 is required for normal insulin signaling in adipocytes, we anticipated pharmacological inhibition of VRAC/I_{Cl,SWELL} with DCPIB, which we here re-name SN-401, would decrease insulin signaling. Unexpectedly, SN-401 increased SWELL1 protein expression in 3T3-F442A preadipocytes (3-fold control expression; FIG. 21G) and adipocytes (1.5-fold control expression; FIG. 21I) when applied for 96 hours, and was associated with enhanced insulin-stimulated levels of pAKT2 (FIGS. 21H and 21J), and insulin-stimulated levels of pAS160 (FIG. 21K). These SN-401-mediated effects on insulin-AKT2-AS160 signaling are absent in SWELL1 KO 3T3-F442A adipocytes, consistent with an on-target SWELL1-mediated mechanism of action for SN-401 (FIGS. 21H and 21J). The SN-401-mediated increases in SWELL1 protein expression are not associated with increases in SWELL1, LRRC8b, LRRC8c, LRRC8d or LRRC8e mRNA expression, implicating a post-transcriptional mechanism for increased expression of these proteins (FIG. 28).

### Example 8: Structure Activity Relationship and Molecular Docking Simulations Reveal Specific SN-401-SWELL1 Interactions Required for On-Target Activity

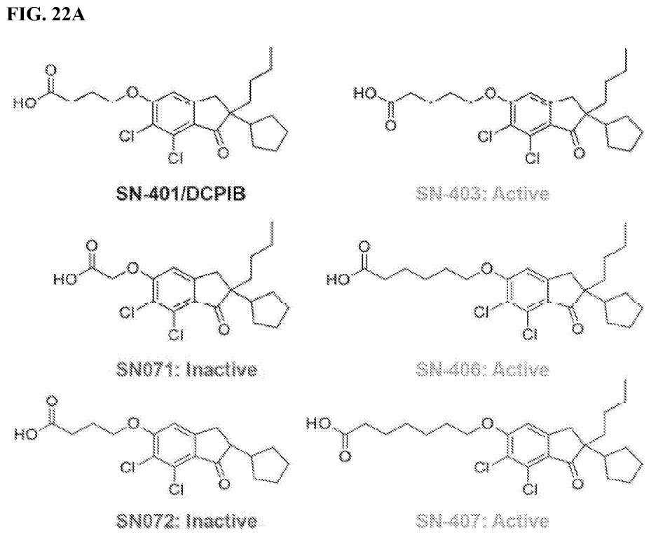

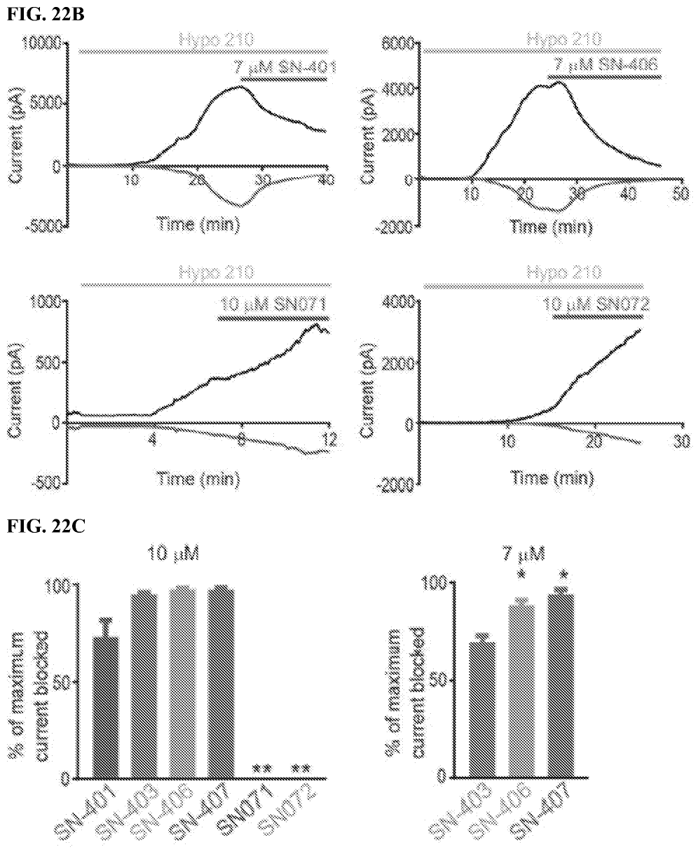

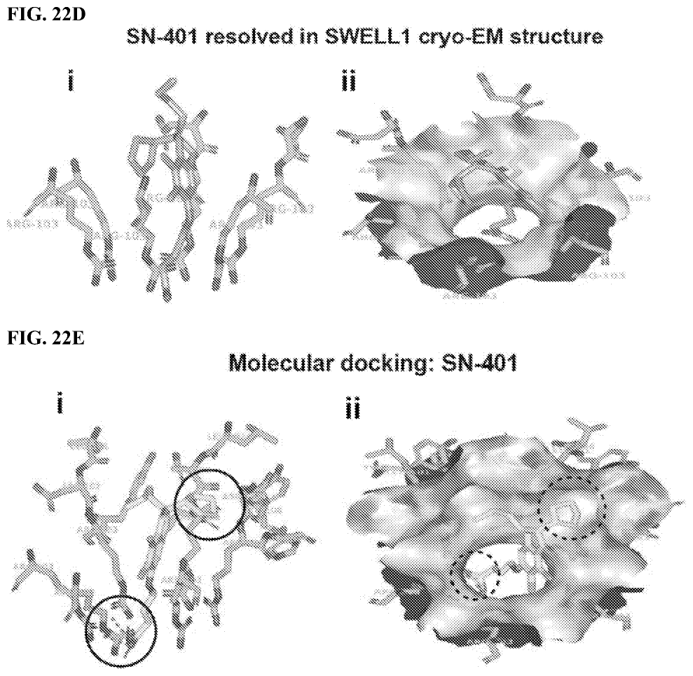

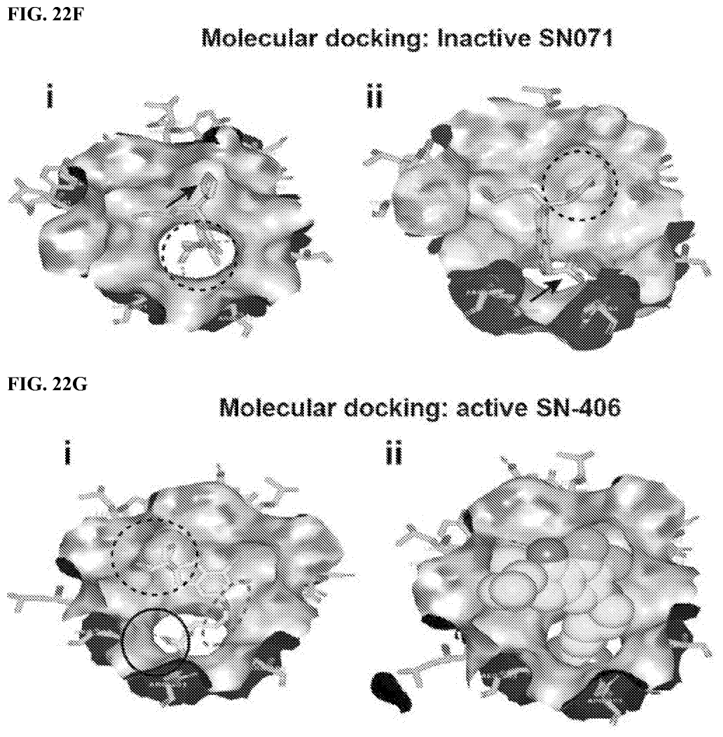

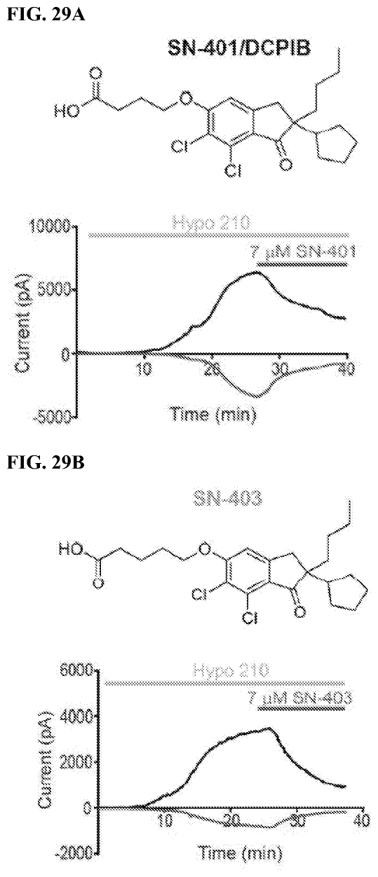

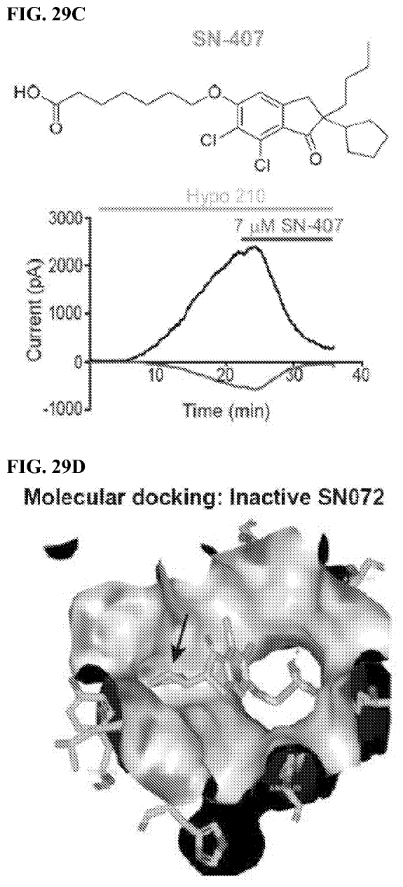

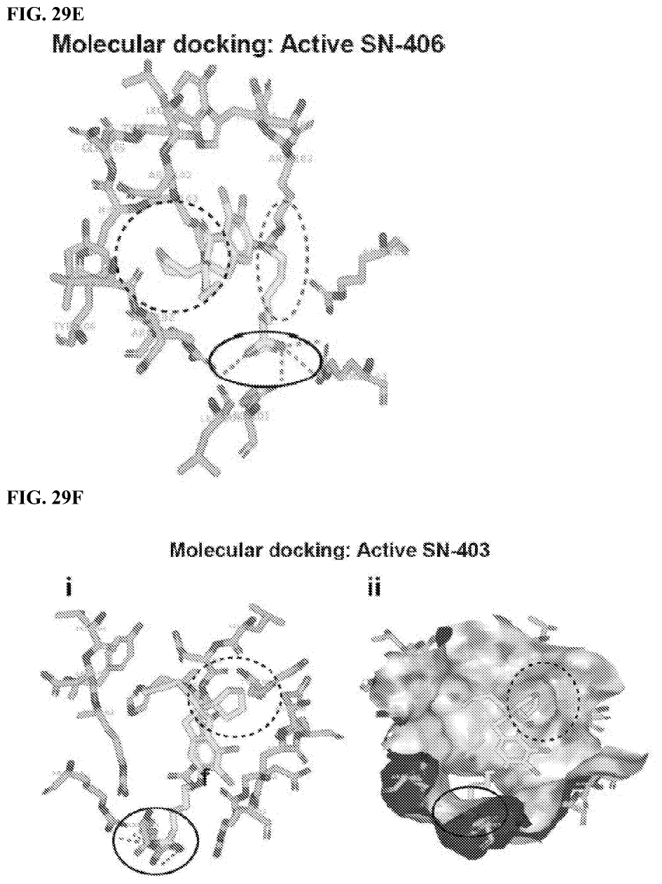

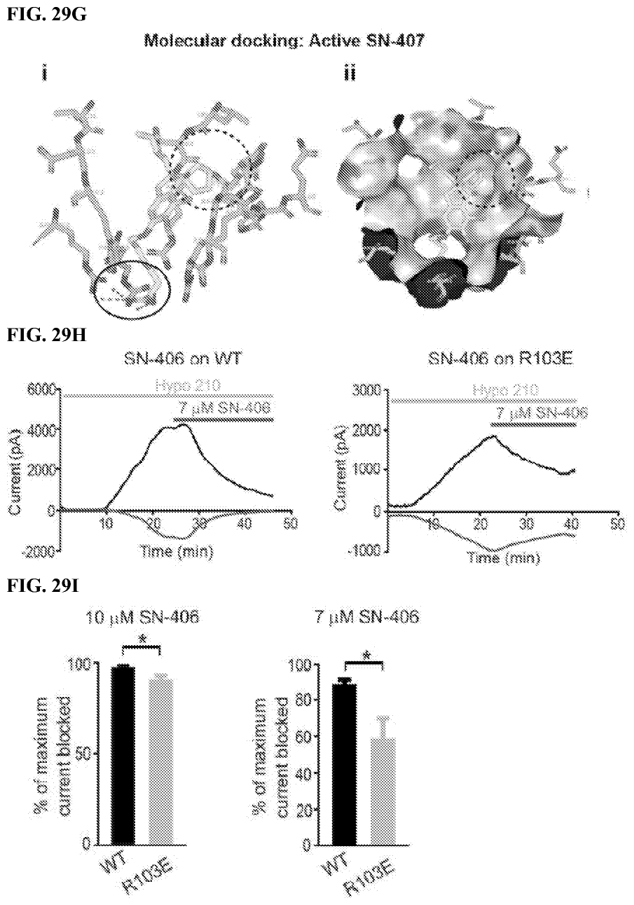

To confirm the SN-401-induced increases in SWELL1 protein were mediated by direct binding to the SWELL1-LRRC8 channel complex, as opposed to off-target effects, we designed and synthesized novel SN-401 congeners with subtle structural changes that either maintained or enhanced (SN-403, SN-406, SN-407; FIG. 22A), or entirely eliminated (S1\1071, SN072; FIG. 22A) SN-401 on-target inhibition of I_{Cl,SWELL} (FIGS. 22B and 22C; FIGS. 29A-29C). During the course of this work, Kern D. M. et. al. published a cryo-EM structure of SN-401/DCPIB bound with the SWELL1 homomer (Kern et al., 2019). This structure revealed that SN-401 binds at a constriction point in the SWELL1/LRRC8a homo-hexamer pore wherein the electronegative SN-401 carboxylate group interacts electrostatically with the R103 residue in one or more of the SWELL1 monomers (FIG. 22D). Moreover, SN-401 was required to obtain resolvable cryo-EM images in lipid-nanodiscs (Kern et al., 2019), as though stabilizing the SWELL1 hexamer.

To characterize the structural features of SN-401 responsible for binding to SWELL1-LRRC8, we performed molecular docking simulations of SN-401 and its analogs into the SWELL1 homo-hexamer (PDB: 6NZZ), and identified two molecular determinants predicted to be critical for SN-401-SWELL1-LRRC8 binding (FIG. 22E): (1) The length of the carbon chain leading to the anionic carboxylate group predicted to electrostatically interact with one or more R103 guanidine groups (found in SWELL1/LRRC8a and LRRC8b); and (2) Proper orientation of the hydrophobic cyclopentyl group that slides into a hydrophobic cleft at the interface of LRRC8 monomers (conserved among all LRRC8 subunit interfaces). Docking simulations predicted shortening the carbon chain leading to the carboxylate by 2 carbons would yield a molecule, SN071, that could interact with either R103 through the carboxylate group (FIG. 22F(,)), or have the cyclopentyl ring occupy the hydrophobic cleft (FIG. 22F(it)), but unable to participate in both interactions simultaneously (FIG. 22F, black arrows). Similarly, the SN-401 analog lacking the butyl group, SN072, is predicted to be unable to orient the cyclopentyl group into a position favorable for interaction with the hydrophobic cleft without introducing structural strain in the molecule (FIG. 29D, black arrow). Both of these structural modifications, predicted to abrogate either carboxylate-R103 electrostatic binding or cyclopentyl-hydrophobic pocket binding were sufficient to eliminate I_{Cl,SWELL} inhibitory activity in vitro (FIGS. 22B and 22C). Conversely, lengthening the carbon chain attached to the carboxylate group by 1-3 additional carbons resulted in compounds predicted to enhance R103 electrostatic interactions (FIG. 22G; FIGS. 29E-29G, black solid circle), and better orient the cyclopentyl group to bind within the hydrophobic cleft (FIG. 22G, FIG. 29E and FIG. 29F, black dash circle).

Additional binding interactions for congeners SN-406 and SN-407 are also predicted along the channel, due to the longer carbon chains affording additional hydrophobic interactions with side chain carbons of the R103 residues (FIG. 22G; FIG. 29E, gray dashes). This is anticipated to increase SN-406/SN-407 I_{Cl,SWELL} inhibitory activity and this is precisely what was observed (FIGS. 22B AND 22C; FIGS. 29A-29C). To further test this drug-channel binding model, we overexpressed an R103E mutant SWELL1 construct on a WT background, since the binding model predicts that reducing the electropositivity of the pore constriction by replacing the electropositive R103 with an electronegative glutamate residue (E103) will diminish SN-406 I_{C}1, SWELL inhibitory activity. Consistent with the prediction of this binding model, R103E expressing HEK cells exhibit reduced SN-406-mediated I_{Cl,SWELL} inhibition (FIGS. 29H and 29I).

Collectively, these functional and molecular docking experiments indicate SN-401 and SWELL1-active congeners (SN-403/406/407) bind to SWELL1-LRRC8 hexamers at both R103 (via carboxylate end) and at the interface between LRRC8 monomers (via hydrophobic end), to stabilize the closed state of the channel, thereby inhibiting I_{Cl,SWELL} activity. Guided by docking studies and binding models that reveal the SN-401 carboxylate group interacting with R103 residues of multiple LRRC8 monomers within the hexameric channel, along with SN-401 cyclopentyl group binding within hydrophobic clefts between adjacent monomers, we hypothesized that these SN-40X compounds function as molecular tethers to stabilize assembly of the SWELL1-LRRC8 hexamer. This reduces SWELL1-LRRC8 complex disassembly, and subsequent proteasomal degradation, thereby augmenting translocation from ER to plasma membrane signaling domains, functioning as a pharmacological chaperone.

### Example 9: SN-401 and SWELL1-Active Congener SN-406 Function as Pharmacological Chaperones at Sub-Micromolar Concentrations

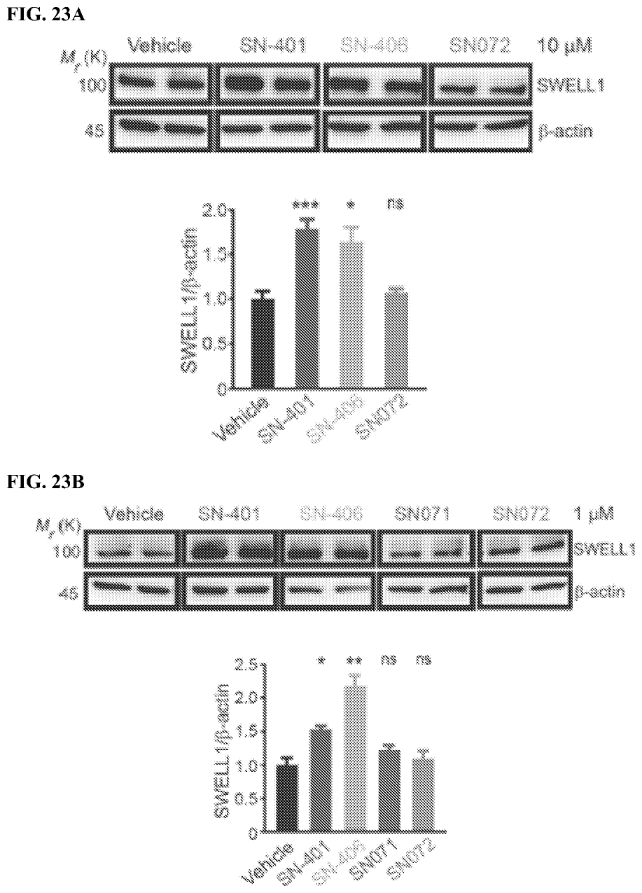

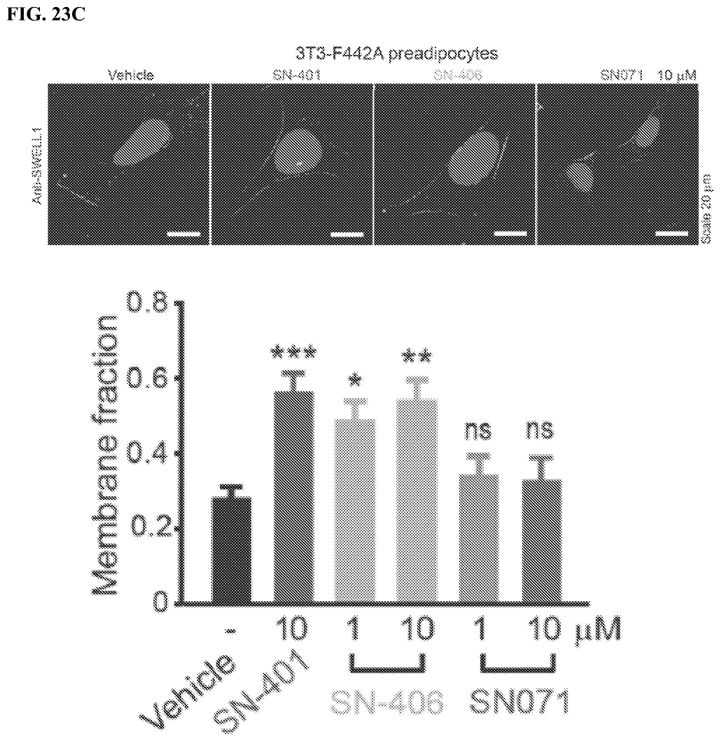

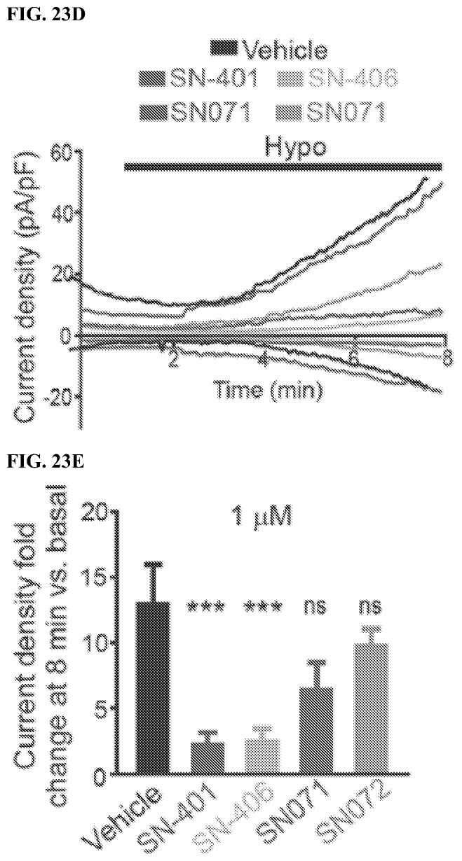

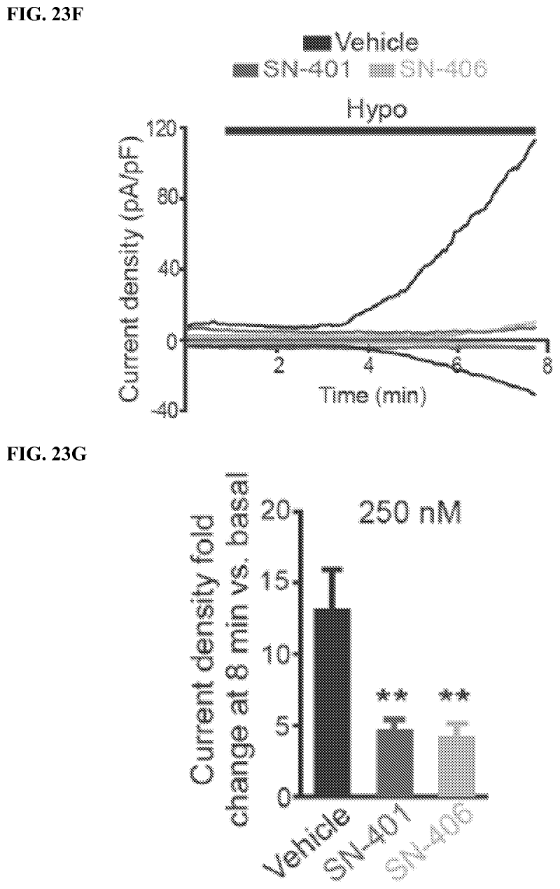

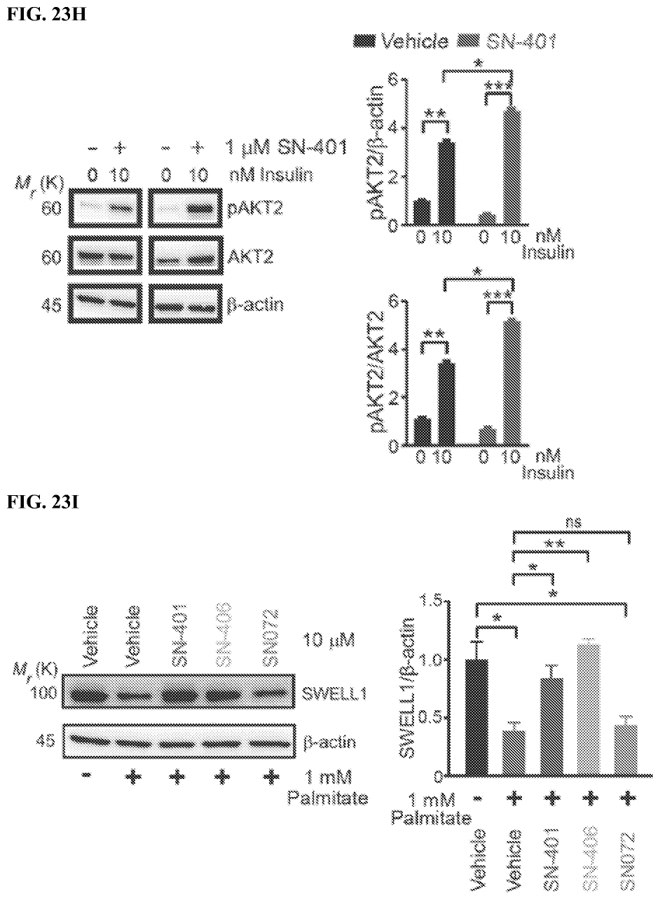

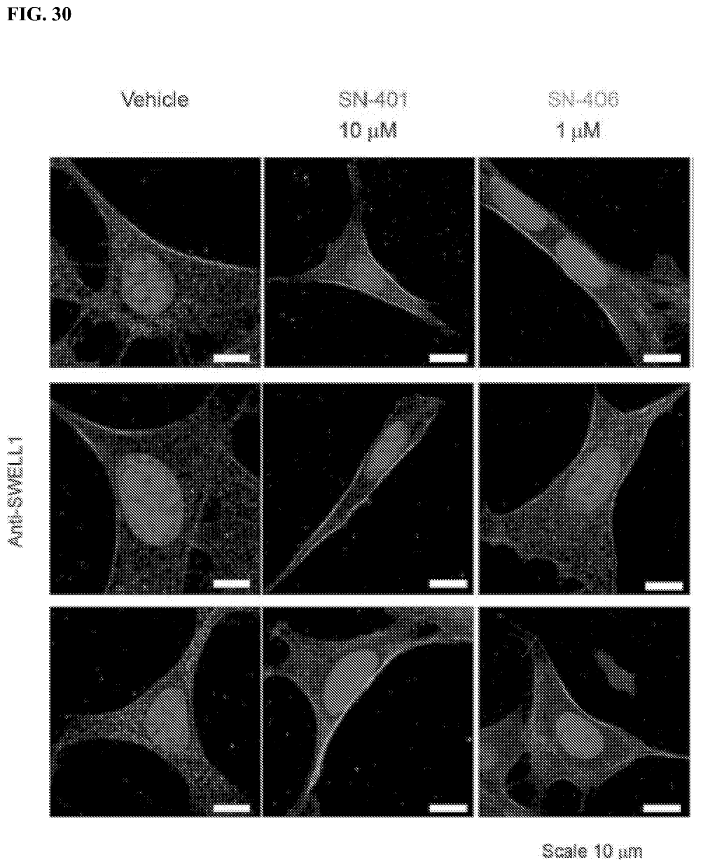

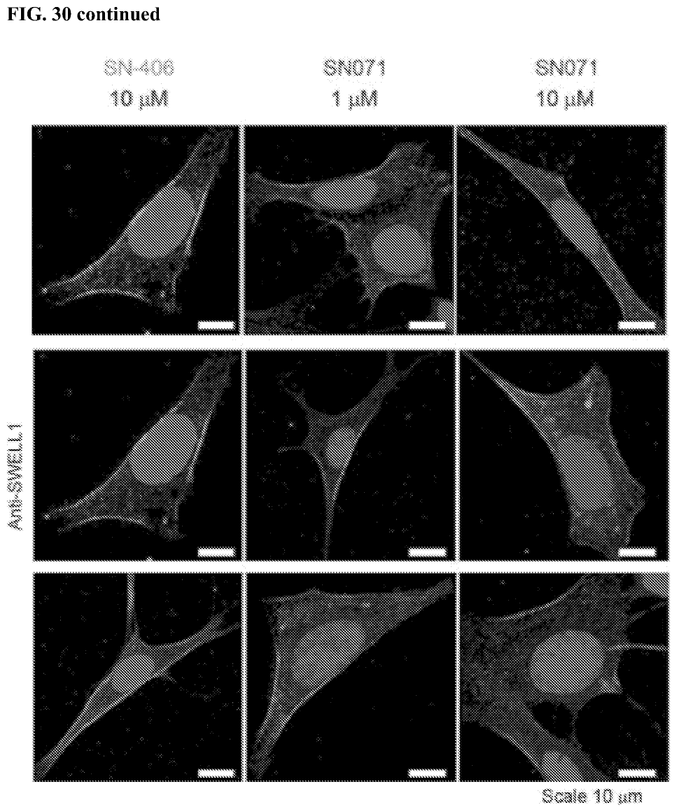

To test this hypothesis, we applied SWELL1-active SN-401 and SN-406 compounds to differentiated 3T3-F442A adipocytes under basal culture conditions for 4 days and then measured SWELL1 protein after 6 h of serum starving. At both 1 and 10 μM, SN-401 and SN-406 markedly augment SWELL1 protein to levels 1.5-2.3-fold to those in vehicle-treated controls, while inactive congeners SN071 and SN072 do not significantly increase SWELL1 protein levels. (FIGS. 23A and 23B). SN-401 and SN-406 also enhanced plasma membrane (PM) localization of endogenous SWELL1 in preadipocytes compared to vehicle- or SN071 (FIG. 23C, FIG. 30), consistent with increased endoplasmic reticulum (ER) to plasma membrane trafficking of SWELL1, and pharmacological chaperone activity. Notably, SN-401 and SN-406 are capable of augmenting both SWELL1 protein and trafficking at concentrations as low as 1 μM showing the EC₅₀ for SN-401 and SWELL1-active congeners binding to SWELL1-LRRC8 in the closed or resting state is <1 μM, or an order of magnitude below the ˜10 μM concentration required for inhibiting activated SWELL1-LRRC8 (upon hypotonic stimulation). Indeed, application of SN-401 or SN-406 to HEK cells for 30 minutes prior to hypotonic activation at both 1 μM (FIGS. 23D and 23E) and 250 nM (FIGS. 23F and 23G) markedly suppresses and delays subsequent hypotonic SWELL1-LRRC8 activation, in contrast to either vehicle or to inactive SN071 and SN072 compounds (FIGS. 23D and 23E). These data support the notion that SN-40X compounds bind with higher affinity to SWELL1-LRRC8 channels in the closed state than the open state, and putatively stabilize the closed conformation of the channel to inhibit I_{Cl,SWELL}. Moreover, these data indicate SN-401 and its SWELL1-active congeners, SN-40X, function as pharmacological chaperones at less than one-tenth the concentration required to inhibit activated SWELL1-LRRC8 channels. Indeed, treating 3T3-F442A adipocytes with 1 μM SN-401 for 96 hours, followed by washout, also robustly increases insulin-pAKT2 signaling compared to vehicle (FIG. 23H).

We next asked whether endoplasmic reticulum (ER) stress associated with glucolipotoxicity in metabolic syndrome may impair SWELL1-LRRC8 assembly and trafficking, to promote SWELL1 protein degradation, and thereby reduce I_{Cl,SWELL} and SWELL1 protein in T2D (FIGS. 20A-20F). In this context, we hypothesized that pharmacological chaperones (SN-401-406) might assist with SWELL1-LRRC8 assembly and rescue SWELL1-LRRC8 from degradation. To test this concept in vitro, we first treated 3T3-F442A adipocytes with either vehicle, SN-401, SN-406 or SN072, and then subjected these cells to 1 mM palmitate+25 mM glucose to induce to glucolipotoxic stress (FIG. 23I). We found that SWELL1 protein was reduced by 50% upon palmitate/glucose treatment, consistent with ER stress-mediated SWELL1 degradation, and this reduction was entirely prevented by both SWELL1-active SN-401 and SN-406, but not by SWELL1-inactive SN072 (FIG. 23I). These data are consistent with the notion that SN-401 and SWELL1-active congeners are functioning as pharmacological chaperones to stabilize SWELL1-LRRC8 assembly and signaling under glucolipotoxic conditions associated with T2D and metabolic syndrome.

### Example 10: SN-401 Increases SWELL1 and Improves Systemic Glucose Homeostasis in Murine T2D Models by Enhancing Insulin Sensitivity and Secretion

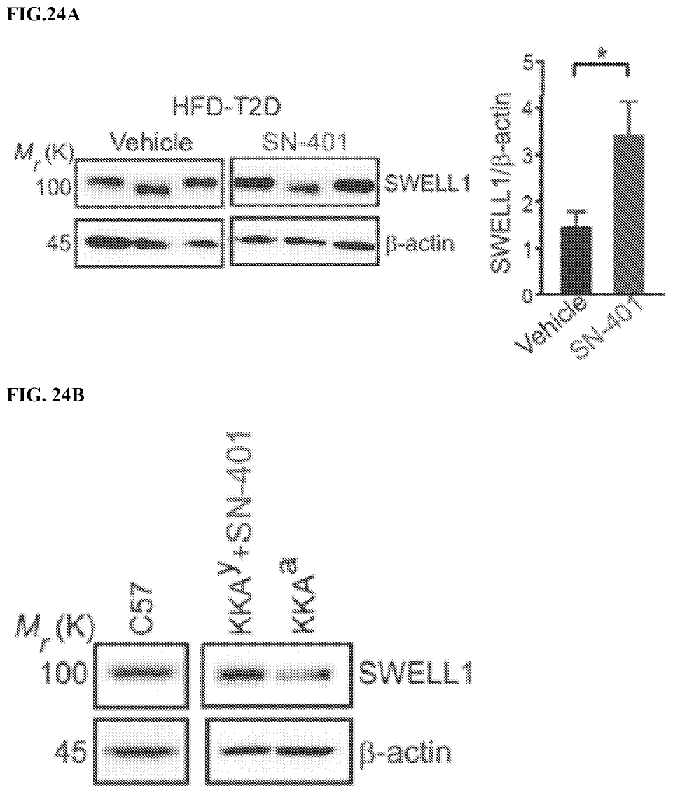

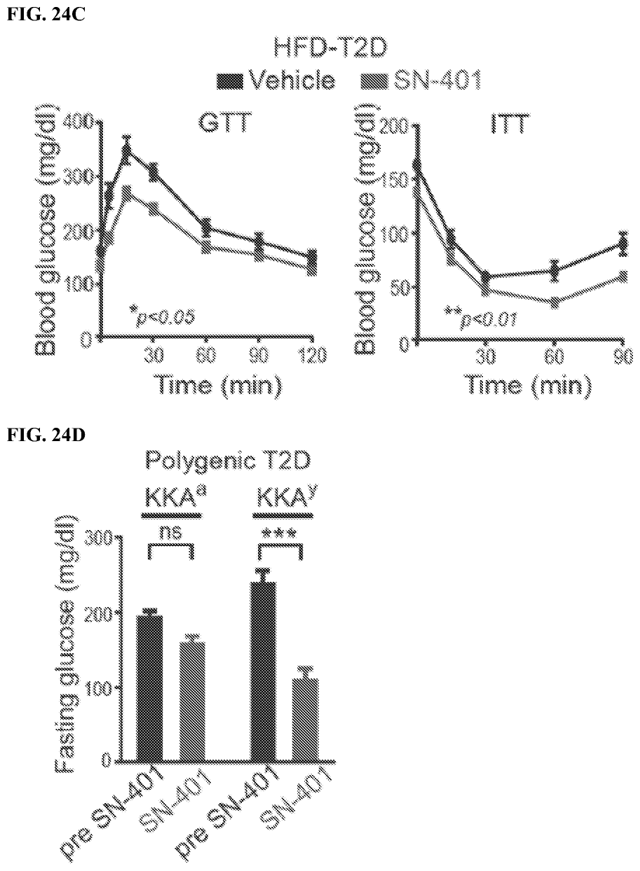

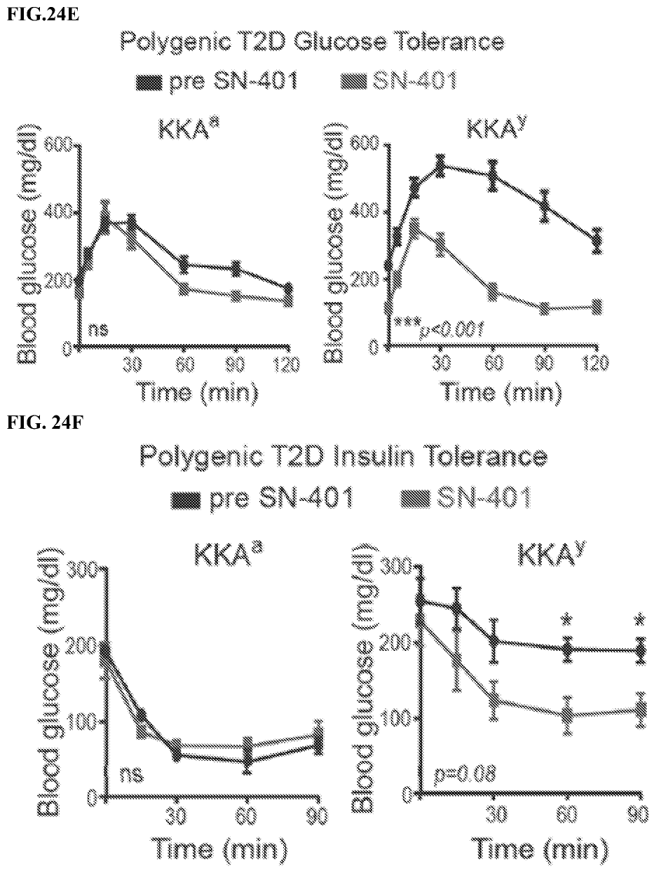

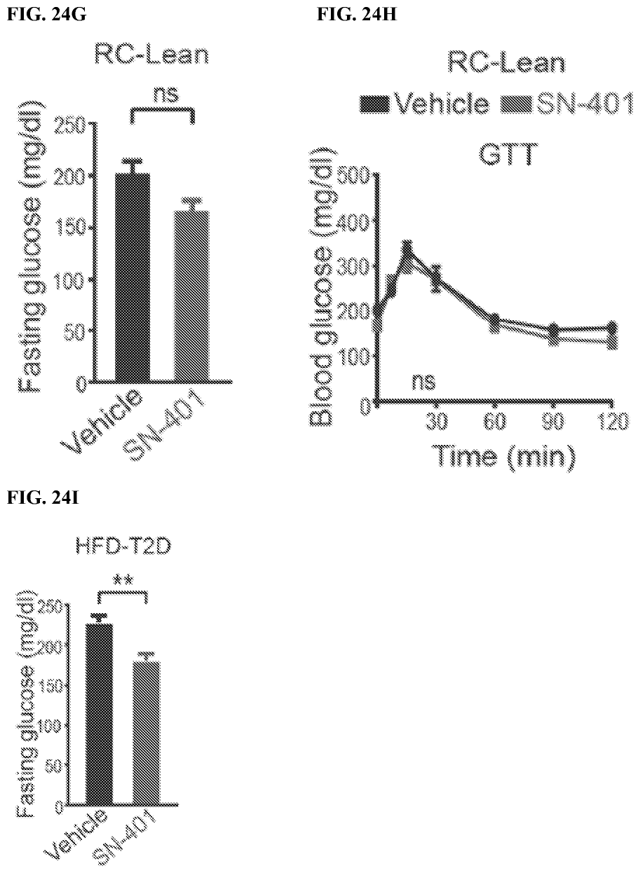

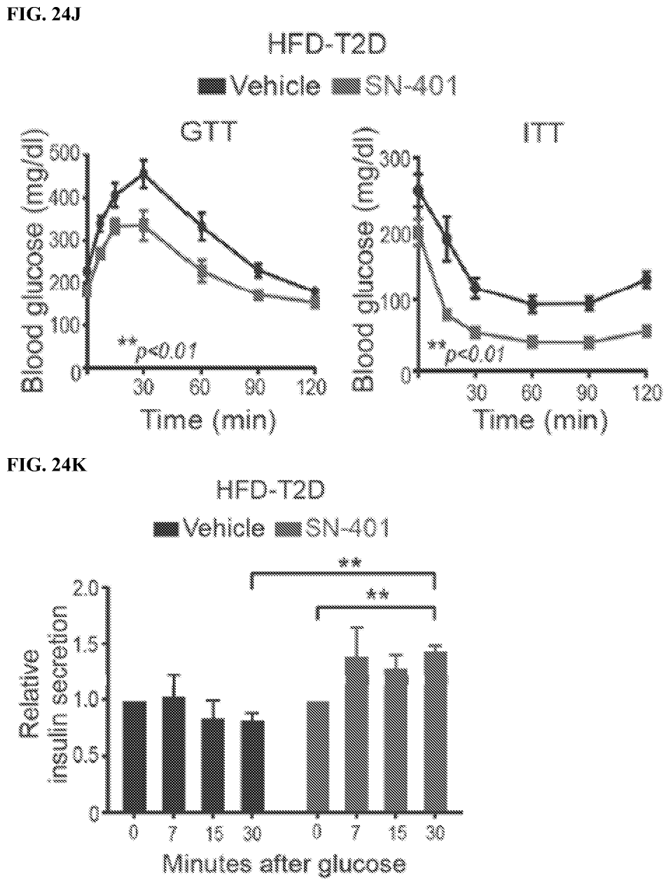

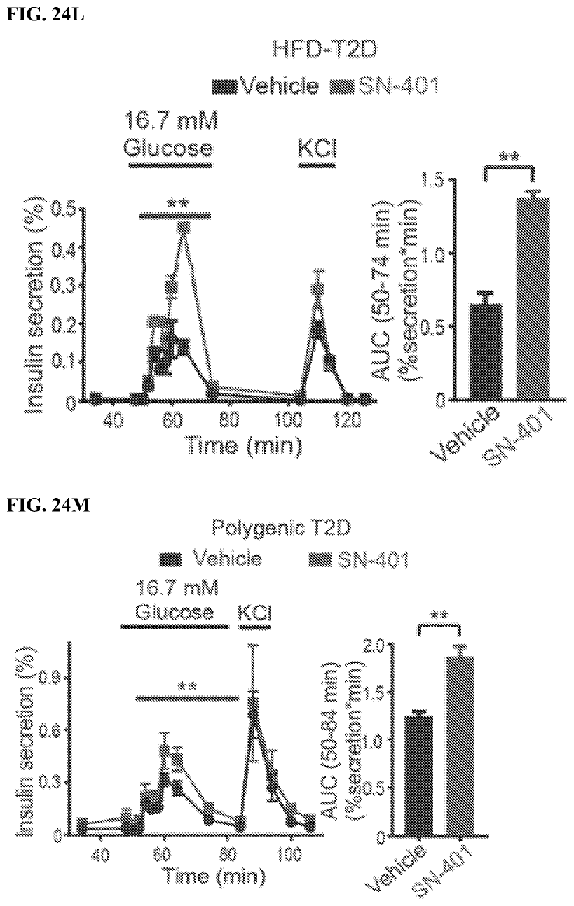

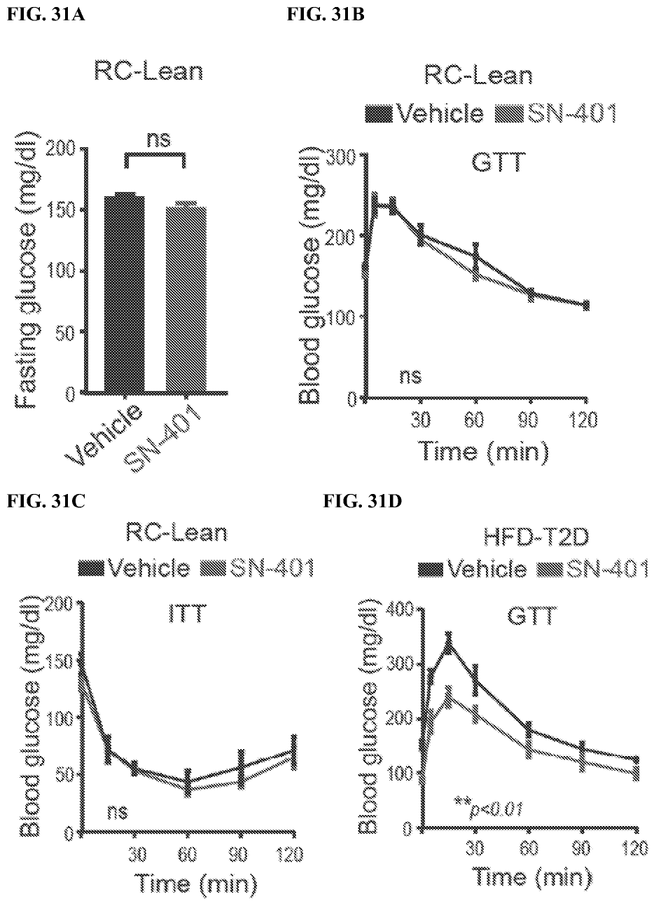

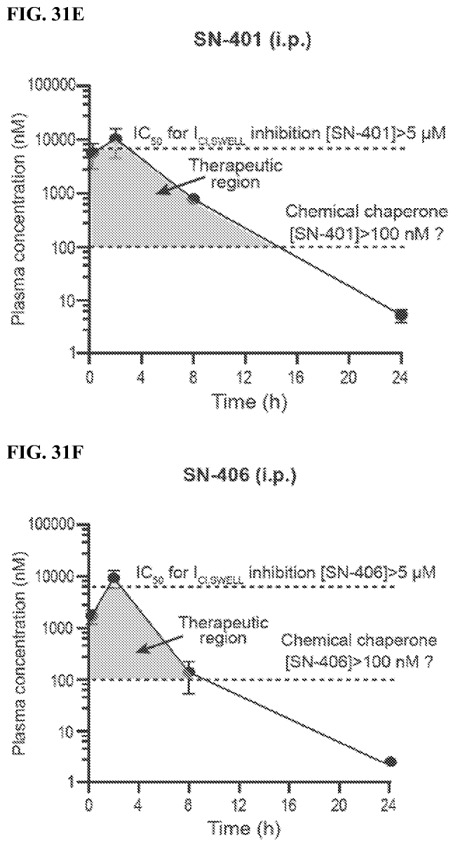

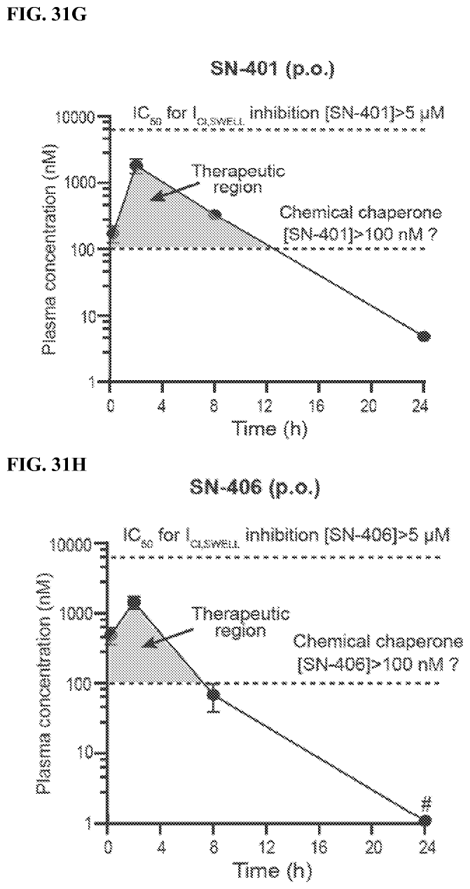

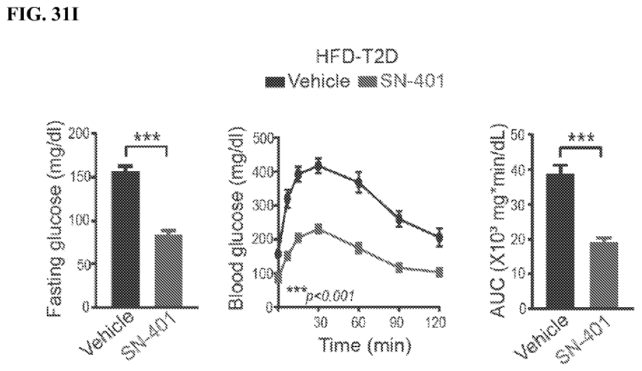

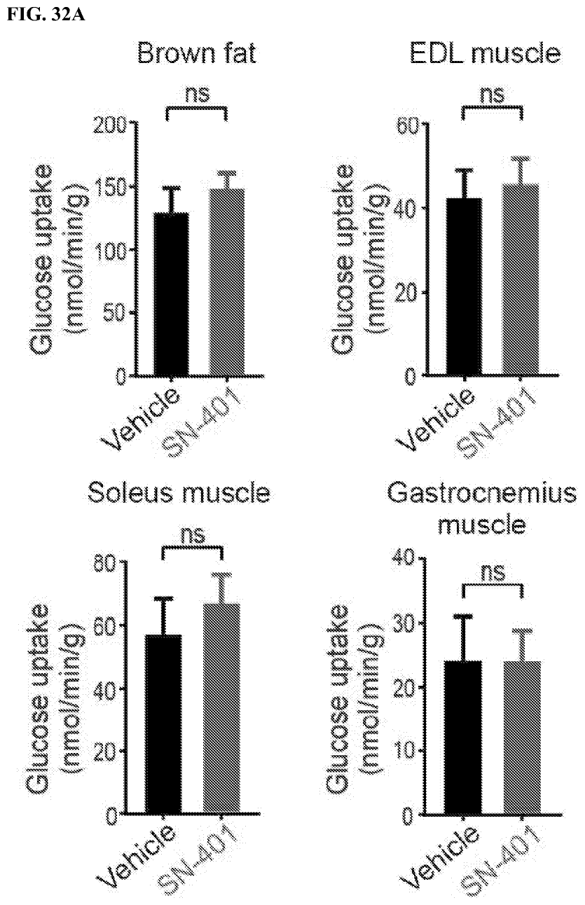

To determine if SN-401 improves insulin signaling and glucose homeostasis in vivo we treated two T2D mouse models: obese, HFD-fed mice and the polygenic T2D KKN mouse model with SN-401 (5 mg/kg i.p. for 4-10 days). In vivo, SN-401 augments SWELL1 expression 2.3-fold in adipose tissue of HFD-fed T2D mice (FIG. 24A). Similarly, SN-401 increases SWELL1 expression in adipose tissue of T2D KKN mice to levels comparable to both non-T2D C57/B6 mice and to the parental KKAa parental strain (FIG. 24B). This restoration of SWELL1 expression is associated with normalized fasting blood glucose (FG), glucose tolerance (GTT), and markedly improved insulin-tolerance (ITT) in both HFD-induced T2D mice (FIG. 24C) and in the polygenic T2D KKAy model (FIGS. 24D-24F). Remarkably, treating the control KKAa parental strain with SN-401 at the same treatment dose (5 mg/kg×4-10 days) does not cause hypoglycemia, nor does it alter glucose and insulin tolerance (FIGS. 24D-24F). Similarly, lean, non-T2D, glucose-tolerant mice treated with SN-401 have similar FG, GTT and ITT compared to vehicle-treated mice (FIGS. 24G and 24H and FIGS. 31A-31C). However, when made insulin-resistant and diabetic after 16 weeks of HFD feeding, these same mice (from FIGS. 24G and 24H) treated with SN-401 show marked improvements in FG (FIG. 24I), GTT and ITT (FIG. 24J) as compared to vehicle. These data show that SN-401 restores glucose homeostasis in the setting of T2D, but has little effect on glucose homeostasis in non-T2D mice. Importantly, this portends a low risk for inducing hypoglycemia. SN-401 was well-tolerated during chronic i.p. injection protocols, with no overt signs of toxicity with daily i.p. injections for up to 8 weeks, despite striking effects on glucose tolerance (FIG. 31D). In fact, in vivo pharmacokinetics (PK) of SN-401 and SN-406 in mice following i.p. or p.o. administration of 5 mg/kg of SN-401 or SN-406 reveal plasma concentrations that either transiently approach (FIGS. 31E and 31F, i.p. dosing), or remain well below I_{Cl,SWELL} inhibitory concentrations (FIGS. 31G and 31H, p.o. dosing) while exceeding concentrations sufficient for SWELL1 pharmacological chaperone activity {>˜100 nM) for 8-12 hours.

SN-401 has in silica, in vitro, and in vivo characteristics that show it may be an effective oral therapy for T2D. First, several algorithms designed to identify candidate compounds with oral drug-like physicochemical properties (Lipinski (Lipinski et al., 2001), Veber (Veber et al., 2002), Egan (Egan et al., 2000), MDDR (Oprea, 2000)) indicate that SN-401 had oral drug-like properties as compared to current approved oral T2D drugs (Table 7, below).

TABLE 7
In silico predicted drug likeness of SN-401
and SN-406 are similar to common T2D drugs

| | Compound/Approved Drug | | | | |
|---|---|---|---|---|---|
| Predicted Property | SN-401 | SN-406 | Metformin | Empagliflozin | Software |
| Physiochemical | | | | | |
| MW (g/ml) | 420-430 | 455.4 | 129.2 | 450.9 | FAF- |
| | | | | | Drugs4 |
| Buffer solubility (mg/L) | 1222.3 | 315.5 | 18299.7 | 148.7 | preADMET |
| ADMET | | | | | |
| In vitro hERG inhibition | Low | Low | Medium | Medium risk | preADMET |
| | risk | risk | risk | | |
| Drug Likeness | | | | | |
| Lipinski's rule | Suitable | Suitable | Suitable | Suitable | preADMET |
| (Rule of five) | | | | | |
| Veber rule | Good | Good | Good | Good | FAF- |
| | | | | | Drugs4 |
| MDDR-like rule: Nondrug- | Drug- like | Drug- like | Drug-like | Drug-like | preADMET |
| like/drug-like/mid | | | | | |
| Egan Rule | Good | Good | Good | Good | FAF- |
| | | | | | Drugs4 |

Second, in vitro studies show SN-401 has good Caco-2 cell monolayer permeability and minimal cytochrome p450 isoenzyme inhibition (Table 8, below). Third, SN-401 has no effect on hERG, human Kv and delayed rectifier channels, and is selective for I_{Cl,SWELL} in guinea-pig atrial cells at channel inhibitory concentrations {˜5-10 μM), which is consistent with in silica ADMET predictions (Table 7), and indicates a low likelihood of cardiac QT prolongation and arrhythmia Fourth, in vivo PK studies in mice demonstrate that SN-401 has high oral bioavailability (AUCp.o./AUCi.v.=79%, FIGS. 31G and 31H, and Table 9, below), and SN-401 administered via oral gavage to HFD-fed T2D C57 mice at 5 mg/kg/day fully retains in vivo therapeutic efficacy (FIG. 31I).

TABLE 8
In vitro absorption, metabolism, and CYP450
isoenzyme inhibition of SN-401 and SN-406

Compound

| In vitro property | SN-401 | SN-406 |
|---|---|---|
| Caco-2 10⁻⁶ cm/s | 8.24 | 1.93 |
| Caco-2 permeability ranking | Higher | Higher |
| Caco-2 efflux ratio B-A/A-B | 1.44 | 1.58 |
| Caco-2 efflux ranking | Not | Not |
| | significant | significant |
| Human hepatic microsome stability | 72.7 | 46.4 |
| Cl_{intrinsic} mL/min/kg | | |
| CYP 2C9 inhibition IC₅₀, μM | <10 | >10 |
| CYP 2D6 inhibition IC₅₀, μM | >10 | >10 |
| CYP 3A4 inhibition IC₅₀, μM | >10 | >10 |

TABLE 9
SN-401 and SN-406 in vivo PK parameters

| | SN-401 | | | SN-406 | | |
|---|---|---|---|---|---|---|
| PK Parameters | Oral | Intravenous | Intraperitoneal | Oral | Intravenous | Intraperitoneal |
| AUCinf | 4682 | 5958 | 23030 | 3131 | 6532 | 18180 |
| (ng*h/mL) | | | | | | |
| Oral | 79% | NA | NA | 48% | NA | NA |
| bioavailability | | | | | | |
| Cmax (ng/mL) | 781 | 5443 | 4367 | 660.7 | 15130 | 4300 |
| T-half (h) | 2.585 | 1.428 | 2.056 | 2.058 | 0.7689 | 1.809 |

To examine the possible contribution of SN-401-mediated enhancements in insulin secretion from pancreatic β-cells, we next measured glucose-stimulated insulin secretion (GSIS) in SN-401 treated mice subjected to 21 weeks of HFD. We find that the impairments GSIS classically observed with long-term HFD (21 weeks HFD) are significantly improved in SN-401-treated HFD mice based on serum insulin measurements (FIG. 24K) and perifusion GSIS from isolated islets (FIG. 24L), consistent with the predicted effect of SWELL1 induction in pancreatic β-cells. Similar results are obtained in perfusion assays performed in SN-401 compared to vehicle treated T2D KKN mice (FIG. 24M). Collectively, these data show that SN-401-mediated improvements in systemic glycemia in T2D occur via augmentation of both peripheral insulin sensitivity and β-cell insulin secretion via SN-401 pharmacological chaperone mediated SWELL1-LRRC8 gain-of-function—the inverse phenotype to in vivo loss-of-function studies (Kang et al., 2018 and Zhang et al., 2017).

### Example 11: SN-401 Improves Systemic Insulin Sensitivity, Tissue Glucose Uptake, and Nonalcoholic Fatty Liver Disease in Murine T2D Models

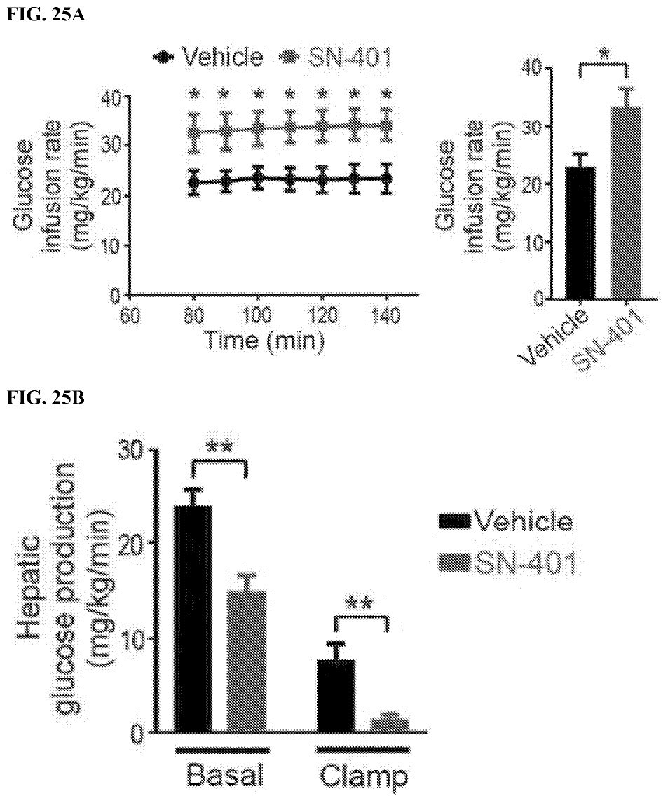

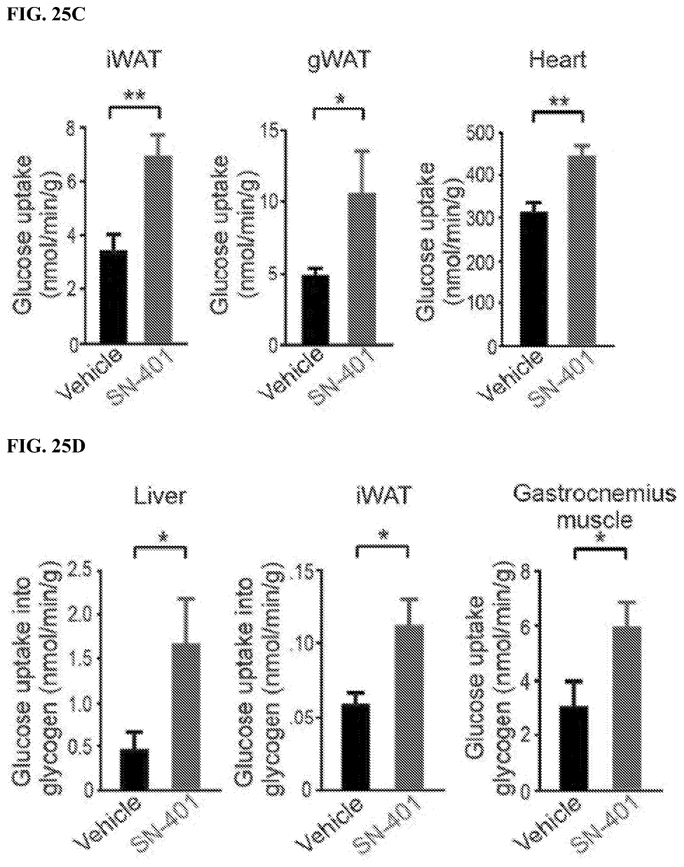

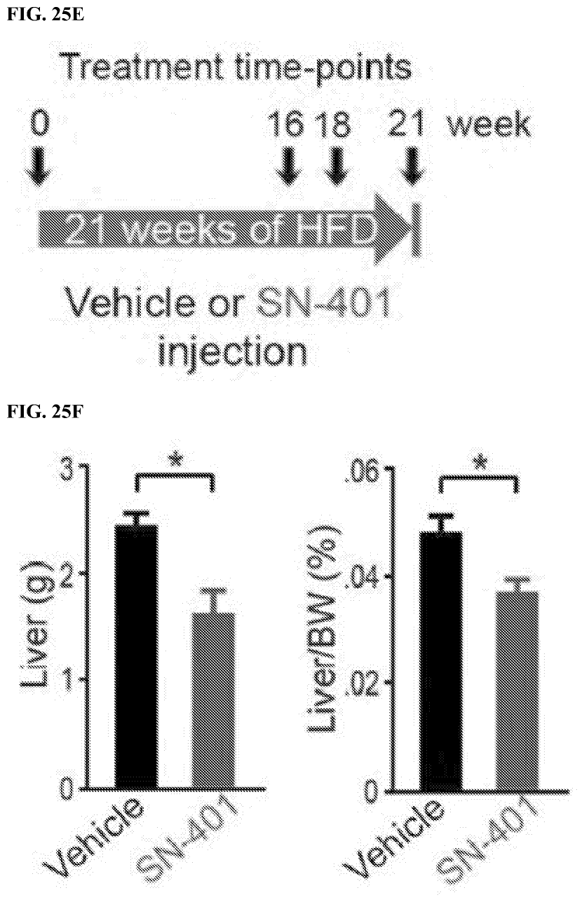

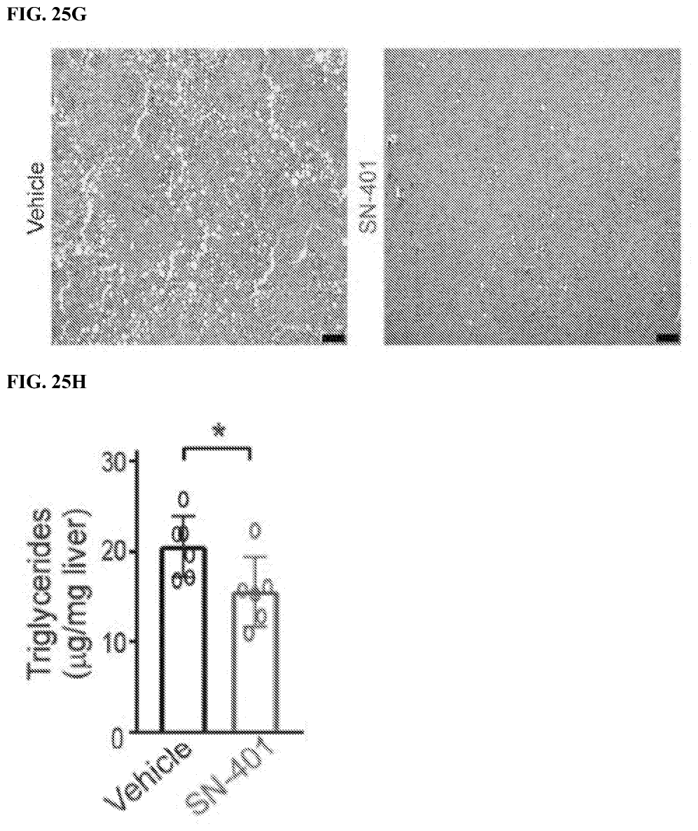

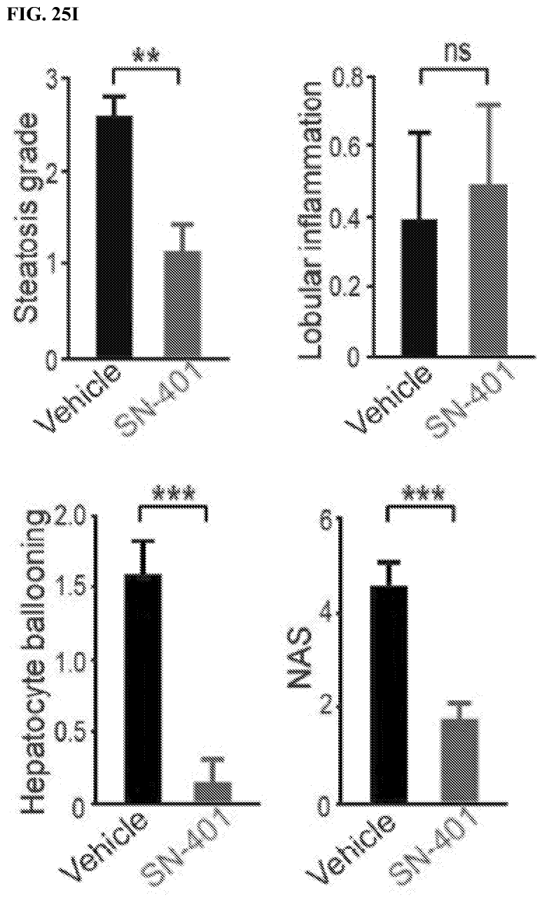

To more rigorously evaluate SN-401 effects on insulin sensitization and glucose metabolism in T2D mice we compared euglycemic hyperinsulinemic clamps traced with 3H-glucose and 14C-deoxyglucose in T2D KKN mice treated with SN-401 or vehicle. SN-401 treated T2D KKN mice require a higher glucose-infusion rate (GIR) to maintain euglycemia compared to vehicle, consistent with enhanced systemic insulin-sensitivity (FIG. 25A). Hepatic glucose production from gluconeogenesis and/or glycogenolysis (Ra, rate of glucose appearance) is reduced 40% in SN-401-treated T2D KKN mice at baseline (Basal, FIG. 25B), and further suppressed 75% during glucose/insulin infusion (Clamp, FIG. 25B). These data demonstrate SN-401 increases hepatic insulin sensitivity.

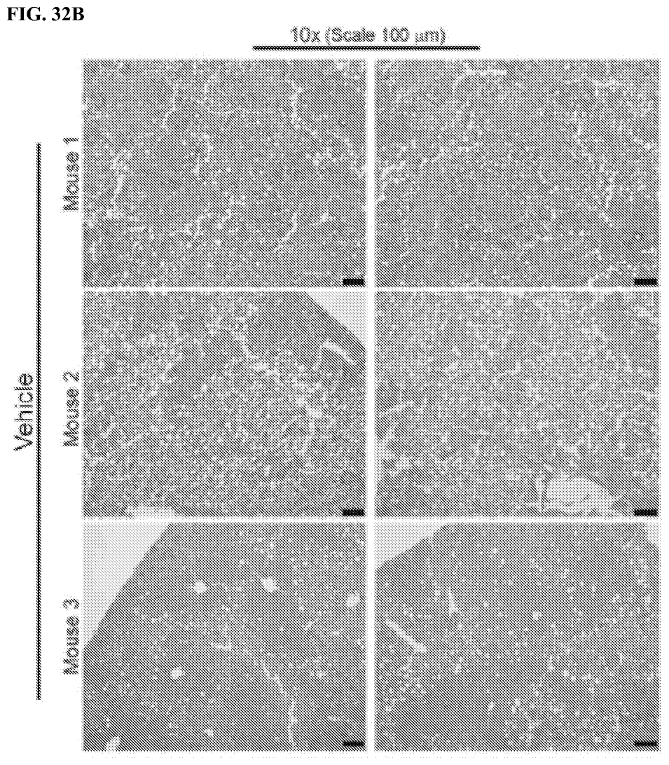

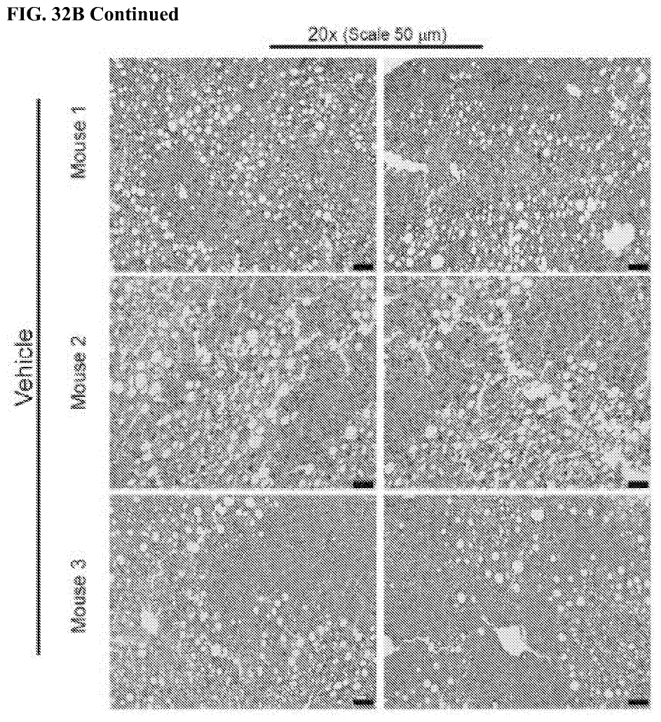

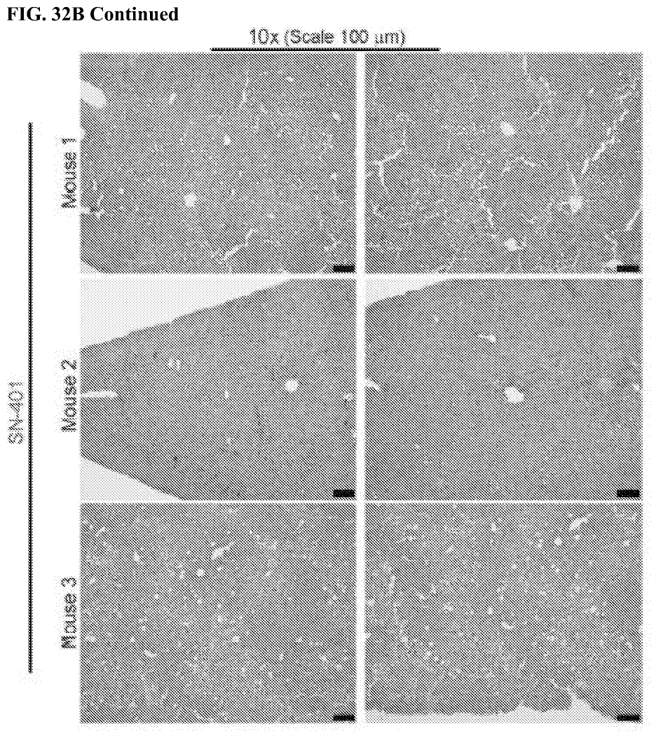

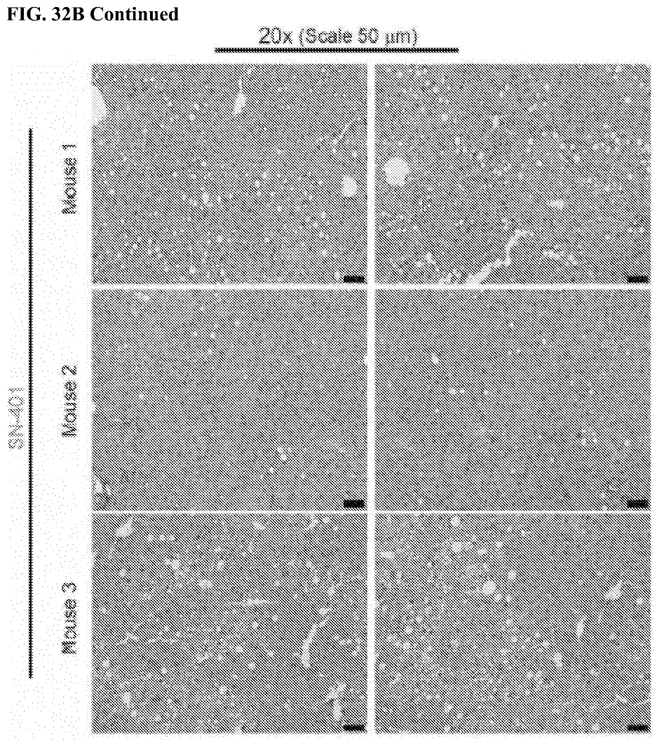

As the SN-401-mediated increase in SWELL1 is expected to enhance insulin-pAKT2-pAS160 signaling, GLUT4 plasma membrane translocation, and tissue glucose uptake, we next measured the effect of SN-401 on glucose uptake in adipose, myocardium and skeletal muscle using 2-deoxyglucose (2-DG). SN-401 enhanced insulin-stimulated 2-DG uptake into inguinal white adipose tissue (iWAT), gonadal white adipose tissue (gWAT), and myocardium (FIG. 25C), but not in brown fat or skeletal muscle (FIG. 32A). As adipocyte SWELL1 ablation markedly reduces insulin-pAKT2-pGSK3-regulated cellular glycogen content we next asked whether the SN-401-mediated increase in SWELL1 would increase glucose incorporation into tissue glycogen in the setting of T2D. Indeed, liver, adipose, and skeletal muscle glucose incorporation into glycogen is markedly increased in SN-401-treated mice (FIG. 25D), consistent with a SWELL1-mediated insulin-pAKT2-pGSK3-glycogen synthase gain-of-function.

Nonalcoholic fatty liver disease (NAFLD), like T2D, is associated with insulin resistance. NASH is an advanced form of nonalcoholic liver disease defined by three histological features: hepatic steatosis, hepatic lobular inflammation, hepatocyte damage (ballooning) and can be present without or without fibrosis. NAFLD and T2D likely share at least some pathophysiologic mechanisms because more than one-third of patients (37%) with T2D have NASH and almost one-half of patients with NASH (44%) have T2D. (To evaluate the effect of SN-401 on the genesis of NAFLD, mice were raised on HFD for 16 weeks followed by intermittent dosing with SN-401 over the course of 5 weeks (FIG. 25E). Mice treated with SN-401 had grossly smaller livers with reduced absolute and body mass-normalized liver mass, compared to vehicle-treated mice (FIG. 25F), and lower hepatic triglyceride concentration (FIG. 25H). Histologic evaluation showed mice treated with SN-401 had significantly reduced hepatic steatosis and hepatocyte damage compared to vehicle-treated mice (FIGS. 25F and 25J). In mice treated with SN-401 the NAFLD activity score (NAS), which integrates histologic scoring of hepatic steatosis, lobular inflammation, and hepatocyte ballooning (Kleiner et al., 2005) (FIG. 25I), also improved >2 points in SN-401-treated mice compared to vehicle-treated mice. Taken together, these data reveal SN-401 augments SWELL1 protein and SWELL1-mediated signaling to concomitantly enhance both systemic insulin sensitivity and pancreatic β-cell insulin secretion, thereby normalizing systemic glycemia in T2D mouse models. This improved metabolic state can reduce ectopic lipid deposition and NAFLD that is associated with obesity and T2D.

### Example 12: SWELL1-Active SN-401 Congeners Improve Systemic Glucose Homeostasis in Murine T2D

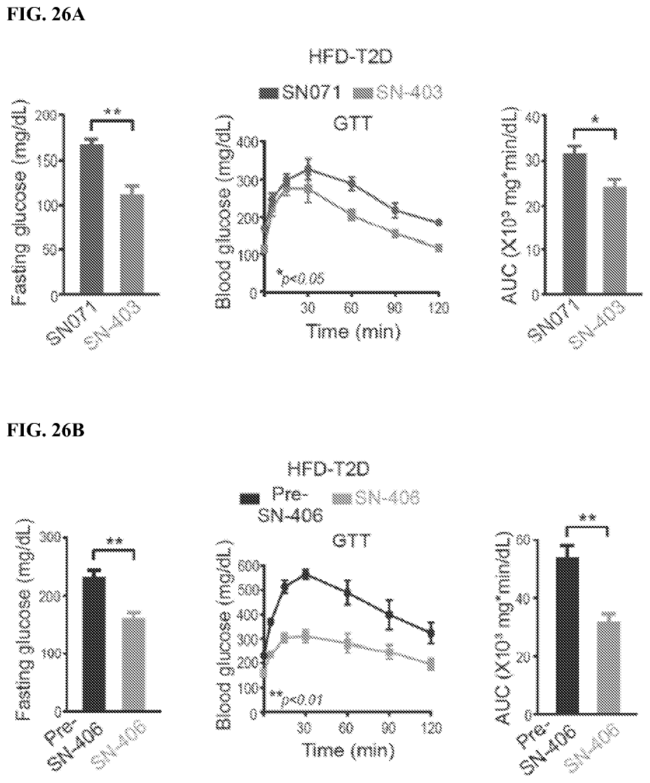

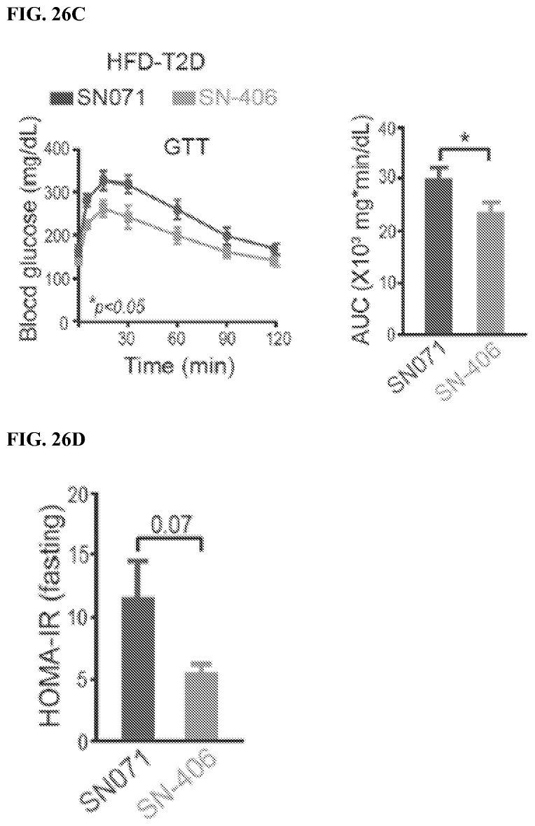

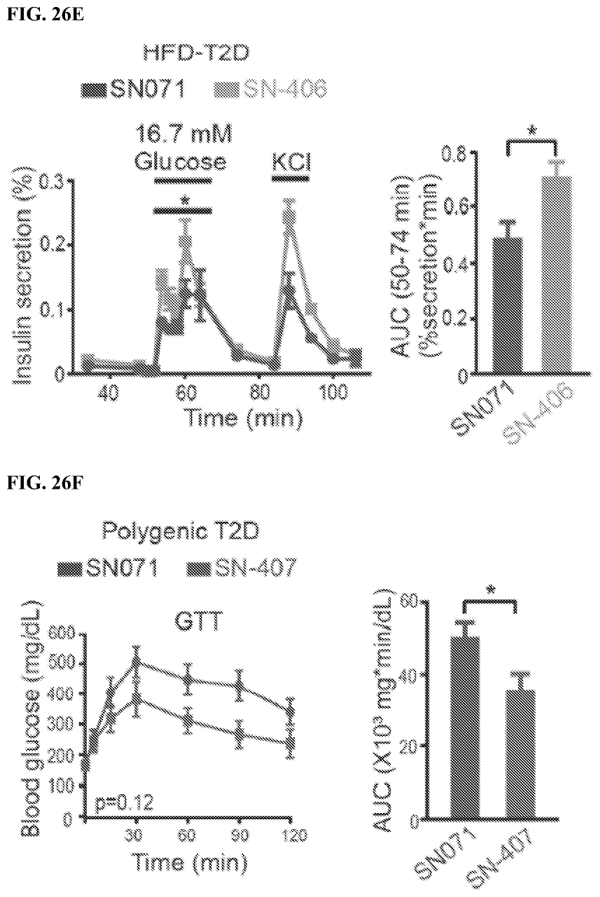

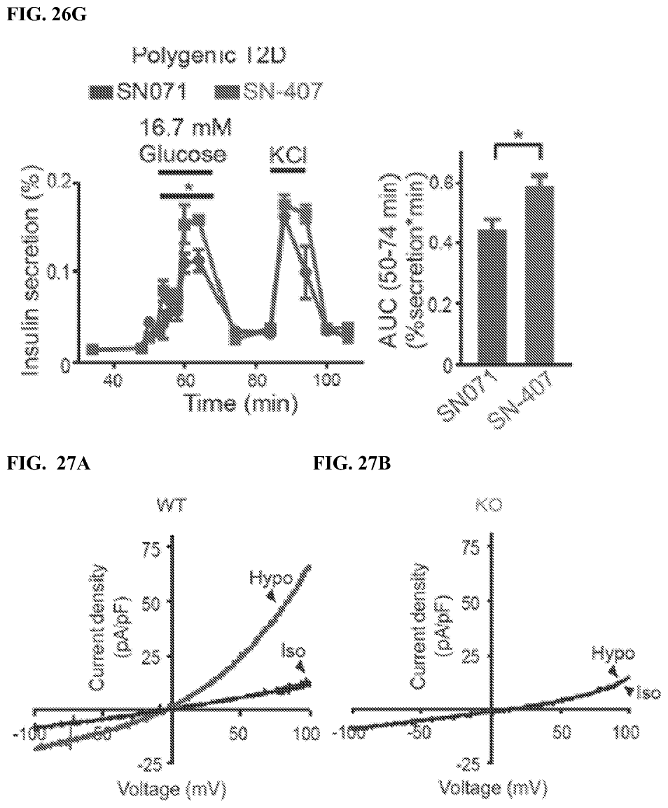

To determine if the effects of SN-401 observed in vivo in T2D mice are attributable to SWELL1-LRRC8 binding, as opposed to off-target effects, we next measured fasting blood glucose and glucose tolerance in HFD T2D mice treated with either SWELL1-active SN-403 or SN-406 as compared to SWELL1-inactive SN071 (all at 5 mg/kg/day×4 days). In mice treated with HFD for 8 weeks, SN-403 significantly reduced fasting blood glucose and improved glucose tolerance compared to SN071 (FIG. 26A). In cohorts of mice raised on HFD for 12-18 weeks, with more severe obesity-induced T2D, SN-406 also markedly reduced fasting blood glucose and improved glucose tolerance (FIG. 26B). Similarly, in a separate experiment, SN-406 significantly improved glucose tolerance in HFD T2D mice, compared to SWELL1-inactive SN071 (FIG. 26C), and this is associated with a trend toward improved insulin sensitivity based on the Homeostatic Model Assessment of Insulin Resistance (HOMA-IR) (Matthews et al., 1985) (FIG. 26D), and significantly augmented insulin secretion in perifusion GSIS (FIG. 26E). Finally, based on the GTT AUC, SN-407 also improved glucose tolerance in T2D KKN mice, compared to SN071 (FIG. 26F) and increased GSIS (FIG. 26G). These data reveal the in vivo anti-hyperglycemic action of SN-401 and its bioactive congeners require SWELL1-LRRC8 binding and thus supports the notion of SWELL1 on-target activity in vivo.

### Example 13: Discussion of Examples 6 to 12

Our current working model is that the transition from compensated obesity (pre-diabetes, normoglycemia) to decompensated obesity (T2D, hyperglycemia) reflects, among other things, a relative reduction in SWELL1 protein expression and signaling in peripheral insulin-sensitive tissues) and in pancreatic β-cells)-metabolically pheno-copying SWELL1-loss-of-function models. This contributes to the combined insulin resistance and impaired insulin-secretion associated with poorly-controlled T2D and hyperglycemia. SWELL1 forms a macromolecular signaling complex that includes heterohexamers of SWELL1 and LRRC8b-e, with stoichiometries that likely vary from tissue to tissue. We propose that SWELL1-LRRC8 signaling complexes are inherently unstable, and thus a proportion of complexes succumb to disassembly and degradation. Glucolipotoxicity and ensuing ER stress associated with T2D states provide an unfavorable environment for SWELL1-LRRC8 complex assembly, contributing to SWELL1 degradation and reductions in SWELL1 protein and SWELL1-mediated I_{Cl,SWELL} observed in T2D. Small molecules SN-401 and SN-401 congeners with preserved SWELL1 binding activity serve as pharmacological chaperones to stabilize formation of the SWELL1-LRRC8 complex. This reduces SWELL1 degradation, and enhances the passage of SWELL1-LRRC8 heteromers through the ER and Golgi apparatus to the plasma membrane—thereby rectifying the SWELL1-deficient state in multiple metabolically important tissues in the setting of T2D and metabolic syndrome to improve overall systemic glycemia via both insulin sensitization and secretion mechanisms. Indeed, the concept of small molecule inhibitors acting as therapeutic molecular chaperones to support the folding, assembly and trafficking of proteins (including ion channels) has been demonstrated for Niemann-Pick C disease and congenital hyperinsulinism (SUR1-KATP channel mutants). Also, this therapeutic mechanism is analogous to small molecule correctors for another chloride channel, CFTR (VX-659/VX-445, Vertex Pharmaceuticals), which is proving to be a breakthrough therapeutic approach for cystic fibrosis.

Through structure activity relationship (SAR) and in silica molecular docking studies, we identified hotspots on opposing ends of the SN-401 molecule that interact with separate regions of the SWELL1-LRRC8 complex: the carboxylate group with R103 in multiple LRRC8 subunits at a constriction in the pore, and the cyclopentyl group within the hydrophobic cleft formed by adjacent LRRC8 monomers; functioning like a molecular staple or tether to bind and stabilize loosely associated SWELL1 homomers (especially in the setting of T2D) into a more rigid hexameric structure. Indeed, the cryo-EM structure obtained in lipid nanodiscs required DCPIB/SN-401 binding in order to obtain images of sufficient spatial resolution (Kern et al., 2019), which supports the concept that SN-401 stabilizes the SWELL1 homomer. Another advantage provided by SAR studies was identification and synthesis of SN-401 congeners that removed (SN071/SN072) or enhanced (SN-403/406/407) SWELL1-binding, as these provided powerful tools to query SWELL1-on target activity directly in vitro and in vivo, and also validated the proof-of-concept for developing novel SN-401 congeners with enhanced efficacy.

SWELL1-LRRC8 complexes are broadly expressed in multiple tissues, and consist of unknown combinations of SWELL1, LRRC8b, LRRC8c, LRRC8d and LRRC8e, indicating that SWELL1 complexes will be enormously heterogenous. However, SWELL1-LRRC8 stabilizers like SN-401 may be designed to target many, if not all, possible channel complexes since all will contain the elements necessary for SN-401 binding: at least one R103 (from the requisite SWELL1 monomer: carboxyl group binding site), and the nature of the hydrophobic cleft (cyclopentyl binding site), which is conserved among all LRRC8 monomers. Indeed, traced glucose clamps did reveal insulin sensitization effects in multiple tissues, including adipose, skeletal muscle, liver and heart. The increased glucose-uptake in heart is particularly interesting, since this may provide salutary effects on cardiac energetics that could favorably impact both systolic (HFrEF) and diastolic (HFpEF) function in diabetic cardiomyopathy, and thereby potentially improve cardiac outcomes in T2D, as observed with SGLT2 inhibitors.

The current study provides an initial proof-of-concept for pharmacological induction of SWELL1 signaling using SWELL1 modulators (SN-40X congeners) to treat metabolic diseases at multiple homeostatic nodes, including adipose, liver, and pancreatic β-cell. Hence, SN-401 may represent a tool compound from which a novel drug class may be derived to treat T2D, NASH, and other metabolic diseases.

### Example 14: Materials and Methods for Examples 15 to 22

Animals. All the mice were housed in temperature, humidity, and light-controlled room and allowed free access to water and food. Both male and female SWELL1fl/fl (WT), Myl1Cre;SWELL1^{fl/fl} (Myl1 KO), Myf5Cre;SWELL1^{fl/fl} (skeletal muscle targeted SWELL1 KO), were generated and used in these studies. Myl1Cre (JAX #24713) and Myf5Cre (JAX #007893) mice were purchased from Jackson labs. For high-fat diet (HFD) studies, we used Research Diets Inc. (Cat #D12492) (60 kcal % fat) regimen starting at 14 weeks of age.

Generation of CRISPR/Cas9-mediated SWELL1 floxed (SWELL1fl/fl) mice. SWELL1fl/fl mice were generated as previously described (Zhang et al., 2017). Briefly, SWELL1 intronic sequences were obtained from Ensembl Transcript ID ENSMUST00000139454. All CRISPR/Cas9 sites were identified using ZiFit Targeter Version 4.2. Pairs of oligonucleotides corresponding to the chosen CRISPR-Cas9 target sites were designed, synthesized, annealed, and cloned into the pX330-U6-Chimeric_BB-CBh-hSpCas9 construct (Addgene plasmid #42230), following the protocol detailed in Cong et al., 2013. CRISPR-Cas9 reagents and ssODNs were injected into the pronuclei of F1 mixed C57/129 mouse strain embryos at an injection solution concentration of 5 ng/μl and 75-100 ng/μl, respectively. Correctly targeted mice were screened by PCR across the predicted loxP insertion sites on either side of Exon 3. These mice were then backcrossed >8 generations into a C57BL/6 background.

Antibodies: Rabbit polyclonal anti-SWELL1 antibody was generated against the epitope QRTKSRIEQGIVDRSE (SEQ ID NO: 13) (Pacific Antibodies). All other primary antibodies were purchased from Cells Signaling: anti-β-actin (#8457s), p-AKT1 (#9018s), Akt1 (#2938s), pAKT2 (#8599s), Akt2 (#3063s), p-AS160 (#4288s), AS160 (#2670s), AMPKα (#5831s), pAMPKα (#2535s), FoxO1(#2880s) and pFoxO1(#9464s), p70 S6 Kinase (#9202s), p-p70 S6 Kinase (#9205s), pS6 Ribosomal (#5364s), GAPDH (#5174s), pErk1/2 (#9101s), Total Erk1/2 (#9102s). Purified mouse anti-Grb2 was purchased from BD (610111s). Purified anti-flag mouse antibody was purchased from sigma. Rabbit IgG Santa Cruz (sc-2027). All primary antibodies were used at 1:1000 dilution, except for anti-flag at 1:2000 dilution. All secondary antibody (anti-rabbit-HRP and anti-mouse-HRP) were used at 1:10000 dilution.

Adenovirus. Adenovirus type 5 with Ad5-CMV-mCherry (1×10¹⁰ PFU/ml), Ad5-CMV-Cre-mCherry (3×10¹⁰ PFU/ml) were obtained from the University of Iowa viral vector core facility. Ad5-CAG-LoxP-stop-LoxP-3×Flag-SWELL1 (1×10¹⁰ PFU/ml) were obtained from Vector Biolabs. Ad5-U6-shGRB2-GFP (1×10⁹ PFU/ml) and Ad5-U6-shSCR-GFP (1×10¹⁰ PFU/ml) were obtained from Vector Biolabs.

Electrophysiology. All recordings were performed in the whole-cell configuration at room temperature, as previously described (Zhang et al., 2017 and Kang et al., 2018). Briefly, currents were measured with either an Axopatch 200B amplifier or a MultiClamp 700B amplifier (Molecular Devices) paired to a Digidata 1550 digitizer, using pClamp 10.4 software. The intracellular solution contained (in mM): 120 L-aspartic acid, 20 CsCl, 1 MgCl₂, 5 EGTA, 10 HEPES, 5 MgATP, 120 CsOH, 0.1 GTP, pH 7.2 with CsOH. The extracellular solution for hypotonic stimulation contained (in mM): 90 NaCl, 2 CsCl, 1 MgCl₂, 1 CaCl₂), 10 HEPES, 5 glucose, 5 mannitol, pH 7.4 with NaOH (210 mOsm/kg). The isotonic extracellular solution contained the same composition as above except for mannitol concentration of 105 (300 mOsm/kg). The osmolarity was checked by a vapor pressure osmometer 5500 (Wescor). Currents were filtered at 10 kHz and sampled at 100 μs interval. The patch pipettes were pulled from borosilicate glass capillary tubes (WPI) using a P-87 micropipette puller (Sutter Instruments). The pipette resistance was ˜4-6 Mil when the patch pipette was filled with intracellular solution. The holding potential was 0 mV. Voltage ramps from ˜100 to +100 mV (at 0.4 mV/ms) were applied every 4 s.

Primary muscle satellite cell isolation: Satellite cell isolation and differentiation were performed as described previously with minor modifications (Hindi et al., 2017). Briefly, gastrocnemius and quadriceps muscles were excised from SWELL1^{flfl} mice (8-10 weeks old) and washed twice with 1×PBS supplemented with 1% penicillin-streptomycin and fungizone (300 μl/100 ml). Muscle tissue was incubated in DMEM-F12 media supplemented with collagenase II (2 mg/ml), 1% penicillin-streptomycin and fungizone (300 μl/100 ml) and incubated at shaker for 90 minutes at 37° C. Tissue was washed with 1×PBS and incubated again with DMEM-F12 media supplemented with collagenase II (1 mg/ml), dispase (0.5 mg/ml), 1% penicillin-streptomycin and fungizone (300 ul/100 ml) in a shaker for 30 minutes at 37° C. Subsequently, the tissue was minced and passed through a cell strainer (70 μm), and after centrifugation; satellite cells were plated on BD Matrigel-coated dishes. Cells were stimulated to differentiate into myoblasts in DMEM-F12, 20% fetal bovine serum (FBS), 40 ng/ml basic fibroblast growth factor (bfgf, R&D Systems, 233-FB/CF), 1× non-essential amino acids, 0.14 mM β-mercaptoethanol, 1× penicillin/streptomycin, and Fungizone. Myoblasts were maintained with 10 ng/ml bfgf and then differentiated in DMEM-F12, 2% FBS, 1× insulin-transferrin-selenium, when 80% confluency was reached.

Cell culture: WT C2C12 and SWELL1 KO C2C12 cell line were cultured at 37° C., 5% CO2 Dulbecco's modified Eagle's medium (DMEM; GIBCO) supplemented with 10% fetal bovine serum (FBS; Atlanta Bio selected) and antibiotics 1% penicillin-streptomycin (Gibco, USA). Cells were grown to 80% confluency and then transferred to differentiation media DMEM supplemented with antibiotics and 2% horse serum (HS; GIBCO) to induce differentiation. The differentiation media was changed every two days. Cells were allowed to differentiate into myotubes for up to 6 days. Subsequently, myotube images were taken for quantification of myotube surface area and fusion index.

Myotube morphology, surface area and fusion index quantification: After differentiation (Day 7), cells were imaged with Olympus IX73 microscope (10× objective, Olympus, Japan). For each experimental condition, 5-6 bright field images were captured randomly from 6 well plate. Myotube surface area was quantified manually with ImageJ software. The morphometric quantification was carried out by an independent observer who was blinded to the experimental conditions. For fusion index, differentiated myotube growing on coverslip were washed with 1×PBS and fixed with 2% PFA. After washing with 1×PBS 3 times, cells were permeabilized with 0.1% TritonX100 for 5 minutes at room temperature and subsequently blocking was done with 5% goat serum for 30 minutes. Cells were stained with DAPI (1 μM) for 15 minutes and after washing with 1×PBS, coverslip were mounted on slides with ProLong Diamond anti-fading agent. Cells were imaged with Olympus IX73 microscope (10× objective, Olympus, Japan) with bright field and DAPI filter. Fusion index (number of nuclei incorporated within the myotube/total number of nuclei present in that view field) were analyzed by ImageJ.

RNA sequencing: RNA quality was assessed by Agilent BioAnalyzer 2100 by the University of Iowa Institute of Human Genetics, Genomics Division. RNA integrity numbers greater than 8 were accepted for RNAseq library preparation. RNA libraries of 150 bp PolyA-enriched RNA were generated, and sequencing was performed on a HiSeq 4000 genome sequencing platform (Illumina). Sequencing results were uploaded and analyzed with BaseSpace (Illumina). Sequences were trimmed to 125 bp using FASTQ Toolkit (Version 2.2.0) and aligned to Mus musculus mmp10 genome using RNA-Seq Alignment (Version 1.1.0). Transcripts were assembled and differential gene expression was determined using Cufflinks Assembly and DE (Version 2.1.0). Ingenuity Pathway Analysis (QIAGEN) was used to analyze significantly regulated genes which were filtered using cutoffs of >1.5 fragments per kilobase per million reads, >1.5 fold changes in gene expression, and a false discovery rate of <0.05. Heatmaps were generated to visualize significantly regulated genes.

Myotube signaling studies: For insulin stimulation, differentiated C2C12 myotubes were incubated in serum free media for 6 h and stimulated with 0 and 10 nM insulin for 15 min; while differentiated primary myotubes were incubated in serum free media for 4 h and stimulated with 0 and 10 nM insulin for 2 h. To examine intracellular signaling upon SWELL1 overexpression (SWELL1 O/E), we overexpressed SWELL1-3×Flag by transducing C2C12 myotubes with Ad5-CAG-LoxP-stop-LoxP-SWELL1-3×Flag (MOI 50-60) and Ad5-CMV-Cre-mCherry (MOI 50-60) and polybrene (4 μg/ml) in DMEM (2% FBS and 1% penicillin-streptomycin) for 36 h. Ad5-CMV-Cre-mCherry alone with polybrene (4 μg/ml) (MOI 50-60) was transduced in WT C2C12 or SWELL1 KO C2C12 as controls. Viral transduction efficiency (60-70%) was confirmed by mCherry fluorescence. Cells were allowed to differentiate further in differentiation media up to 6 days. Myotube images were taken before collecting lysates for further signaling studies. GRB2 knock-down was achieved by transducing myotubes with Ad5-U6-shSCR-GFP (Control, MOI 50-60) or Ad5-U6-shSWELL1-GFP (GRB2 KD, MOI 50-60) in DMEM (2% FBS and 1% penicillin-streptomycin) supplemented with polybrene (4 μg/ml) for 24 hour. Cells were allowed to differentiate further in differentiation media up to 6 days. Differentiated myotube images were taken for myotube surface area quantification before collecting the cells for RNA isolation.

Stretch stimulation: C2C12 myotubes were plated in each well of a 6 well BioFlex culture plate. Cells were allowed to differentiate up to 6 days in differentiation media, and then placed into a Flexcell Jr. Tension System (FX-6000T) and incubated at 37° C. with 5% CO₂. C2C12 myotubes on flexible membrane were subjected to either no tension or to static stretch of 5% for 15 minutes. Cells were lysed and protein isolated for subsequent Western blots.

Western blot: Cells were washed with ice cold 1×PBS and lysed in ice-cold lysis buffer (150 mM NaCl, 20 mM HEPES, 1% NP-40, 5 mM EDTA, pH 7.5) with added proteinase/phosphatase inhibitor (Roche). The cell lysate was further sonicated (20% pulse frequency for 20 sec) and centrifuged at 14000 rpm for 20 min at 4° C. The supernatant was collected and estimated for protein concentration using DC protein assay kit (Bio-Rad). For immunoblotting, an appropriate volume of 4× Laemmli (Bio-rad) sample loading buffer was added to the sample (10-20 μg of protein), then heated at 90° C. for 5 min before loading onto 4-20% gel (Bio-Rad). Proteins were separated using running buffer (Bio-Rad) for 2 h at 110V. Proteins were transferred to PVDF membrane (Bio-Rad) and membrane blocked in 5% (w/v) BSA or 5% (w/v) milk in TBST buffer (0.2 M Tris, 1.37 M NaCl, 0.2% Tween-20, pH 7.4) at room temperature for 1 hour. Blots were incubated with primary antibodies at 4° C. overnight, followed by secondary antibody (Bio-Rad, Goat-anti-mouse #170-5047, Goat-anti-rabbit #170-6515, all used at 1:10000) at room temperature for one hour. Membranes were washed 3 times and imaged by chemiluminescence (Pierce) by using a Chemidoc imaging system (BioRad). The images were further analyzed for band intensities using ImageJ software. β-Actin or GAPDH levels were quantified for equal protein loading.

Immunoprecipitation: C2C12 myotubes were plated on 10 cm dishes in complete media and grown to 80% confluency. For SWELL1-3×Flag overexpression, Ad5-CAG-LoxP-stop-LoxP-3×Flag-SWELL1 (MOI 50-60) and Ad5-CMV-Cre-mCherry (MOI 50-60) along with polybrene (4 ug/ml) were added to cells in DMEM media (2% FBS and 1% penicillin-streptomycin) allowed to grow for 36 hours. Cells were then switched to differentiation media for up to 6 days. After that myotubes were harvested in ice-cold lysis buffer (150 mM NaCL, 20 mM HEPES, 1% NP-40, 5 mM EDTA, pH 7.5) with added protease/phosphatase inhibitor (Roche) and kept on ice with gentle agitation for 15 minutes to allow complete lysis. Lysates were incubated with anti-Flag antibody (Sigma #F3165) or control rabbit IgG (Santa Cruz sc-2027) rotating end over end overnight at 4° C. Protein G sepharose beads (GE) were added for 4 h and then samples were centrifuged at 10,000 g for 3 minutes and washed three times with RIPA buffer and re-suspended in laemmli buffer (Bio-Rad), boiled for 5 minutes, separated by SDS-PAGE gel followed by the western blot protocol.

RNA isolation and quantitative RT-PCR: Differentiated cells were solubilized in TRIzol and the total RNA was isolated using PureLink RNA kit (Life Technologies) and column DNase digestion kit (Life Technologies). The cDNA synthesis, qRT-PCR reaction and quantification were carried out as described previously (Zhang et al., 2017). All experiment was performed in triplicate and GAPDH were used as internal standard to normalize the data. All primers used for qRT-PCR are listed in Table 10, below.

TABLE 10
Primers for qRT-PCR

| Gene | Sequence 5′è3′ | SEQ ID NO: |
|---|---|---|
| PGC1a | AGCCGTGACCA CTGACAACGAG | 1 |
| | GCTGCATGGTTCTGAGTGCTAAG | 2 |
| mIGF | GCGATGGGGAAAA TCAGCAG | 3 |
| | CGCCAGGTAGAAGAGGTGTG | 4 |
| MyoHCI | TCCTGCTGTTTCCTTACTTGCT | 5 |
| | GTGATAGAGAGGTAAGCCCAGG | 6 |
| MyoHC IIa | CTCGTCCTGCTTTAAAAAGCTCC | 7 |
| | TCGATTCGCTCCTTTTCGGAC | 8 |
| MyoHC IIb | GTCCTTCCTCAAACCCTTAAAGT | 9 |
| | CATCTCAGCGTCGGAACTCA | 10 |
| GAPDH | TGCACCACCAACTGCTTAG | 11 |
| | GATGCAGGGATGATGTTC | 12 |

Muscle tissue homogenization: Mice were euthanized and gastrocnemius muscle excised and washed with 1×PBS. Muscles tissue were minced with surgical blade and kept in 8 volume of ice cold homogenization buffer (20 mM Tris, 137 mM NaCl, 2.7 mM KCl, 1 mM MgCl₂, 1% Triton X-100, 10% (w/v) glycerol, 1 mM EDTA, 1 mM dithiothreitol, pH 7.8) supplemented with protease/phosphatase inhibitor (Roche). Tissues were homogenized on ice with a Dounce homogenizer (40-50 passes) and incubated for overnight at 4° C. with continuous rotation. Tissue lysate was further sonicated in 20 sec cycle intervals for 2-3 times and centrifuged at 14000 rpm for 20 min at 4° C. The supernatant was collected for protein concentration estimation using DC protein assay kit (Bio-Rad). Due to the high content of contractile protein in this preparation, Coomassie gel staining was performed to demonstrate equal protein loading, and for quantification normalization of Western blots.

Tissue histology: Mice were anesthetized with isoflurane followed by cervical dislocation. Tibialis anterior (TA) muscle was carefully excised and gently immersed into the tissue-tek O.C.T medium placed on wooden cork. Orientation of the tissue maintained while embedding in the medium. Subsequently, wooden cork with tissue gently immersed into the liquid N2 pre-chilled isopentane bath for 10-14 sec and store at −80° C. Tissue sectioning (10 μm) were done with Leica cryostat and all sections collected on positively charged microscope slide for H&E staining as described earlier (Bonetto et al., 2015). Briefly, TA sectioned slides were stained for 2 minutes in hematoxylin, 1 minute in eosin and then dehydrated with ethanol and xylenes. Subsequently, slides were mounted with coverslip and image were taken with EVOS cell imaging microscope (10×objective). For quantification of fiber cross-sectional area, images were processed using ImageJ software to enhance contrast and smooth/sharpen cell boundaries and clearly demarcate muscle fiber cross sectional area. All measurement was performed with an independent observer who was blinded to the identity of the slides.

Exercise tolerance test and inversion testing: Mouse treadmill exercise protocols were adapted from Dougherty et al., 2016. Briefly, mice were first acclimated with the motorized treadmill (Columbus Instruments Exer3/6 Treadmill (Columbus, Ohio) for 3 days by running 10-15 minutes (with 3 minutes interval) for 3 consecutive days at 7 m/min, with the electric shocking grid (frequency 1 Hz) installed in each lane. During the treadmill testing, mice ran with a gradual increase in speed (5.5 m/minute to 22 m/minute) and inclination (0°-15°) at time intervals of 3 minutes each. The total running distance for each mouse was recorded at the end of the experiment. The predefined criteria for removing the mouse from the treadmill and recording the distance travelled was: continuous shock for 5 sec or receiving 5-6 shocks within a time interval of 15 seconds. These mice were promptly removed from the treadmill and total duration and distance were recorded for further analysis. Mouse inversion test was performed using a wire-grid screen apparatus elevated to 50 cm. Mice were stabilized on the screen inclined at 60°, with the mouse head facing towards the base of the screen. The screen was slowly pivoted to 0° (horizontal), such that the mouse was fully inverted and hanging upside down from the screen. Soft bedding was placed underneath the screen to protect mouse from any injury, were they to fall. The inversion test for each mouse was repeated 2 times with an interval of 45 minutes (resting period). The hang time for each mouse was repeated 3 times with an interval of 5-minute. The maximum hanging time limit for each mouse was set for 3 minutes.

Isolated muscle contractile assessment: Soleus muscle was carefully dissected and transferred to a specialized muscle stimulation system (1500A, Aurora Scientific, Aurora, ON, Canada) where physiology tests were run in a blinded fashion. Muscle was immersed in a Ringer solution (in mM) (NaCl 137, KCl 5, CaCl₂) 2, NaH₂PO₄ 1, NaHCO₃ 24, MgSO₄ 1, glucose 11 and curare 0.015) maintained at 37° C. The distal tendon was secured with silk suture to the arm of a dual mode ergometer (300C-LR, Aurora Scientific, Aurora, ON, Canada) and the proximal tendon secured to a stationary post. Muscles were stimulated with an electrical stimulator (701C, Aurora Scientific, Aurora, ON, Canada) using parallel platinum plate electrodes extending along the muscle. Muscle slack length was set by increasing muscle length until passive force was detectable above the noise of the transducer and fiber length was measured through a micrometer reticule in the eyepiece of a dissecting microscope. Optimal muscle length was then determined by incrementally increasing the length of the muscle by 10% of slack fiber length until the isometric tetanic force plateaued. At this optimum length, force was recorded during a twitch contraction and isometric tetanic contraction (300 ms train of 0.3 ms pulses at 225 Hz). The muscle was then fatigued with a bout of repeated tetanic contractions every 10 seconds until force dropped below 50% of peak. At this point, the muscle was cut from the sutures and weighed. This weight, along with peak fiber length and muscle density (1.056 g/cm³), was used to calculate the physiological cross-sectional area (PCSA) and convert to specific force (tension). The experimental data were analyzed and quantified using Matlab (Mathworks), and presented as peak tetanic tension (Tetanic Tension)—peak of the force recording during the tetanic contraction, normalized to PCSA; Time to fatigue (TTF)—time for the tetanic tension to fall below 50% of the peak value during the fatigue test; Half relaxation time (HRT)—half the time between force peak and return to baseline during the twitch contraction.

XF-24 Seahorse assay: Cellular respiration was quantified in primary myotubes using the XF24 extracellular flux (XF) bioanalyzer (Agilent Technologies/Seahorse Bioscience, North Billerica, Mass., USA). Primary skeletal muscle cells isolated from SWELL1^{flfl} mice were plated on BD Matrigel-coated plate at a density of 20×10³ per well. After 24 hours, cells were incubated in Ad5-CMV-mCherry or Ad5-CMV-Cre-mCherry (MOI 90-100) in DMEM-F12 media (2% FBS and 1% penicillin-streptomycin) for 24 hours. Cells were then switched to differentiation media for another 3 days. For insulin-stimulation, cells were incubated in serum free media for 4 h and stimulated with 0 and 10 nM insulin for 2 h. Subsequently, medium was changed to XF-DMEM, and kept in a non-CO₂ incubator for 60 minutes. The basal oxygen consumption rate (OCR) was measured in XF-DMEM. Subsequently, oxygen consumption was measured after addition of each of the following compounds: oligomycin (1 μg/ml) (ATP-Linked OCR), carbonyl cyanide 4-(trifluoromethoxy) phenylhydrazone (FCCP; 1 μM) (Maximal Capacity OCR) and antimycin A (10 μM; Spare Capacity OCR) For the glycolysis stress test, prior to experimentation, cells were switched to glucose-free XF-DMEM and kept in a non-CO₂ incubator for 60 min. Extracellular acidification rate (ECAR) was determined in XF-DMEM followed by these additional conditions: glucose (10 mM), oligomycin (1 μM), and 2-DG (100 mM). Data for Seahorse experiments (normalized to protein) reflect the results of one Seahorse run/condition with 6 replicates.

Metabolic phenotyping: Mouse body composition (fat and lean mass) was measured by nuclear magnetic resonance (NMR); Echo-MRI 3-in-1 analyzer, EchoMRI, LLC). For glucose tolerance test (GTT), mice were fasted for 6 hours and intraperitoneal injection of glucose (lg/kg body weight for lean mice and 0.75 g/kg of body weight for HFD mice) administered. Glucose level was monitored from tail-tip blood using a glucometer (Bayer Healthcare LLC) at the indicated times. For insulin tolerance test (ITT), mice were fasted for 4 hours and after an intra-peritoneal injection of insulin (HumulinR, 1 U/kg for lean mice and 1.25 U/kg for HFD mice) glucose level was measured by glucometer at the indicated times.

Statistics. Data are represented as mean±s.e.m. Two-tail paired or unpaired Student's t-tests were used for comparison between two groups. For three or more groups, data were analyzed by one-way analysis of variance and Tukey's post hoc test. For GTTs and ITTs, 2-way analysis of variance (Anova) was used. A p-value <0.05 was considered statistically significant. *, ** and *** represents a p-value less than 0.05, 0.01 and 0.001 respectively.

### Example 15: SWELL1 is Expressed and Functional in Skeletal Muscle and is Required for Myotube Formation

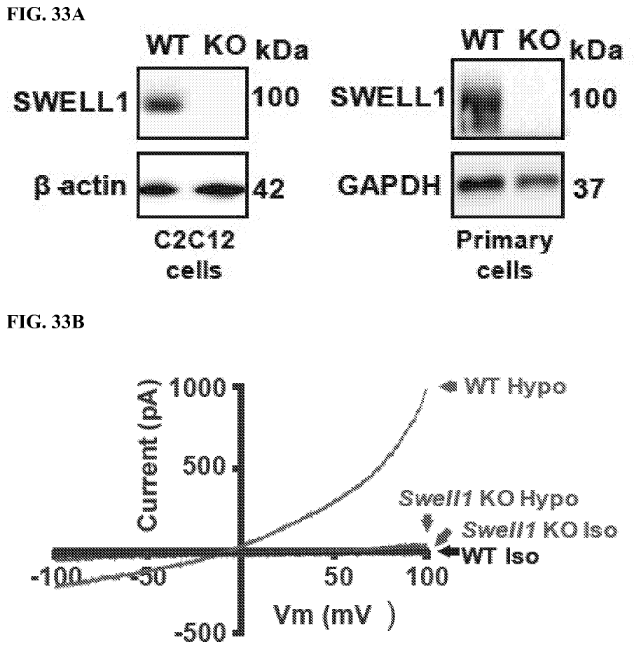

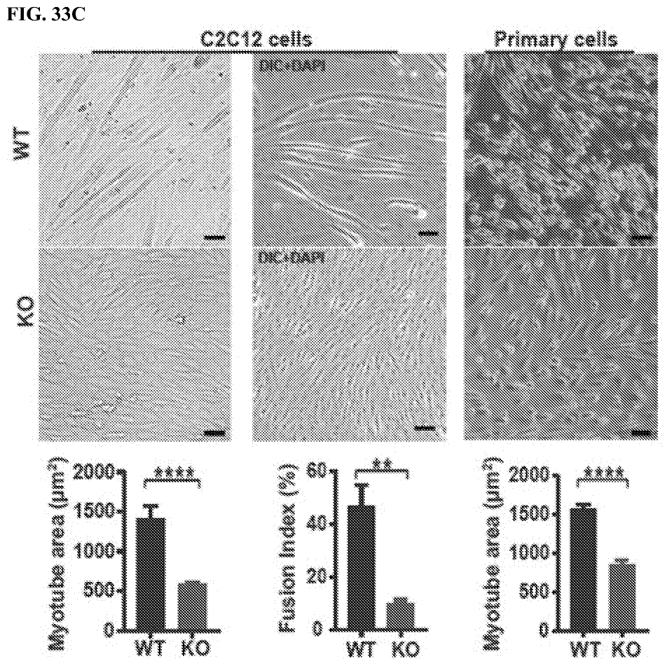

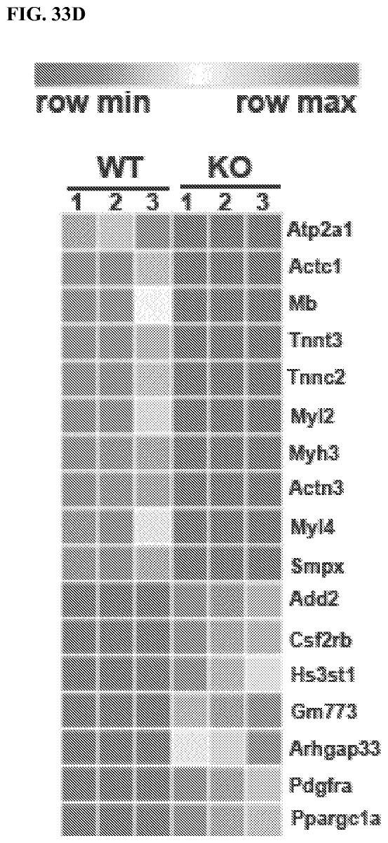

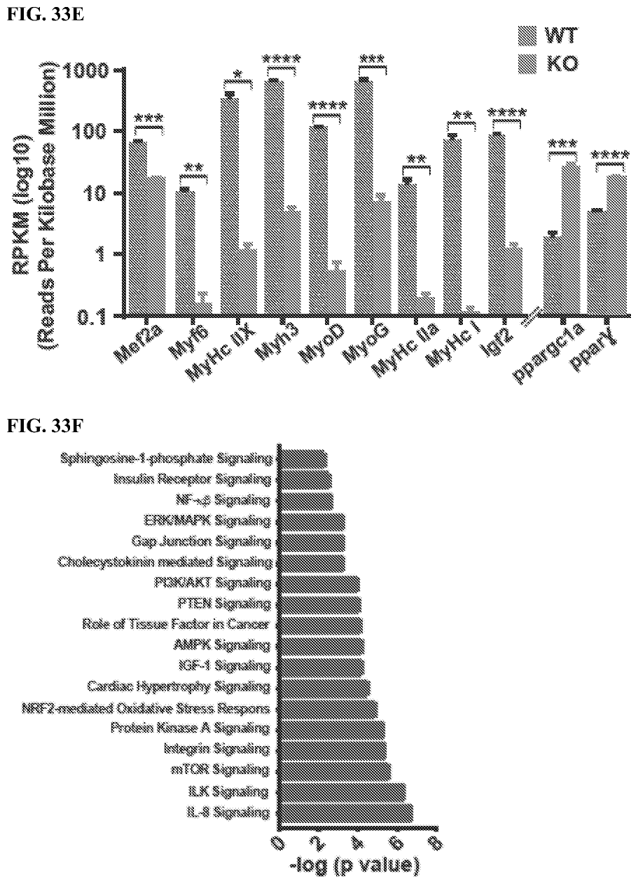

SWELL1 (LRRC8a) is the essential component of a hexameric ion channel signaling complex that encodes I_{Cl,SWELL}, or the volume-regulated anion current (VRAC). While the SWELL1-LRRC8 complex has been shown to regulate cellular volume in response to application of non-physiological hypotonic extracellular solutions, the physiological function(s) of this ubiquitously expressed ion channel signaling complex remain unknown. To determine the function of the SWELL1-LRRC8 channel complex in skeletal muscle, we genetically deleted SWELL1 from C2C12 mouse myoblasts using CRISPR/cas9 mediated gene editing as described previously (Zhang et al., 2017 and Kim et al., 2000), and from primary skeletal muscle cells isolated from SWELL1^{flfl} mice transduced with adenoviral Cre-mCherry (KO) or mCherry alone (WT control) (Zhang et al., 2017). SWELL1 protein Western blots confirmed robust SWELL1 ablation in both SWELL1 KO C2C212 myotubes and SWELL1 KO primary skeletal myotubes (FIG. 33A). Next, whole-cell patch clamp revealed that the hypotonically-activated (210 mOsm) outwardly rectifying current present in WT C2C12 myoblasts is abolished in SWELL1 KO C2C12 myoblasts (FIG. 33B), confirming SWELL1 as also required for I_{Cl,SWELL} or VRAC in skeletal muscle myoblasts. Remarkably, SWELL1 ablation is associated with impaired myotube formation in both C2C12 myoblasts and in primary skeletal satellite cells (FIG. 33C), with an 58% and 45% reduction in myotube area in C2C12 and skeletal muscle myotubes, respectively, compared to WT. As an alternative metric, myoblast fusion is also markedly reduced by 80% in SWELL1 KO C2C12 compared to WT, as assessed by myotube fusion index (number of nuclei inside myotubes/total number of nuclei; FIG. 33C).

### Example 16: Global Transcriptome Analysis Reveals that SWELL1 Ablation Blocks Myogenic Differentiation and Dysregulates Multiple Myogenic Signaling Pathways

In order to further characterize the observed SWELL1 dependent impairment in myotube formation in C2C12 and primary muscle cells we performed genome-wide RNA sequencing (RNA-seq) of SWELL1 KO C2C12 relative to control WT C2C12 myotubes. These transcriptomic data revealed clear differences in the global transcriptional profile between WT and SWELL1 KO C2C12 myotubes (FIG. 33D), with marked suppression of numerous skeletal muscle differentiation genes including Mef2a (0.2-fold), Myl2 (0.008-fold), Myl3 (0.01-fold), Myl4 (0.008-fold), Actc1 (0.005-fold), Tnnc2 (0.005-fold), Igf2 (0.01-fold) (FIG. 33E). Curiously, this suppression of myogenic differentiation is associated with marked induction of ppargc1α (PGC1α; 14-fold) and PPARγ (3.7-fold). PGC1α and PPARγ are positive regulators of skeletal muscle differentiation, showing that the SWELL1-dependent defect in skeletal muscle differentiation lies downstream of PGC1α and PPARγ. To further define putative pathway dysregulation underlying SWELL1 mediated disruptions in myogenesis we next performed pathway analysis on the transcriptome data. We find that numerous signaling pathways essential for myogenic differentiation are disrupted, including insulin (2×10-3), MAP kinase (5×10-4), PI3K-AKT (1×10-4), AMPK (6×10-5), integrin (3×10-6), mTOR (2×10-6), integrin linked kinase (4×10-7) and IL-8 (1×10-7) signaling pathways (FIG. 33F).

### Example 17: SWELL1 Regulates Multiple Insulin Dependent Signaling Pathways in Skeletal Myotubes

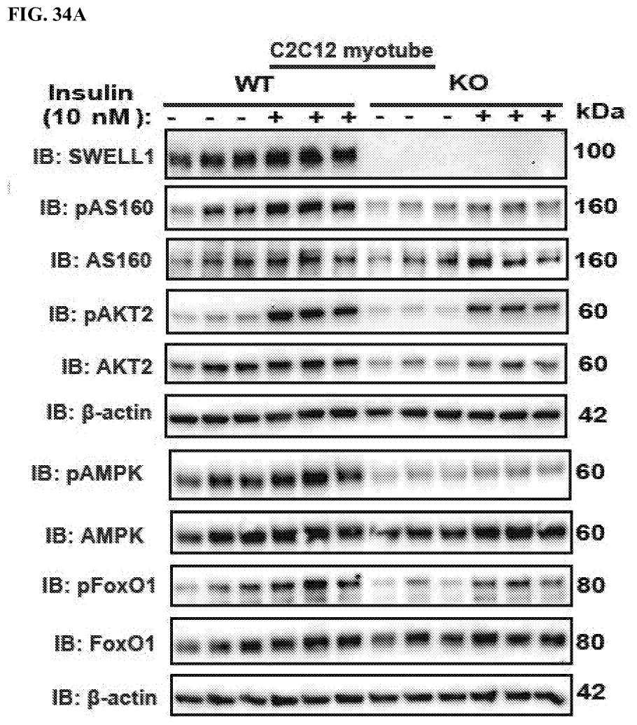

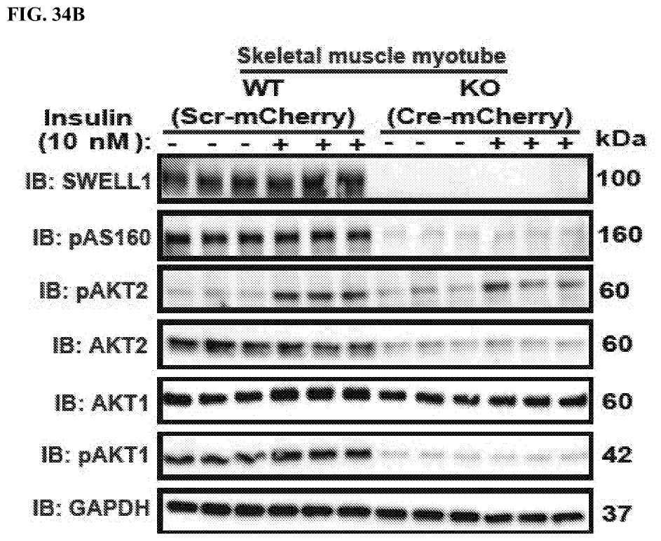

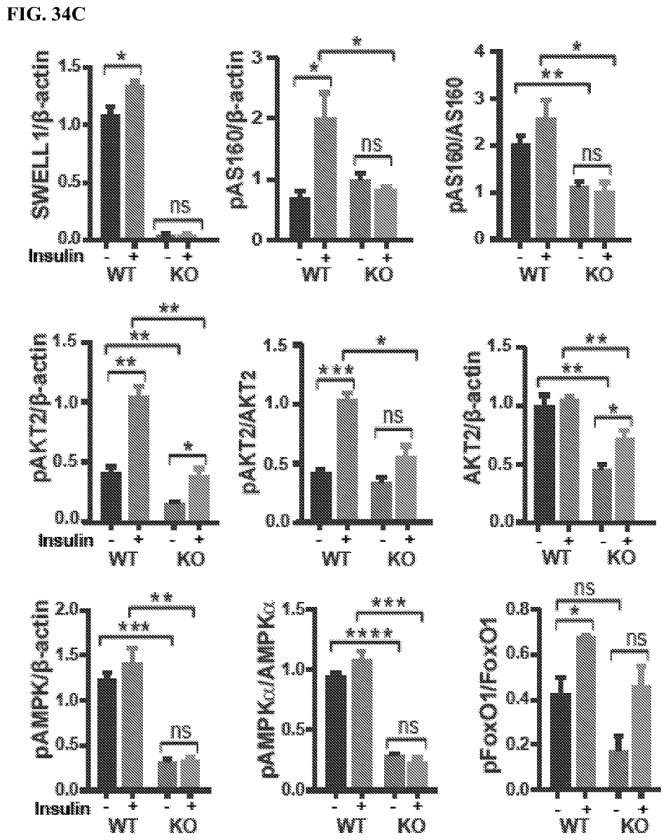

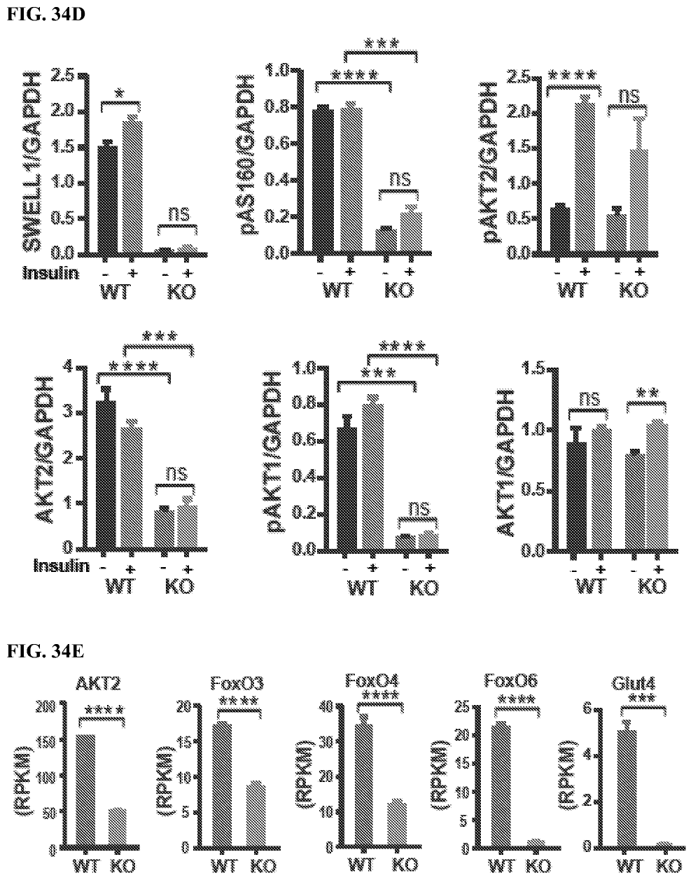

Guided by the results of the pathway analysis, and the fact that skeletal myogenesis and maturation is regulated by insulin-PI3K-AKT-mTOR-MAPK we directly examined a number of insulin-stimulated pathways in WT and SWELL1 KO C2C12 myotubes, including insulin-stimulated AKT2-AS160, FOXO1 and AMPK signaling. Indeed, insulin-stimulated pAKT2, pAS160, pFOXO1 and pAMPK are abrogated in SWELL1 KO myotubes compared to WT C2C12 myotubes (FIGS. 34A and 34C). Importantly, insulin-AKT-AS160 signaling is also diminished in SWELL1 KO primary skeletal muscle myotubes compared to WT primary myotubes (FIGS. 34B&34D), consistent with the observed differentiation block (FIG. 33C). This confirms that SWELL1-dependent insulin-AKT and downstream signaling is not a feature specific to immortalized C2C12 myotubes, but is also conserved in primary skeletal myotubes. It is also notable that reduction in total AKT2 protein is associated with SWELL1 ablation in both C2C12 and primary skeletal muscle cells, and this is consistent with 3-fold reduction in AKT2 mRNA expression observed in RNA sequencing data (FIG. 34E). Moreover, transcription of a number of critical insulin signaling and glucose homeostatic genes are suppressed by SWELL1 ablation, including GLUT4 (SLC2A4, 51-fold), FOXO3 (2-fold), FOXO4 (2.8-fold) and FOXO6 (18-fold) (FIG. 34E). Indeed, FOXO signaling is thought to integrate insulin signaling with glucose metabolism in a number of insulin sensitive tissues. Collectively, these data indicate that impaired SWELL1-dependent insulin-AKT-AS160-FOXO signaling is associated with the observed defect in myogenic differentiation upon SWELL1 ablation in cultured skeletal myotubes, and also predict putative impairments in skeletal muscle glucose metabolism and oxidative metabolism.

### Example 18: SWELL1 Over-Expression in SWELL1 Depleted C2C12 is Sufficient to Rescue Myogenic Differentiation and Augment Intracellular Signaling Above Baseline Levels

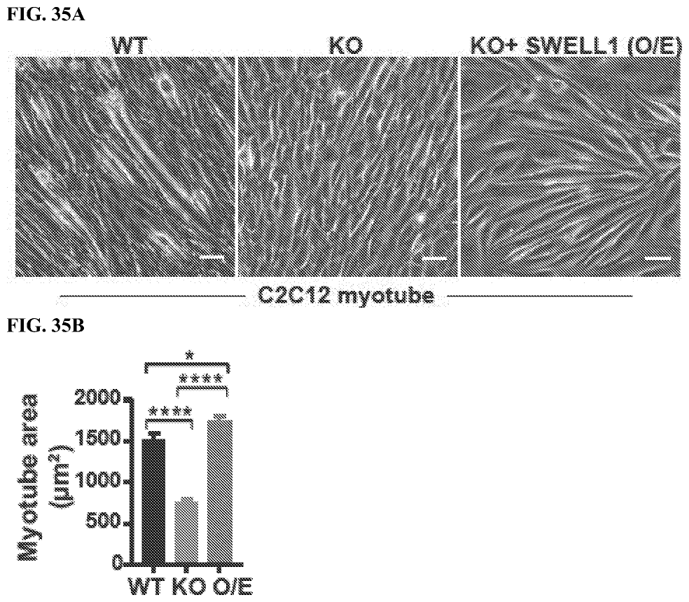

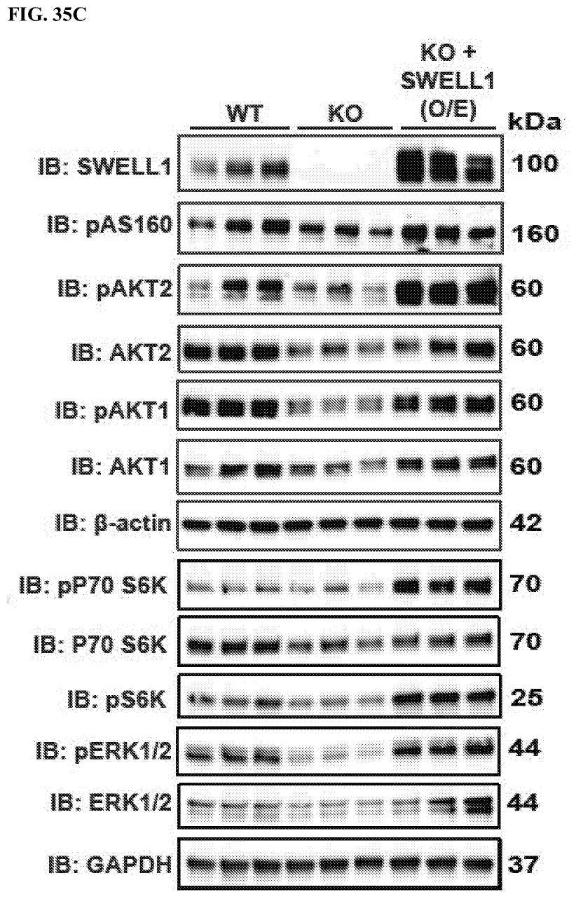

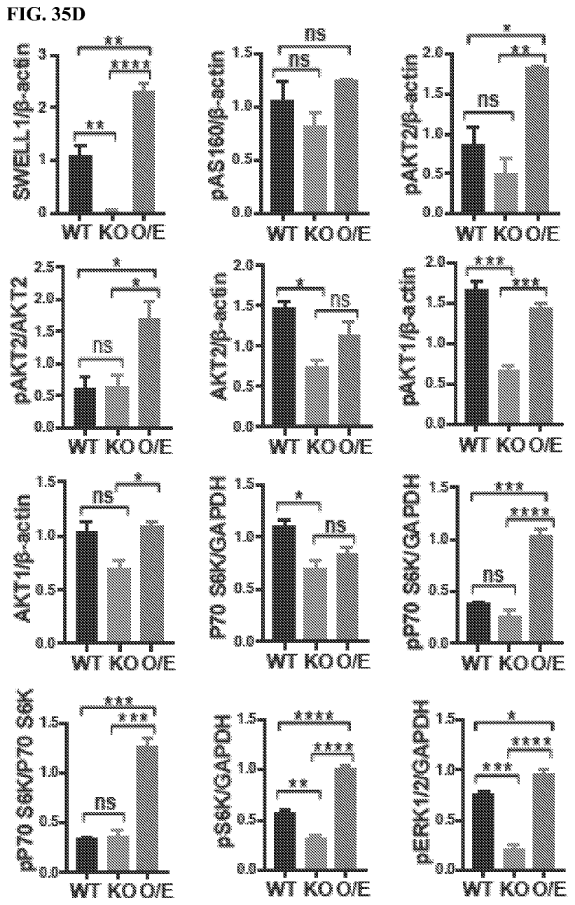

To further validate SWELL1-mediated effects on muscle differentiation and signaling we re-expressed SWELL1 in SWELL1 KO C2C12 myoblasts (SWELL1 O/E) and then examined myotube differentiation and basal activity of multiple intracellular signaling pathways by Western blot, including pAKT1, pAKT2, pAS160, p-p70S6K, pS6K and pERK1/2 as compared to WT and SWELL1 KO C2C12 myotubes. SWELL1 O/E to 2.12-fold WT levels fully rescues myotube development in SWELL1 KO myotubes (FIG. 35A), as quantified by restoration of SWELL1 KO myotube area to levels above WT (FIG. 35B). This rescue of SWELL1 KO myotube development upon SWELL1 O/E (FIGS. 35A and 35B) is associated with either restored (pAS160, AKT2, pAKT1, AKT1, p70S6K) or supra-normal (pAKT2, p-p70S6K, pS6K, pERK1/2) signaling (FIGS. 35C and 35D) compared to WT C2C12 myotubes. These data demonstrate that SWELL1 protein expression level strongly regulates skeletal muscle insulin signaling and myogenic differentiation.

### Example 19: SWELL1-LRRC8 Mediates Stretch-Dependent PI3K-pAKT2-pAS160-MAPK Signaling in C2C12 Myotubes

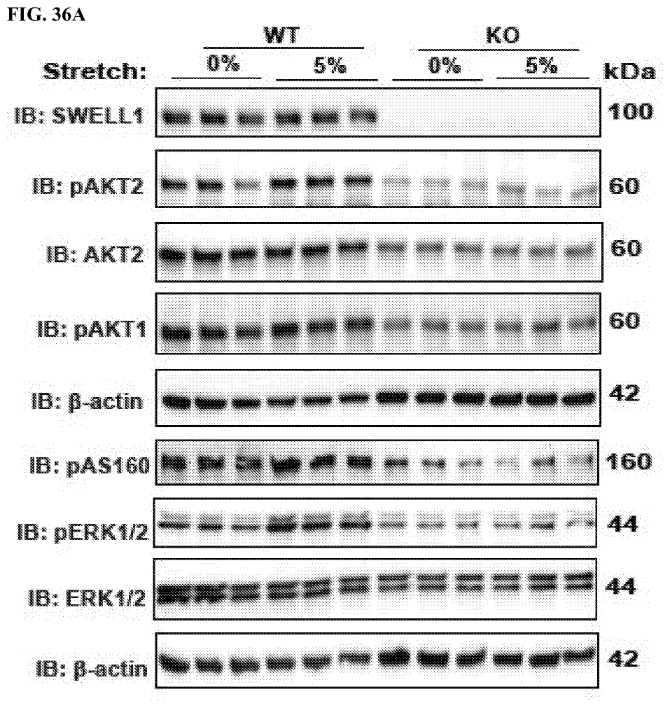

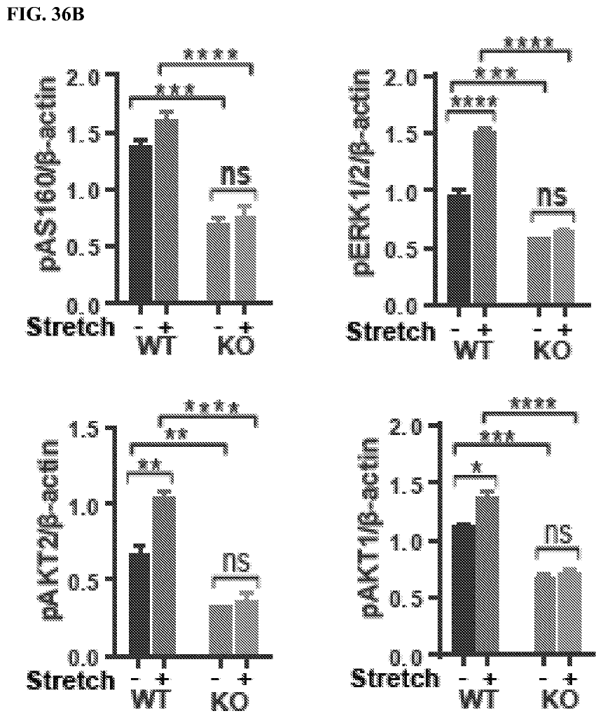

In a cellular context, there are numerous reports that VRAC and the SWELL1-LRRC8 complex that functionally encodes it is mechano-responsive. It is well established that mechanical stretch is an important regulator of skeletal muscle proliferation, differentiation and skeletal muscle hypertrophy and may be mediated by PI3K-AKT-MAPK signaling and integrin signaling pathways. To determine if SWELL1 is also required for stretch-mediated AKT and MAP kinase signaling in skeletal myotubes we subjected WT and SWELL1 KO C2C12 myotubes to 0% or 5% equiaxial stretch using the FlexCell stretch system. Mechanical stretch (5%) is sufficient to stimulate PI3K-AKT2/AKT1-pAS160-MAPK (ERK1/2) signaling in WT C2C12 in a SWELL1-dependent manner (FIGS. 36A and 36B). These data position SWELL1-LRRC8 as a co-regulator of both insulin and stretch-mediated PI3K-AKT-pAS160-MAPK signaling.

### Example 20: SWELL1 Interacts with GRB2 in C2C12 Myotubes and Regulates Myogenic Differentiation

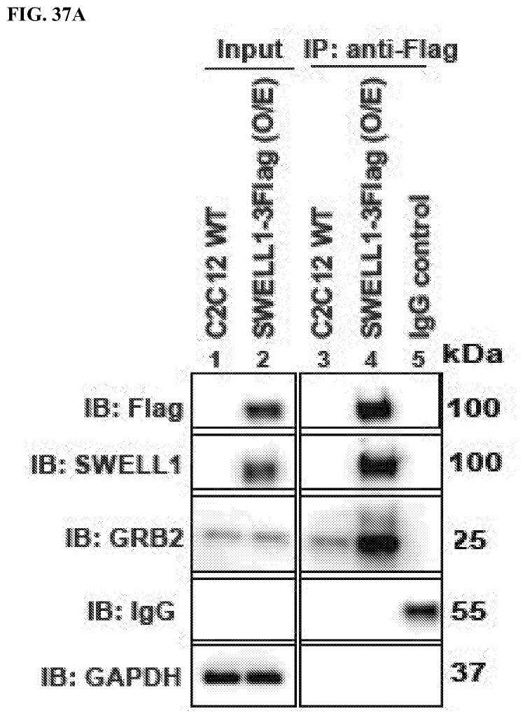

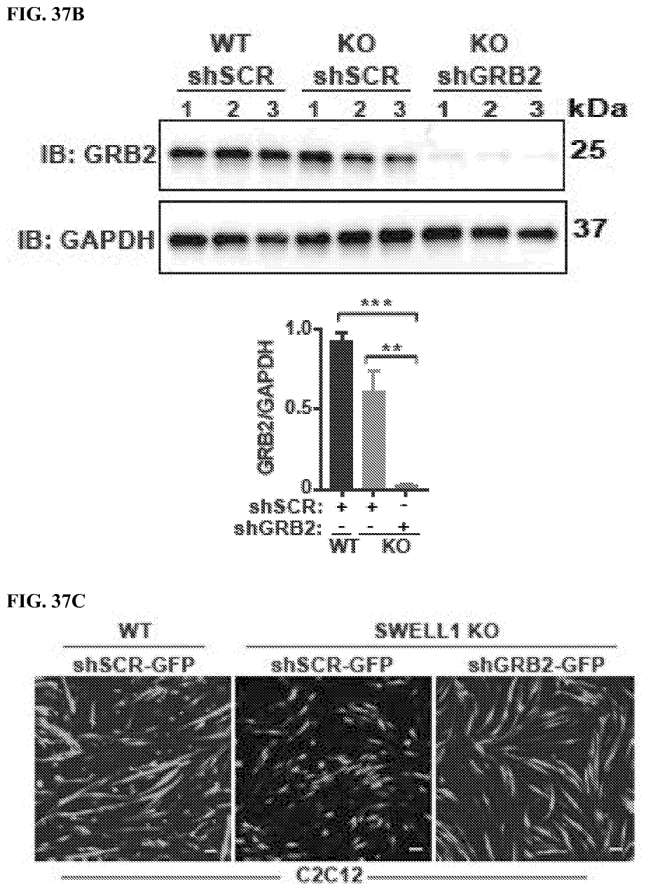

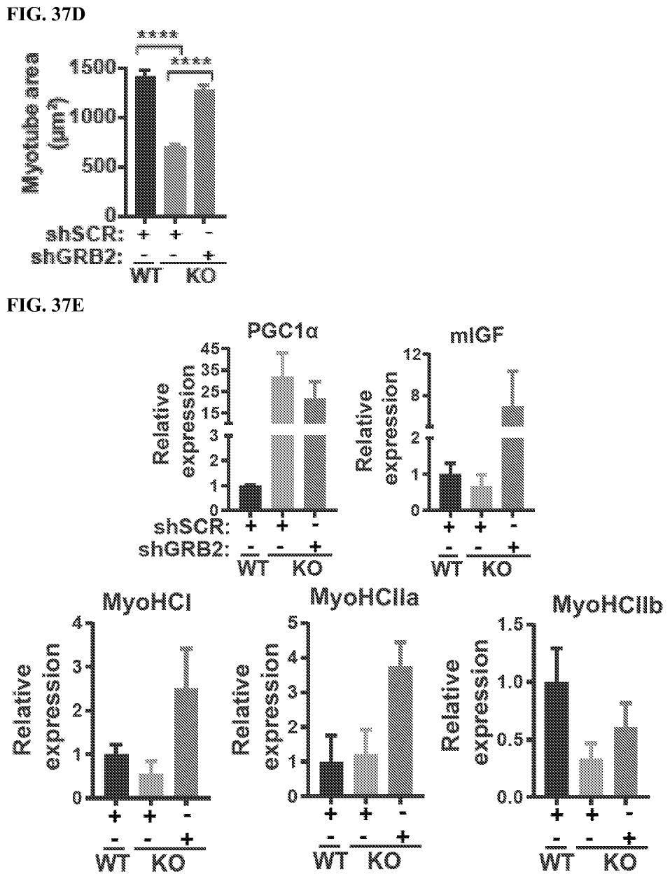

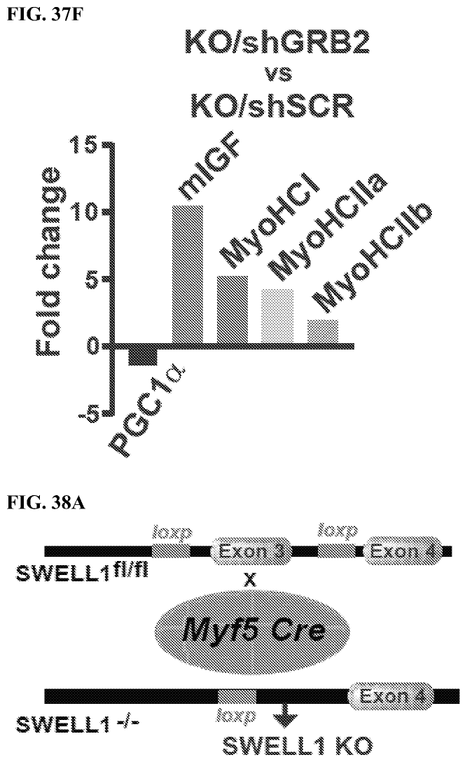

It has been reported earlier in both lymphocyte and adipocytes that the SWELL1-LRRC8 complex interacts with Growth factor Receptor-Bound 2 (GRB2) and regulates PI3K-AKT signaling, whereby GRB2 binds with IRS1/2 and negatively regulates insulin signaling. Indeed, GRB2 knock-down augments insulin-PI3K-MAPK signaling and induces myogenesis and myogenic differentiation genes. To determine if SWELL1 and GRB2 interact in C2C12 myotubes, we overexpressed C-terminal 3×Flag tagged SWELL1 in C2C12 cells followed by immunoprecipitation (IP) with Flag antibody. We observed significant GRB2 enrichment upon Flag IP from lysates of SWELL1-3×Flag expressing C2C12 myotubes, consistent with a GRB2-SWELL1 interaction (FIG. 37A). Based on the notion that SWELL1 titrates GRB2-mediated suppression of AKT/MAPK signaling, and that SWELL1 ablation results in unrestrained GRB2-mediated AKT/MAPK inhibition, we next tested if GRB2 knock-down (KD) may rescue myogenic differentiation in SWELL1 KO C2C12 myotubes. shRNA-mediated GRB2 KD in SWELL1 KO C2C12 myoblasts (SWELL1 KO/shGRB2; FIG. 37B) stimulates myotube formation (FIG. 37C) and increases myotube area (FIG. 37D), to levels equivalent to WT/shSCR (FIGS. 37C and 37D). Similarly, GRB2 KD in SWELL1 KO C2C12 myotubes induces myogenic differentiation markers IGF1, MyoHCl, MyoHClla and MyoHCIIb relative to both SWELL1 KO/shSCR and WT/shSCR (FIGS. 37E and 37F). These data are consistent with GRB2 suppression rescuing myotube differentiation in SWELL1 KO C2C12, and supports a model in which SWELL1 regulates myogenic differentiation by titrating GRB2-mediated signaling.

### Example 21: Skeletal Muscle Targeted SWELL1 Knock-Out Mice have Reduced Skeletal Myocyte Size, Muscle Endurance and Ex Vivo Force Generation

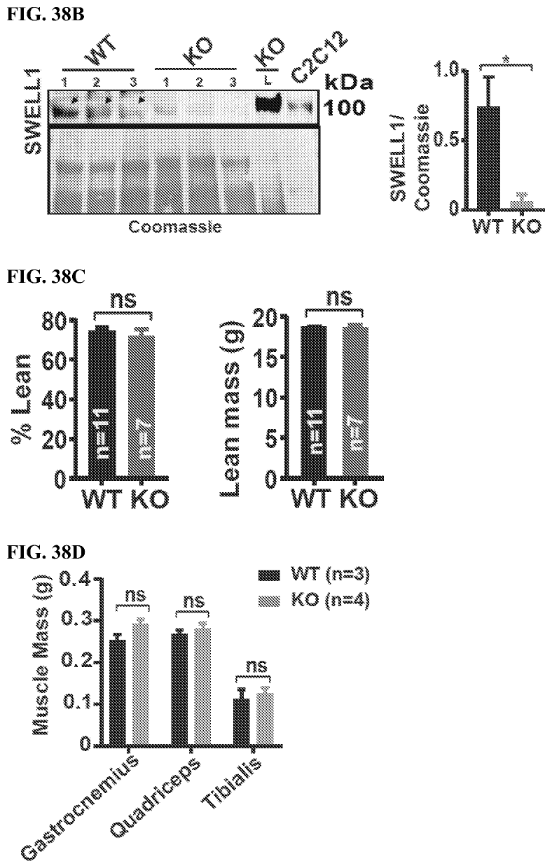

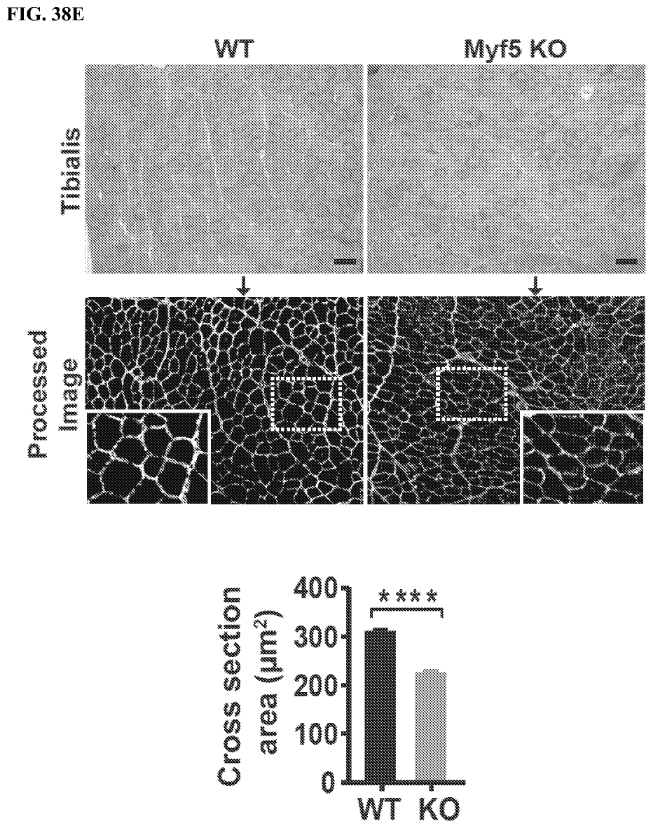

To examine the physiological consequences of SWELL1 ablation in vivo, we generated skeletal muscle specific SWELL1 KO mice using Cre-LoxP technology by crossing Myf5-Cre mice with SWELL1^{fl/fl} mice (Myf5 KO; FIG. 38A), and confirmed robust skeletal muscle SWELL1 depletion in Myf5 KO gastrocnemius muscle, 12.3-fold lower than WT controls (FIG. 38B). Remarkably, in contrast to the severe impairments in skeletal myogenesis observed in both SWELL1 KO C2C12 and primary skeletal myotubes in vitro (FIGS. 33, 35, and 37), Myf5 KO develop skeletal muscle mass comparable to WT littermates, based on Echo/MRI body composition (FIG. 38C) and gross muscle weights (FIG. 38D), and are born at normal mendelian ratios (Table 11, below). However, histological examination reveals a 27% reduction in skeletal myocyte cross-sectional area in Myf5 KO as compared to WT (FIG. 38E), showing a requirement for SWELL1 in skeletal muscle cell size regulation in vivo. This is potentially due to reductions in myotube fusion, as observed in C2C12 and primary skeletal muscle cells in vitro (FIG. 33), but occurring to a lesser degree in vivo. These data indicate that the profound impairments in myogenesis observed in vitro may reflect a very early requirement for SWELL1 signaling in skeletal muscle development (prior to SWELL1 protein elimination by Myf5-Cre mediated SWELL1 recombination), or other fundamental differences in myogenic differentiation processes in vitro versus in vivo.

TABLE 11
Genotypes from Myf5-Cre × SWELL1^{flfl} breeding WT:
SWELL1^{flfl}; KO: Myf5-Cre × SWELL1^{flfl} (Myf5 KO)

| | |
|---|---|
| Male | Female |

| | WT | KO | WT | KO |
|---|---|---|---|---|
| Total: | 18 | 19 | 20 | 15 |
| % | 21.9 | 23.1 | 24.3 | 18.2 |

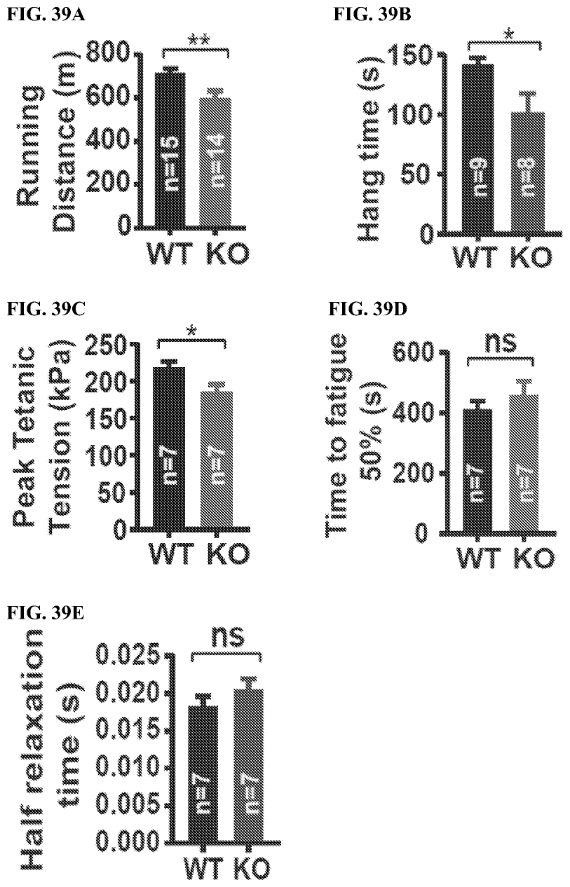

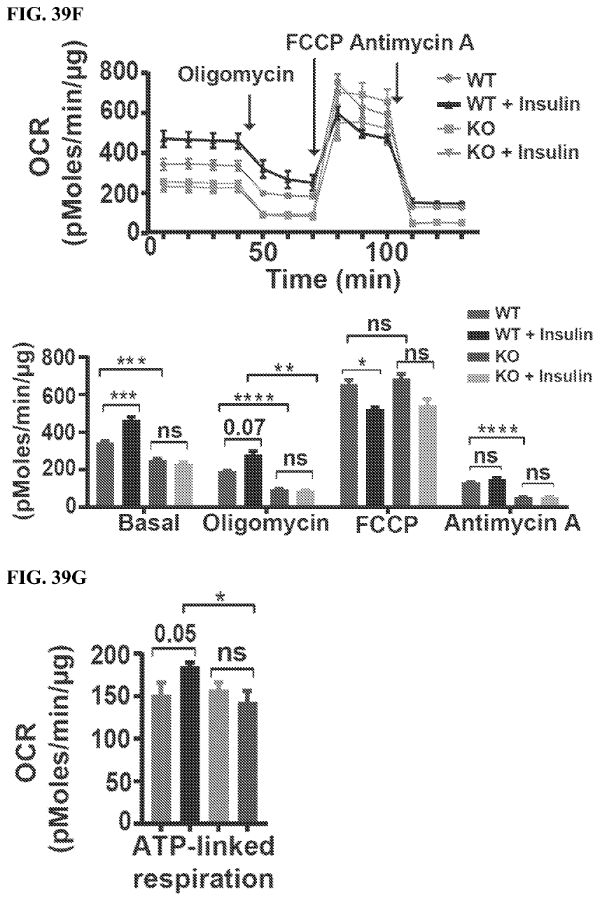

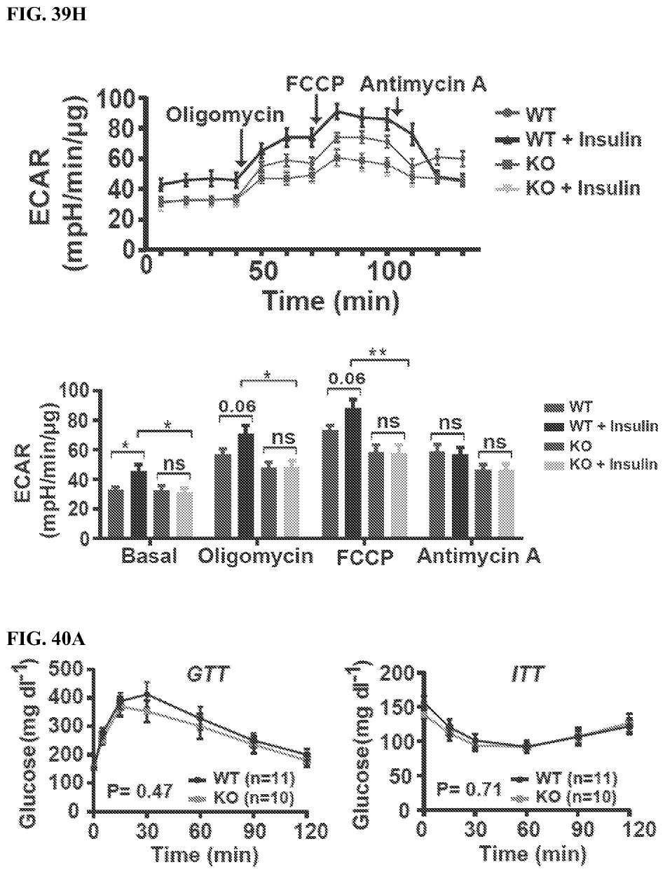

Since insulin signaling is an important regulator of skeletal muscle oxidative capacity and endurance, we next examined exercise tolerance on treadmill testing in SWELL1^{fl/fl} (WT) compared to Myf5 KO mice. Myf5 KO mice exhibit a 14% reduced exercise capacity, compared to age and gender matched WT controls (FIG. 39A). Hang-times on inversion testing are also reduced 29% in Myf5 KO compared to controls, further supporting reduced skeletal muscle endurance upon skeletal muscle SWELL1 depletion in vivo (FIG. 39B). To determine if these reductions in muscle function in vivo are due to muscle-specific functional impairments, we next performed ex vivo experiments in which we isolated the soleus muscle from mice and performed twitch/train testing. We observed that peak developed tetanic tension is 15% reduced in Myf5 KO soleus muscle compared to WT controls (FIG. 39C), showing a skeletal muscle autonomous mechanism, with no difference in time to fatigability (TTF, FIG. 39D) or time to 50% decay (FIG. 39E).

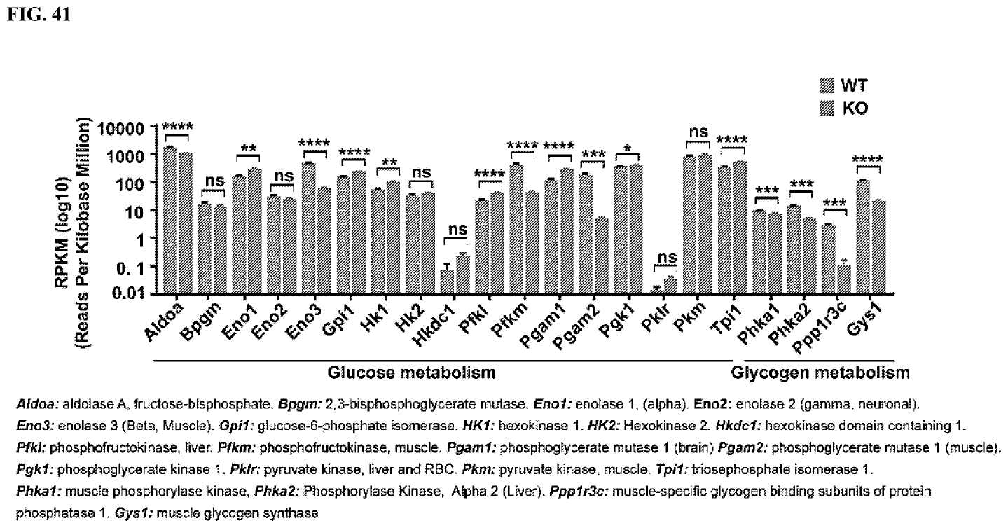

To determine whether these SWELL1 dependent differences in muscle endurance and force were due to impaired oxidative capacity, we next measured oxygen consumption rate (OCR) and extracellular acidification rate (ECAR) in WT and SWELL1 KO primary skeletal muscle cells, under basal and insulin-stimulated conditions (FIG. 39F). Oxygen consumption of SWELL1 KO primary myotubes are 26% lower than WT and, in contrast to WT cells, are unresponsive to insulin-stimulation (FIG. 39F), consistent with abrogation of insulin-AKT/ERK1/2 signaling upon skeletal muscle SWELL1 depletion. These relative changes persist in the presence of Complex V and III inhibitors, Oligomycin and Antimycin A (FIGS. 39F and 39G), showing that insulin-stimulated glycolytic pathways are primarily dysregulated upon SWELL1 depletion. In contrast, FCCP, which maximally uncouples mitochondria, abolishes differences in oxygen consumption between WT and SWELL1 KO primary muscle cells, showing that there might be no differences in functional mitochondrial content in SWELL1 KO muscle. To more directly measure glycolysis, we measured extracellular acidification rate (ECAR) in WT and SWELL1 KO primary myotubes. Insulin-stimulated ECAR increases are abolished in SWELL1 KO compared to WT cells, and these differences persist independent of electron transport chain modulators (FIG. 39H). These data show that SWELL1 regulation of skeletal muscle cellular oxygen consumption occurs at the level of glucose metabolism—potentially via SWELL1-dependent insulin-PI3K-AKT-AS160-GLUT4 signaling, glucose uptake and utilization. These findings in primary skeletal muscle cells are supported by marked transcriptional suppression numerous glycolytic genes: Aldoa, Eno3, GAPDH, Pfkm, and Pgam2; and glucose and glycogen metabolism genes: Phka1, Phka2, Ppp1r3c and Gys1, upon SWELL1 ablation in C2C12 myotubes (FIG. 41).

### Example 22: Skeletal Muscle Targeted SWELL1 Ablation Impairs Systemic Glucose Metabolism and Increases Adiposity

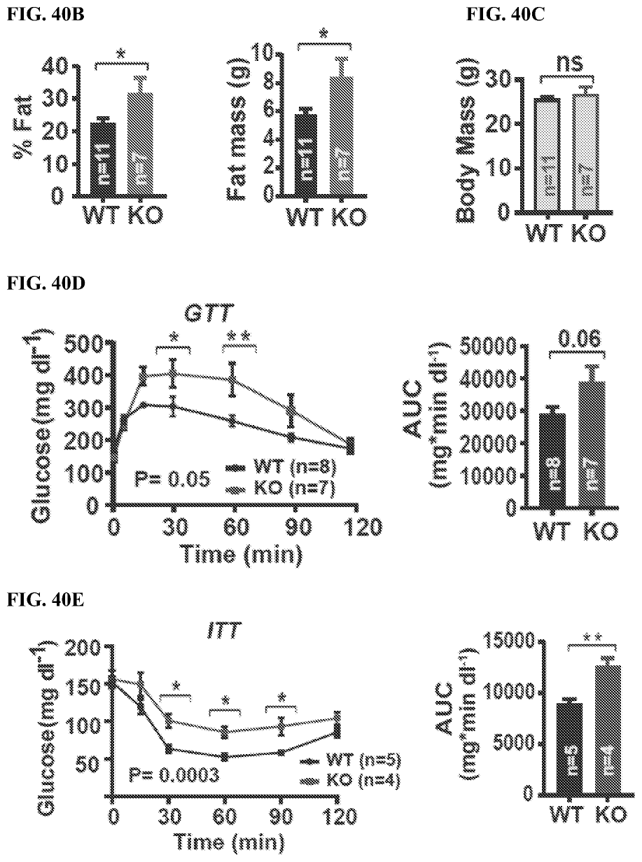

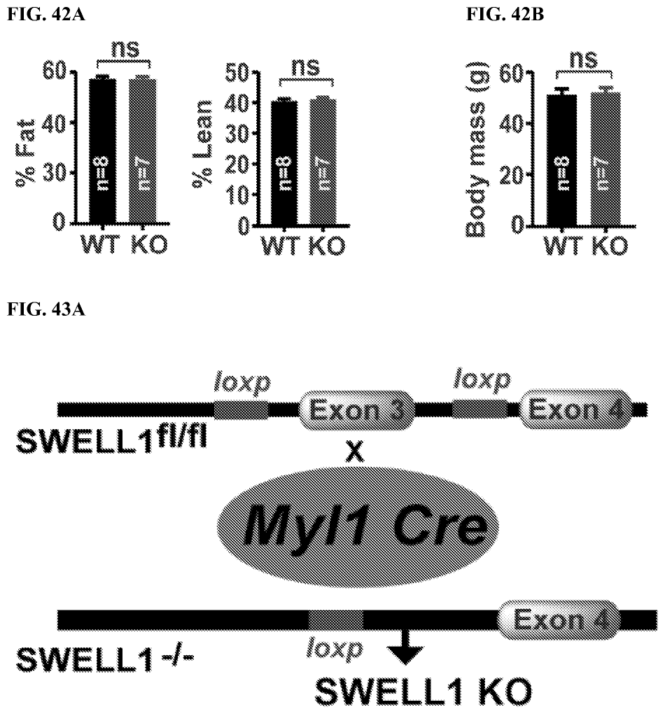

Guided by evidence of impaired insulin-PI3K-AKT-AS160-GLUT4 signaling observed in SWELL1 KO C2C12 and primary myotubes we next examined systemic glucose homeostasis and insulin sensitivity in WT and Myf5 KO mice by measuring glucose and insulin tolerance. On a regular chow diet, there are no differences in either glucose tolerance or insulin tolerance (FIG. 40A) between WT and Myf5 KO mice. However, over the course of 16-24 weeks on chow diet Myf5 KO mice develop 29% increased adiposity based on body composition measurements (FIG. 40B) compared to WT, with no significant difference in lean mass (FIG. 38C) or in total body mass (FIG. 40C). When Myf5 KO mice are raised on a high-fat-diet (HFD) for 16 weeks there is no difference in adiposity observed (FIG. 42) compared to WT mice, but glucose tolerance is impaired (FIG. 40D) and there is mild insulin resistance in HFD Myf5 KO mice as compared to WT (FIG. 40E).

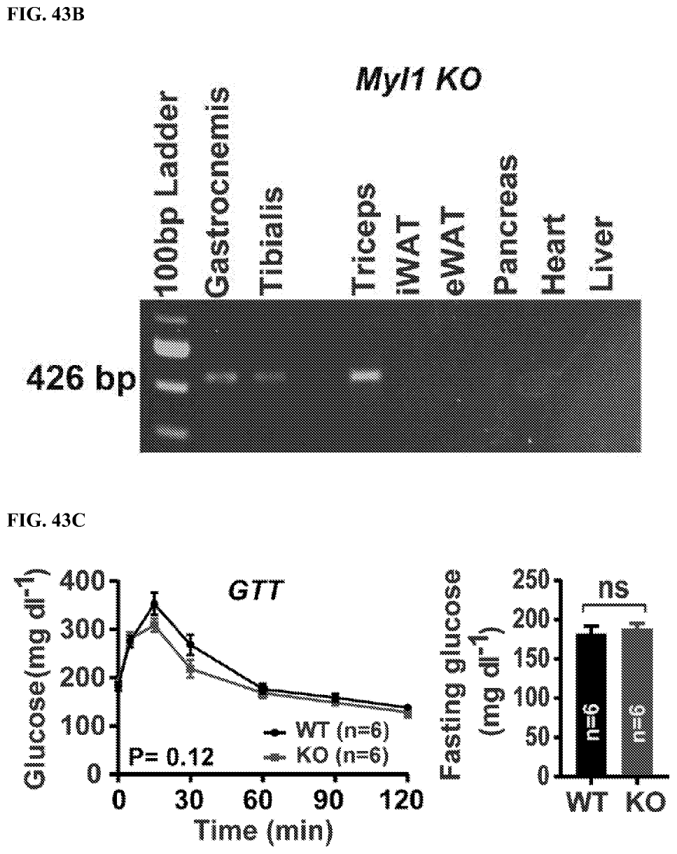

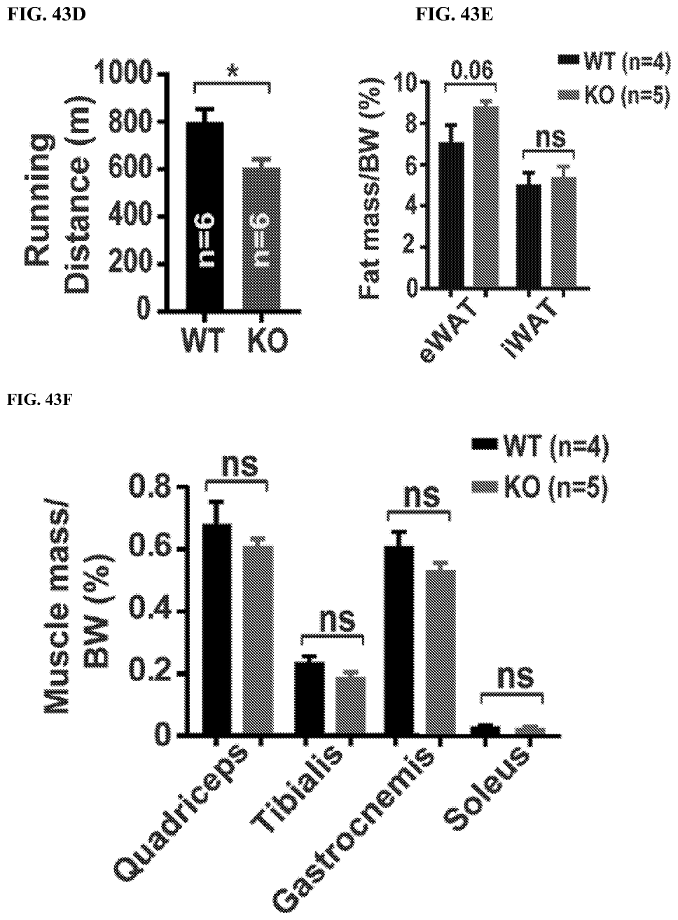

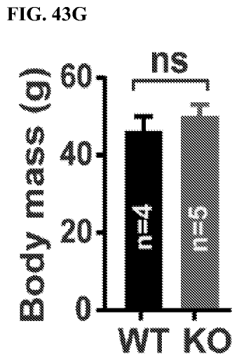

Since Myf5 is also expressed in brown fat, it is possible that these metabolic phenotypes arise from SWELL1-mediated effects in brown fat and consequent changes in systemic metabolism. To rule out this possibility, we repeated a subset of the above experiments in a skeletal muscle targeted KO mouse generated by crossing the Myl1-Cre and SWELL1^{fl/fl} mice (Myl1-Cre;SWELL1^{fl/fl}) or Myl1 KO (FIG. 43A), since Myl1-Cre is restricted to mature skeletal muscle (FIG. 43B), and excludes brown fat. Similar to Myf5 KO mice, Myl1 KO mice fed a regular chow diet, have normal glucose tolerance (FIG. 43C), but exhibit 24% reduced exercise capacity on treadmill testing, as compared to WT (FIG. 43D). Also, Myl1 KO mice develop increased visceral adiposity over time on regular chow, based on 24% increased epididymal adipose mass normalized to body mass (FIG. 43E), with no differences in inguinal adipose tissue, muscle mass (FIG. 43F), or total body mass (FIG. 43G). These data show that impaired skeletal muscle glucose uptake in Myl1 KO and Myf5 KO mice are compensated for by increased adipose glucose uptake and de novo lipogenesis, which contribute to preserved glucose tolerance, at the expense of increased adiposity in skeletal muscle targeted SWELL1 KO mice raised on a regular chow diet. However, overnutrition-induced obesity, and the associated impairments in adipose and hepatic glucose disposal may uncover glucose intolerance and insulin resistance in skeletal muscle targeted SWELL1 KO mice.

### Example 23: Discussion of Examples 15 to 22

Our data reveal that the SWELL1-LRRC8 channel complex regulates insulin/stretch-mediated AKT-AS160-GLUT4, MAP kinase and mTOR signaling in differentiated myoblast cultures, with consequent effects on myogenic differentiation, insulin-stimulated glucose metabolism and oxygen consumption. In vivo, skeletal muscle targeted SWELL1 KO mice have smaller skeletal muscle cells, impaired muscle endurance, and force generation, and are predisposed to adiposity, glucose intolerance and insulin resistance. Insulin/stretch-mediated PI3K-AKT, mTOR signaling are well known to be important regulators of myogenic differentiation, metabolism and muscle function showing impaired SWELL1-AKT-mTOR signaling may underlie the defect in myogenic differentiation. Indeed, consistent with our previous findings and proposed model in adipocytes, in which SWELL1 mediates the interaction of GRB2 with IRS1 to regulate insulin-AKT signaling, SWELL1 also associates with GRB2 in skeletal myotubes, and GRB2 knock-down rescues impaired myogenic differentiation in SWELL1 KO muscle cells. Thus, our working model for SWELL1 mediated regulation of insulin-PI3K-AKT and downstream signaling in adipocytes appears to be conserved in skeletal myotubes. The in vitro phenotype that we observe in CRISPR/cas9 mediated SWELL1 KO C2C12 myotubes and in SWELL1 KO primary myotubes is consistent with the observation of Chen et al., 2019 that used siRNA mediated SWELL1 knock-down to demonstrate that the SWELL1-LRRC8 channel complex is required for myogenic differentiation. However, the ability of both GRB2 KD and SWELL1 O/E to rescue myogenic differentiation and augment insulin-AKT, MAP kinase and mTOR signaling in SWELL1 KO myotubes implicates non-canonical, non-conductive signaling mechanisms. Based on our work and also previous studies, SWELL1 O/E does not increase I_{Cl,SWELL}/VRAC to supranormal levels, although pAKT, pERK1/2 and mTOR levels are augmented by 2-fold to 3-fold above endogenous levels, upon 2-fold SWELL1 O/E in C2C12 myotubes. These data show that alternative/non-canonical signaling mechanisms underlie SWELL1-LRRC8 signaling, as opposed to canonical/conductive signaling mechanisms.

It is also notable that the profound myogenic differentiation block observed upon SWELL1 ablation in both C2C12 myotubes and primary myotubes in vitro is significantly milder in vivo, where only a 30% reduction in skeletal myocyte cross-sectional area is observed, with no change in total muscle mass, or lean content, in Myf5 KO mice. This discordance in phenotype may reflect fundamental differences in the biology of skeletal muscle differentiation in vitro versus the in vivo milieu.

Although overall muscle development is grossly intact in both Myl1 KO and Myf5 KO mice, there is a consistent reduction in exercise capacity, muscle endurance and force generation, and a propensity for increased adiposity over time compared to age and gender matched controls. The observed impairments in exercise capacity in skeletal muscle SWELL1 KO mice are consistent with some level of insulin resistance, as in db/db mice and in humans, and may be due to impaired skeletal muscle glycolysis and oxygen consumption in SWELL1 depleted skeletal muscle. Furthermore, the increased gonadal adiposity, with preserved glucose and insulin tolerance, observed in Myl1 KO and Myf5 KO mice phenocopy both skeletal muscle specific insulin receptor KO mice (MIRKO) and transgenic mice expressing a skeletal muscle dominant-negative insulin receptor mutant, wherein skeletal muscle specific insulin resistance drives re-distribution of glucose from skeletal muscle to adipose tissue, to promote adiposity. In the case of Myf5 KO mice, overnutrition and HFD feeding unmasks this underlying mild insulin resistance and glucose intolerance. Recent findings from skeletal muscle specific AKT1/AKT2 double KO mice indicate that these effects may not attributable to solely to muscle AKT signaling, but potentially involve other insulin sensitive signaling pathways.

In summary, we show that SWELL1-LRRC8 regulates myogenic differentiation and insulin-PI3K-AKT-AS160, ERK1/2, and mTOR signaling in myotubes via GRB2-mediated signaling. In vivo, SWELL1 is required for maintaining normal exercise capacity, muscle endurance, adiposity under basal conditions, and systemic glycemia in the setting of overnutrition. These findings contribute further to our understanding of SWELL1-LRRC8 channel complexes in the regulation of systemic metabolism.

### REFERENCES

Ayala, J. E., Bracy, D. P., Malabanan, C., James, F. D., Ansari, T., Fueger, P. T., McGuinness, O. P., and Wasserman, D. H. (2011). Hyperinsulinemic-euglycemic clamps in conscious, unrestrained mice. Journal of visualized experiments: JoVE.
Bonetto, A., Andersson, D. C. & Waning, D. L. Assessment of muscle mass and strength in mice. Bonekey Rep 4, 732 (2015).
Busch, A. K., Cordery, D., Denyer, G. S., and Biden, T. J. (2002). Expression profiling of palmitate- and oleate-regulat ed genes provides novel insights into the effects of chronic lipid exposure on pancreatic beta-cell function, Diabetes 51, 977-987.
Chen, L., Becker, T. M., Koch, U. & Stauber, T. The LRRC8/VRAC anion channel facilitates myogenic differentiation of murine myoblasts by promoting membrane hyperpolarization. J Biol Chem (2019).
Cong, L., et al. Multiplex genome engineering using CRISPR/Cas systems. Science 339, 819-823 (2013).
Cragoe, E. J., Jr., Gould, N. P., Woltersdorf, O. W., Jr., Ziegler, C., Bourke, R. S., Nelson, L R., Kimelberg, H. K., Waldman, J. B., Popp, A. J., and Sedransk, N. (1982). Agents for the treatment of brain injury. 1. (Aryloxy)alkanoic acids. J Med Chem 25, 567-579.
Dougherty, J. P., Springer, D. A. & Gershengorn, M. C. The Treadmill Fatigue Test: A Simple, High-throughput Assay of Fatigue-like Behavior for the Mouse. Journal of visualized experiments, JoVE (2016).
Egan, W. J., Merz, K. M., Jr., and Baldwin, J. J. (2000). Prediction of drug absorption using multivariate statistics. J Med Chem 43, 3867-3877.
Hindi, L., McMillan, J. D., Afroze, D., Hindi, S. M. & Kumar, A. Isolation, Culturing, and Differentiation of Primary Myoblasts from Skeletal Muscle of Adult Mice. Bio Protoc 7(2017).
Inoue, H., Takahashi, N., Okada, Y., and Konishi, M. (2010). Volume-sensitive outwardly rectifying chloride channel in white adipocytes from normal and diabetic mice. Am J Physiol Cell Physiol 298, C900-909.
Kang, C., et al. SWELL1 is a glucose sensor regulating beta-cell excitability and systemic glycaemia. Nat Commun 9, 367 (2018).
Kern, D. M., Oh, S., Hite, R. K., and Brohawn, S. G. (2019). Cryo-EM structures of the DCPIB-inhibited volume-regulated anion channel LRRC8A in lipid nanodiscs. elife 8.
Kim, J. K., et al. Redistribution of substrates to adipose tissue promotes obesity in mice with selective insulin resistance in muscle. J Clin Invest 105, 1791-1797 (2000).
Kleiner, D. E., Brunt, E. M., Van Natta, M., Behling, C., Contos, M. J., Cummings, O. W., Ferrell, L. D., Liu, Y. C., Torbenson, M. S., Unalp-Arida, A., et al. {2005). Design and validation of a histological scoring system for nonalcoholic fatty liver disease. Hepatology 41, 1313-1321.
Liang, W., Menke, A. L., Driessen, A., Koek, G. H., Lindeman, J. H., Stoop, R., Havekes, L. M Kleemann, R., and van den Hoek, A. M. (2014). Establishment of a general NAFLD scoring system for rodent models and comparison to human liver pat hology. PLoS One 9, e115922.
Lipinski, C. A., Lombardo, F., Dominy, B. W., and Feeney, P. J. {2001). Experimental and computational approaches to estimate solubility and permeability in drug discovery and development settingslPll of original article: S01 69-409X{96)00423-1. The article was originally published in Advanced Drug Delivery Reviews 23 (1997) 3-25.1. Advanced Drug Delivery Reviews 46, 3-26.
Oprea, T. I. {2000). Property distribution of drug-related chemical databases*. Journal of Computer-Aided Molecular Design 14, 251-264.
Rauckhorst, A. J., Gray, L R., Sheldon, R. D., Fu, X., Pewa, A. O., Feddersen, C. R., Dupuy, A. J., Gibson-Corley, K. N., Cox, J. E., Burgess, S. C., et al. (2017). The mitochondrial pyruvate carrier mediates high fat diet-induced increases in hepatic TCA cycle capacity. Mol Metab 6, 1468-1479
Shiota, M. (2012). Measurement of Glucose Homeostasis In Vivo: Combination of Tracers and Clamp Techniques. In Animal Models in Diabetes Research, H.-G. Joost, H. AI-Hasani, and A. Schurmann, eds. (Totowa, N.J.: Humana Press), pp. 229-253.
Veber, D. F., Johnson, S. R., Cheng, H.-Y., Smith, B. R., Ward, K. W., and Kopple, K. D. (2002). Molecular Properties That Influence the Oral Bioavailability of Drug Candidates. Journal of Medicinal Chemistry 45, 2615-2623.
Zhang, Y., et al. SWELL1 is a regulator of adipocyte size, insulin signaling and glucose homeostasis. Nature cell biology 19, 504-517 (2017).

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and methods without departing from the scope of the invention, it is intended that all matter contained in the above description and shown in the accompanying drawings shall be interpreted as illustrative and not in a limiting sense.

What is claimed is:

1. A compound of Formula (I), or salt thereof: 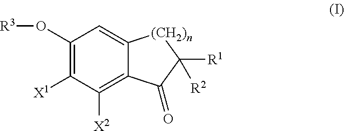
wherein:
R¹ and R² are each independently hydrogen, substituted or unsubstituted alkyl, substituted or unsubstituted alkenyl, substituted or unsubstituted alkoxy, substituted or unsubstituted aryl, substituted or unsubstituted heteroaryl;
R³ is —Y—C(O)R⁴, —Z—N(R⁵)(R⁶), or —Z-A;
R⁴ is hydrogen, substituted or unsubstituted alkyl, —OR⁷, or —N(R⁸)(R⁹);
X¹ and X² are each independently hydrogen, substituted or unsubstituted alkyl, halo, —OR¹⁰, or —N(R¹¹)(R¹²),
R⁵, R⁶, R⁷, R⁸, R⁹, R¹⁰, R¹¹ and R¹² are each independently hydrogen or substituted or unsubstituted alkyl;
Y and Z are each independently a substituted or unsubstituted carbon-containing moiety having at least 2 carbon atoms;
A is a substituted or unsubstituted 5- or 6-membered heterocyclic ring having at least one nitrogen heteroatom, boronic acid or 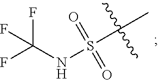 [SMILES: *S(=O)(=O)NC(F)(F)F]
and
n is 1 or 2.

2. The compound of claim 1 wherein at least one of R¹ or R² is a substituted or unsubstituted linear or branched alkyl having at least 2 carbon atoms.

3. The compound of claim 1 or 2 wherein at least one of R¹ or R² is selected from the group consisting of:

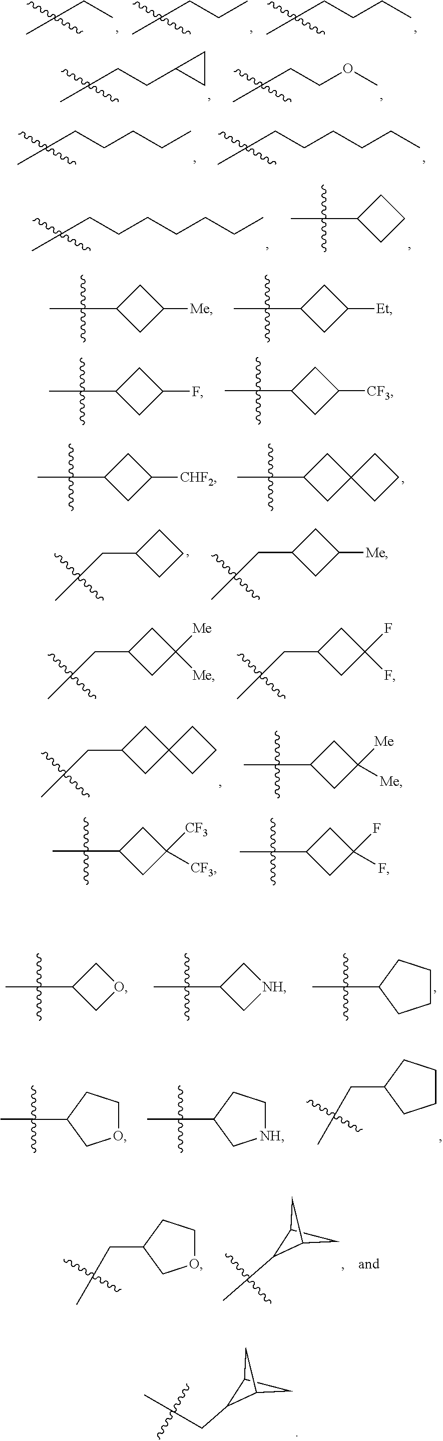

4. The compound of any one of claims 1 to 3 wherein R¹ is hydrogen or a C1 to C6 alkyl.

5. The compound of any one of claims 1 to 4 wherein R¹ is butyl.

6. The compound of any one of claims 1 to 5 wherein R² is cycloalkyl.

7. The compound of any one of claims 1 to 6 wherein R² is cyclopentyl.

8. The compound of any one of claims 1 to 7 wherein R³ is —Y—C(O)R⁴.

9. The compound of any one of claims 1 to 8 wherein R⁴ is —OW or —N(R⁸)(R⁹).

10. The compound of any one of claims 1 to 9 wherein R³ is —Z—N(R⁵)(R⁶).

11. The compound of any one of claims 1 to 10 wherein R³ is —Z-A.

12. The compound of claim 11 wherein A is selected from the group consisting of:

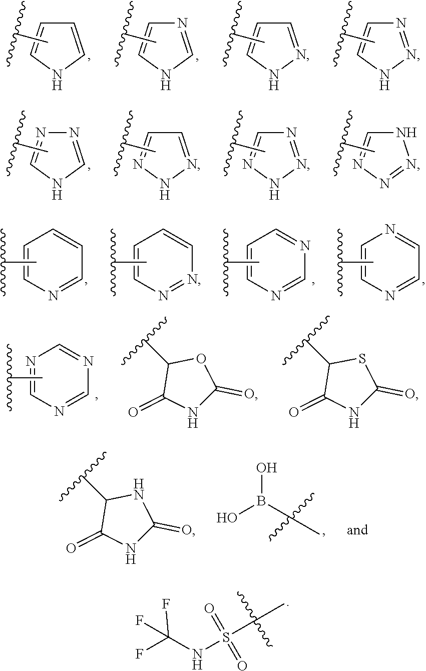

13. The compound of any one of claims 1 to 12, wherein A is selected from the group consisting of

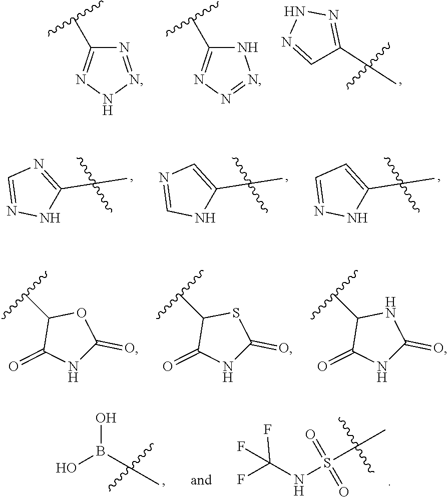

14. The compound of any one of claims 1 to 13 wherein Y and Z are each independently substituted or unsubstituted alkylene having 2 to 10 carbons, substituted or unsubstituted alkenylene having from 2 to 10 carbons, or substituted or unsubstituted arylene.

15. The compound of any one of claims 1 to 14 wherein Y and Z are each independently alkylene having 2 to 10 carbons, alkenylene having from 2 to 10 carbons, or phenylene.

16. The compound of any one of claims 1 to 15 wherein Y and Z are each independently cycloalkylene having 4 to 10 carbons.

17. The compound of any one of claims 1 to 16 wherein Y is an alkylene or an alkenylene having 3 to 8 carbons or 3 to 7 carbons.

18. The compound of any one of claims 1 to 17 wherein Y is an alkylene or any alkenylene having 4 carbons.

19. The compound of any one of claims 1 to 18 wherein Z is an alkylene having 2 to 4 carbons.

20. The compound of any one of claims 1 to 19 wherein Z is an alkylene having 3 or 4 carbons.

21. The compound of any one of claims 1 to 20 wherein Y and Z are each independently selected from the group consisting of

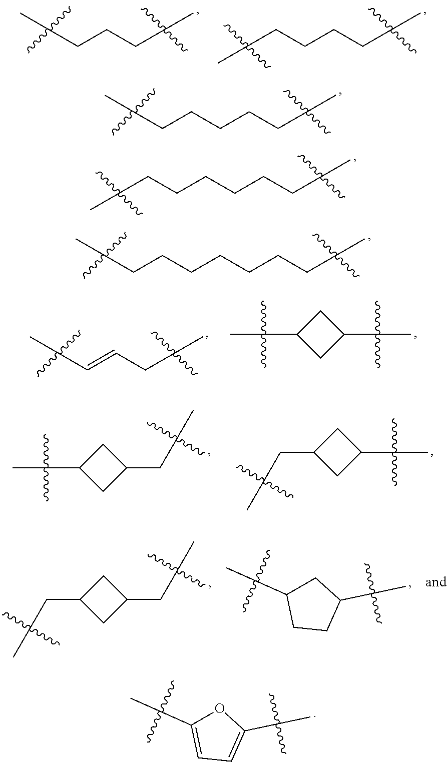

22. The compound of any one of claims 1 to 21 wherein when Y is an alkylene having 2 to 3 carbons then both X¹ and X² are each fluoro or each substituted or unsubstituted alkyl.

23. The compound of any one of claims 1 to 22 wherein R³ is selected from the group consisting of:

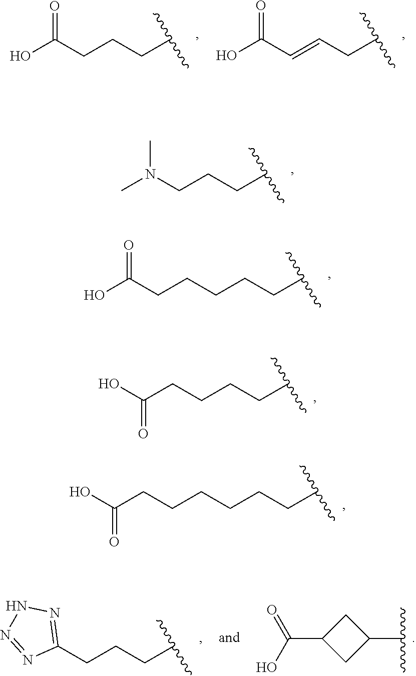

24. The compound of any one of claims 1 to 23 wherein X¹ and X² are each independently substituted or unsubstituted C1 to C6 alkyl or halo.

25. The compound of any one of claims 1 to 24 wherein X¹ and X² are each independently C1 to C6 alkyl, fluoro, chloro, bromo, or iodo.

26. The compound of any one of claims 1 to 25 wherein X¹ and X² are each independently methyl, fluoro, or chloro.

27. The compound of any one of claims 1 to 26 wherein R⁵, R⁶, R⁷, R⁸, R⁹, R¹⁰, R¹¹, and R¹² are each independently hydrogen or alkyl.

28. The compound of any one of claims 1 to 27 wherein R⁵, R⁶, R⁷, R⁸, R⁹, R¹⁰, R¹¹, and R¹² are each independently hydrogen or a C1 to C3 alkyl.

29. The compound of any one of claims 1 to 28 selected from the group consisting of:

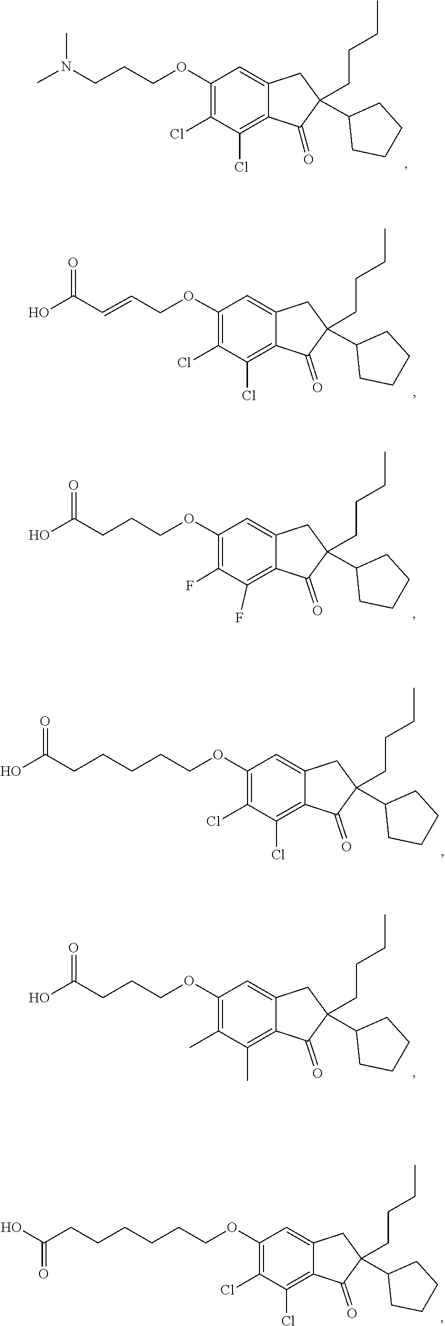 [SMILES: CCCCC1(C2CCCC2)Cc2cc(OCCCN(C)C)c(Cl)c(Cl)c2C1=O ; CCCCC1(C2CCCC2)Cc2cc(OC/C=C/C(=O)O)c(Cl)c(Cl)c2C1=O ; CCCCC1(C2CCCC2)Cc2cc(OCCCC(=O)O)c(F)c(F)c2C1=O ; CCCCC1(C2CCCC2)Cc2cc(OCCCCCC(=O)O)c(Cl)c(Cl)c2C1=O ; CCCCC1(C2CCCC2)Cc2cc(OCCCC(=O)O)c(C)c(C)c2C1=O ; CCCCC1(C2CCCC2)Cc2cc(OCCCCCCC(=O)O)c(Cl)c(Cl)c2C1=O]

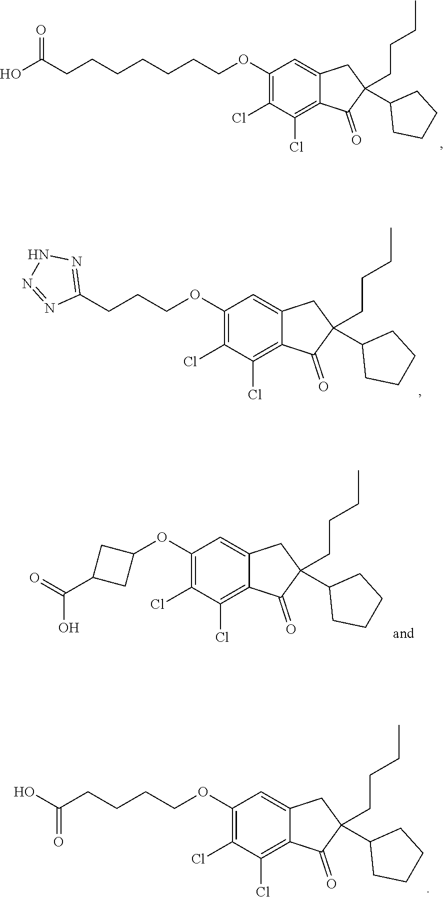 [SMILES: CCCCC1(C2CCCC2)Cc2cc(OCCCCCCCC(=O)O)c(Cl)c(Cl)c2C1=O ; CCCCC1(C2CCCC2)Cc2cc(OCCCc3nn[nH]n3)c(Cl)c(Cl)c2C1=O ; CCCCC1(C2CCCC2)Cc2cc(OC3CC(C(=O)O)C3)c(Cl)c(Cl)c2C1=O ; CCCCC1(C2CCCC2)Cc2cc(OCCCCC(=O)O)c(Cl)c(Cl)c2C1=O]

30. The compound of any one of claims 1 to 29 wherein the compound modulates or inhibits a SWELL1 channel.

31. The compound of claim 30 wherein the compound has a higher potency at modulating or inhibiting a SWELL1 channel than an equivalent amount of DCPIB (4-[2[butyl-6,7-dichloro-2-cyclopentyl-2,3-dihydro-1-oxo-1H-inden-5-yl)oxy]butanoic acid).

32. A method for increasing insulin sensitivity and/or treating obesity, Type I diabetes, Type II diabetes, nonalcoholic fatty liver disease, a metabolic disease, hypertension, stroke, vascular tone, and systemic arterial and/or pulmonary arterial blood pressure and/or blood flow in a subject in need thereof, the method comprising administering to the subject a therapeutically effective amount of the compound of any one of claims 1 to 31.

33. A method for treating an immune deficiency caused by insufficient or inappropriate SWELL1 activity in a subject in need thereof, the method comprising administering to the subject a therapeutically effective amount of the compound of any one of claims 1 to 31.

34. The method of claim 33 wherein the immune deficiency comprises agammaglobulinemia.

35. A method for treating infertility caused by insufficient or inappropriate SWELL1 activity in a subject in need thereof, the method comprising administering to the subject a therapeutically effective amount of the compound of any one of claims 1 to 31.

36. The method of claim 35 wherein the infertility is male infertility caused by abnormal sperm development due to the insufficient or inappropriate SWELL1 activity.

37. A method for treating or restoring exercise capacity and/or improving muscle endurance in a subject in need thereof, the method comprising administering to the subject a therapeutically effective amount of the compound of any one of claims 1 to 31.

38. A method for regulating myogenic differentiation and insulin-P13K-AKT-AS160, ERK1/2 and mTOR signaling in myotubes in a subject in need thereof, the method comprising administering to the subject a therapeutically effective amount of the compound of any one of claims 1 to 31.

39. A method for treating a muscular disorder in a subject in need thereof, the method comprising administering the compound of any one of claims 1 to 31 to the subject.

40. The method of claim 39, wherein the muscular disorder comprises skeletal muscle atrophy.

41. The method of any one of claims 32 to 40 wherein the administration of the compound is sufficient to upregulate the expression of SWELL1 or alter expression of a SWELL1-associated protein.

42. The method of any one of claims 32 to 41 wherein the administration of the compound is sufficient to stabilize SWELL1-LRRC8 channel complexes or a SWELL1-associated protein.

43. The method of any one of claims 32 to 42 wherein the administration of the compound is sufficient to promote membrane trafficking and activity of SWELL1-LRRC8 channel complexes or a SWELL1-associated protein.

44. The method of any one of claims 32 to 43 wherein the SWELL1-associated protein is selected from the group consisting of LRRC8, GRB2, Cav1, IRS1, or IRS2.

45. The method of any one of claims 32 to 44 wherein the administration of the compound is sufficient to augment SWELL1 mediated signaling.


SEQUENCE LISTING (13 sequences)

SEQ ID NO: 1 - DNA, 22 bp, Artificial Sequence
  Synthetic construct
agccgtgaccactgacaacgag

SEQ ID NO: 2 - DNA, 23 bp, Artificial Sequence
  Synthetic construct
gctgcatggttctgagtgctaag

SEQ ID NO: 3 - DNA, 20 bp, Artificial Sequence
  Synthetic construct
gcgatggggaaaatcagcag

SEQ ID NO: 4 - DNA, 20 bp, Artificial Sequence
  Synthetic construct
cgccaggtagaagaggtgtg

SEQ ID NO: 5 - DNA, 22 bp, Artificial Sequence
  Synthetic construct
tcctgctgtttccttacttgct

SEQ ID NO: 6 - DNA, 22 bp, Artificial Sequence
  Synthetic construct
gtgatagagaggtaagcccagg

SEQ ID NO: 7 - DNA, 23 bp, Artificial Sequence
  Synthetic construct
ctcgtcctgctttaaaaagctcc

SEQ ID NO: 8 - DNA, 21 bp, Artificial Sequence
  Synthetic construct
tcgattcgctccttttcggac

SEQ ID NO: 9 - DNA, 23 bp, Artificial Sequence
  Synthetic construct
gtccttcctcaaacccttaaagt

SEQ ID NO: 10 - DNA, 20 bp, Artificial Sequence
  Synthetic construct
catctcagcgtcggaactca

SEQ ID NO: 11 - DNA, 19 bp, Artificial Sequence
  Synthetic construct
tgcaccaccaactgcttag

SEQ ID NO: 12 - DNA, 18 bp, Artificial Sequence
  Synthetic construct
gatgcagggatgatgttc

SEQ ID NO: 13 - protein, 16 aa, Artificial Sequence
  Synthetic construct
QRTKSRIEQGIVDRSE